(19) United States
(12) Patent Application Publication
(10) Pub. No.: US 2014/0162376 A1
(43) Pub. Date: Jun. 12, 2014

(54) COMPOSITIONS AND METHODS TO ASSESS THE CAPACITY OF HDL TO SUPPORT REVERSE CHOLESTEROL TRANSPORT

(71) Applicant: Michael N. Oda, Fairfield, CA (US)

(72) Inventor: Michael N. Oda, Fairfield, CA (US)

(21) Appl. No.: 14/114,494

(22) PCT Filed: Apr. 27, 2012
(86) PCT No.: PCT/US12/35663

§ 371 Date: Feb. 14, 2014

Related U.S. Application Data
(60) Provisional application No. 61/566,581, filed on Dec. 2, 2011.
(60) Provisional application No. 61/481,148, filed on Apr. 29, 2011.

(51) Int. Cl. G01N 33/92

(52) U.S. Cl. CPC G01N 33/92
USPC 436/501; 530/359

(57) ABSTRACT

The invention provides compositions and methods for assessing the capacity of high density lipoprotein (HDL) to support reverse cholesterol transport in blood by measuring exchange if HDL-specific spin-labeled lipoprotein probes and electron paramagnetic spectroscopy. The invention also provides methods to identify individuals at risk for cardiovascular disease, to monitor the treatment of cardiovascular disease and in the development of therapies to treat cardiovascular disease. The invention also provides methods to identify individuals at risk for Alzheimer's disease, to monitor the treatment of Alzheimer's disease and in the development of therapies to treat Alzheimer's disease.

### CROSS-REFERENCE TO RELATED APPLICATIONS

This application claims the priority benefit of U.S. Provisional Patent Application Ser. No. 61/566,581, filed Dec. 2, 2011, and U.S. Provisional Patent Application No. 61/481,148, filed Apr. 29, 2011, each of which is incorporated herein by reference in its entirety.

### STATEMENT REGARDING FEDERALLY SPONSORED RESEARCH OR DEVELOPMENT

This invention was made in part during work supported by grant no. 2 RO1 HL077268-05 from the National Institutes of Health. The government has certain rights in the invention.

### FIELD OF THE INVENTION

The current invention relates to the field of using electron paramagnetic resonance (EPR) spectroscopy to measure the capacity of HDL to support reverse cholesterol transport. EPR spectroscopy can be used to determine the risk of coronary artery disease in an individual.

### BACKGROUND OF THE INVENTION

Studies both in humans [1-5] and genetically modified murine model systems [6-9] have demonstrated the strong association between plasma high-density lipoprotein cholesterol (HDL-C) and coronary artery disease (CAD); the leading cause of mortality in the United States. Population studies have consistently demonstrated that HDL-C level is a more potent independent risk factor for CAD than the level of low-density lipoprotein cholesterol (LDL-C) [1]. While the level of plasma HDL-C is clearly associated with CAD in longitudinal population studies, the level of HDL-C is not, on an individual basis, a good predictor of a patient's predisposition for CAD. Furthermore, both the Framingham Offspring Study and the MESA study found that nearly 40% of CAD patients had normal or elevated HDL-C levels [10, 11]. Similarly, in the IDEAL trial, the highest risk estimates were seen in patients with HDL-C levels above 70 mg/dL [12, 13]. These studies suggest that there may be a dysfunctional pool of high density lipoprotein (HDL) that can lead to abnormally high HDL-C and/or CAD. The realization that current methods for determining HDL-C measures includes healthy and dysfunctional HDL particles, draws into question the validity of HDL-C as a diagnostic and prognostic marker for CAD. Therefore, as presently determined, HDL-C levels alone do not provide all of the information necessary to generate an accurate prognosis for CAD risk at the individual level or treatment for CAD.

The primary means by which healthy HDL is thought to prevent CAD is through reverse cholesterol transport (RCT) (FIG. 1). In the 1970s, Ross and Glomset postulated that cholesterol mobilization via RCT is critical for preventing the onset of atherosclerosis [14]. Shortly thereafter, HDL was identified as the primary mediator of RCT and was proposed as an anti-atherosclerotic lipid complex [15], wherein atherosclerotic protection is conferred by protecting macrophages from LDL-induced apoptosis and preserving endothelial function [16-19]. Through RCT, HDL prevents the generation and accumulation of foam cells that initiate and progress the formation of necrotic core containing atherosclerotic plaques, the principal pathological state underlying CAD.

Although confounded by factors such as age, diabetic status, hypertension, and obesity, indicators of chronic inflammation (C-reactive protein, fibrinogen, white-cell count, and platelet activating factor acetyl hydrolase) significantly associate with increased risk of CAD [20-23]. Chronic inflammation involves the activation of macrophages, which can produce an oxidative environment due in large part to the production of the oxidative enzyme myeloperoxidase (MPO) in the artery intima [24-26]. Generation of an oxidative environment in the intima leads to oxidative modification of HDL [27], wherein apolipoprotein A-I (apoA-I), the main protein component of HDL, is the primary target of oxidation on HDL by MPO [28, 29]. MPO-derived oxidative modification of apoA-I impaired cholesterol mobilization [28, 30-32] via ABCA1, supporting the conclusion that one consequence of chronic inflammation in CAD is to generate dysfunctional HDL that are impaired in their cholesterol efflux capacity.

Interestingly, the laboratories of Drs. Rader and Rothblat recently demonstrated that the ability of human plasma HDL to promote sterol efflux from cultured macrophages varies significantly among individual subjects, despite similar levels of HDL-C and apoA-I [33]. Furthermore, they determined that the sterol efflux capacity of plasma HDL strongly associates with CAD status, independent of HDL-C [34]. This metric of HDL function exhibited a greater inverse correlation odds ratio (0.75; 95% CI) than HDL-C (0.85; 95% CI) and appears to be a more accurate predictor of CAD than HDL-C with a p<0.002 versus p<0.09, respectively. While promising, measuring the HDL sterol efflux capacity of human plasma is a laborious and costly process that is performed using cultured cells. Although, this approach may be informative, it is not necessarily one that can be easily scaled for large sample numbers or efficient throughput.

During HDL's passage through RCT, it undergoes a series of remodeling events resulting from changes in its lipid and protein content (e.g. apoA binding/displacement). Each HDL subclass has unique stability, cholesterol efflux capacity, and enzyme and receptor affinity properties [35-37]. Through these subclass transitions apoA-I undergoes considerable conformational adaptation and this flexibility is essential for ABCA1 mediated cholesterol efflux. Studies have shown that MPO-mediated oxidation of apoA-I impaired this process with a concomitant impact on HDL's ability to mediate cholesterol mobilization via ABCA1 [28, 30]. Recently, a fluorescence-based assay that measures the effects MPO oxidation on HDL rate of apoA-I binding/displacement has been developed [38]. This approach has proven to be informative in assessing the effect of oxidative events on HDL's ability to efflux cholesterol but is of limited use in assessing the efflux capacity of HDL in biological samples. Unfortunately, because of the inherent fluorescence of complex biological fluids including blood plasma, this fluorescence approach cannot be directly applied to clinically relevant samples such as human in vitro blood samples, including, for example, whole blood, serum or plasma.

What is needed is a sensitive assay to measure the reverse cholesterol transport capacity of HDL in in vitro blood samples. Such an assay may be useful in determining the risk of cardiovascular disease including CAD, atherosclerosis, peripheral vascular disease and stroke; monitoring the progression of treatments for cardiovascular disease including CAD, atherosclerosis, peripheral vascular disease and stroke; and in the development of treatments for cardiovascular disease including CAD, atherosclerosis, peripheral vascular disease and stroke.

All references cited herein, including patent applications and publications, are incorporated herein by reference in their entirety.

### BRIEF SUMMARY OF THE INVENTION

The invention provides methods for measuring the capacity of high density lipoprotein (HDL) to support reverse cholesterol transport in a sample (e.g. a biological or synthetic sample as described herein), the method comprising adding a spin-labeled lipoprotein probe to a sample, wherein the spin-labeled lipoprotein probe has high specificity for HDL, and collecting the electron paramagnetic resonance (EPR) spectrum of the sample. The invention provides methods for measuring the capacity of high density lipoprotein (HDL) to support reverse cholesterol transport in blood, the method comprising adding a spin-labeled lipoprotein probe to an in vitro blood sample, wherein the spin-labeled lipoprotein probe has high specificity for HDL, and collecting the electron paramagnetic resonance (EPR) spectrum of the sample. In some embodiments, the method further comprises the step of comparing the binding of the spin-labeled lipoprotein probe to HDL by comparing the spectrum with a positive control and/or a negative control. In some embodiments, the negative control is an EPR spectrum of the spin-labeled lipoprotein probe in a lipid-free or lipid-poor environment. In some embodiments, the positive control is an EPR spectrum of the spin-labeled lipoprotein probe bound to dimyristoylphosphatidyl choline. In some embodiments, the reverse cholesterol transport is a cholesterol efflux potential.

In some embodiments of the invention, an amplitude of a center peak of the EPR spectrum is measured. In some embodiments, a difference in the amplitude of the center peak of the EPR spectrum of the spin-labeled lipoprotein probe in the blood sample compared to the EPR spectrum of a lipid-poor spin-labeled lipoprotein probe is indicative of a difference in the binding of the lipoprotein to the HDL. In other embodiments, an increase in the amplitude of the center peak indicates an increase in the binding of the spin-labeled lipoprotein probe to the HDL. In other embodiments, an increase in the amplitude of the center peak indicates an decrease in the binding of the spin-labeled lipoprotein probe to the HDL. In other embodiments, a decrease in the amplitude of the center peak indicates an increase in the binding of the spin-labeled lipoprotein probe to the HDL. In other embodiments, a decrease in the amplitude of the center peak indicates an decrease in the binding of the spin-labeled lipoprotein probe to the HDL. In some embodiments, the change in amplitude of the center peak is measured in relation to the amplitude of a near peak and/or a far peak that does not change upon binding of the spin-labeled lipoprotein probe to HDL in the blood sample. In some embodiments of the invention, a change in the profile of the EPR spectrum is indicative of a change in the binding of the spin-labeled lipoprotein probe. In some embodiments, a shift of the center peak is indicative of a change in the binding of the spin-labeled lipoprotein probe.

In some embodiments of the invention, the sample is a body fluid; for example blood or cerebral brain fluid (CSF). In some embodiments of the invention, the in vitro blood sample is a whole blood sample. In some embodiments, the in vitro blood sample is a plasma sample. In some embodiments, the in vitro blood sample is a serum sample. In some embodiments, in vitro blood sample has been frozen and thawed one or two times prior to addition of the spin-labeled lipoprotein probe. In some embodiments, the in vitro blood sample is a non-human mammalian blood sample. In some embodiments, the in vitro blood sample is a human blood sample. In some embodiments, the sample is a CSF sample. In some embodiments, the CSF is a non-human mammalian CSF sample. In some embodiments, the CSF is a human mammalian CSF sample.

In some embodiments of the invention, the spin-label is located at a single site on the lipoprotein. In some embodiments, the spin-labeled lipoprotein probe comprises a first spin-label and a second spin label. In some embodiments, the first spin label is located at a first single site on the lipoprotein and the second spin-label is located at a second single site on the lipoprotein. In some embodiments, the spin-label is covalently attached to the lipoprotein. In other embodiments, the spin-label in non-covalently attached to the lipoprotein.

In some embodiments of the invention, the spin-labeled lipoprotein probe comprises an apoA-I or a fragment thereof, wherein the apoA-I or a fragment thereof has high specificity for HDL. In some embodiments, the spin-labeled lipoprotein probe comprises a fragment of apoA-I, wherein the fragment of apoA-I comprises the HDL-binding region of apoA-I. In some embodiments, the spin label is covalently attached to an amino acid at a single site on the apoA-I lipoprotein or fragment thereof. In some embodiments, the spin label is covalently attached to an amino acid residue of the apoA-I lipoprotein from residue 188 to residue 243. In some embodiments, the spin label is covalently attached to an amino acid at position 98, 111 or 217 of the apoA-I lipoprotein. In some embodiments, the spin label is covalently attached to an amino acid at position 26, 44, 64, 98, 101, 111, 167, 217, or 226 of the apoA-I lipoprotein. In some embodiments, the spin label is covalently attached to an amino acid at position 26, 44, 64, 101, 167, or 226 of the apoA-I lipoprotein. In some embodiments, a native amino acid residue at position 98, 111 or 217 has been replaced by a cysteine residue. In some embodiments, a native amino acid residue at position 26, 44, 64, 98, 101, 111, 167, 217, or 226 has been replaced by a cysteine residue. In some embodiments, a native amino acid residue at position 26, 44, 64, 101, 167, or 226 has been replaced by a cysteine residue. In some embodiments, the spin label is covalently attached to a cysteine residue at position 217 of the apoA-I protein. In some embodiments, the spin label is covalently attached to a cysteine residue at position 217 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 217 of the apoA-I protein, and wherein the spin label is (1-oxyl-2,2,5,5-tetramethyl-Δ3-pyrroline-3-methyl)methanethiosulfonate and an increase in amplitude of the center peak of the EPR spectrum indicates an increase in binding of the spin-labeled lipoprotein probe to HDL in the in vitro blood sample. In some embodiments, the spin label is covalently attached to a cysteine residue at position 111 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 111 of the apoA-I protein, and wherein the spin label is (1-oxyl-2,2,5,5-tetramethyl-Δ3-pyrroline-3-methyl) methanethiosulfonate and an increase in amplitude of the center peak of the EPR spectrum indicates an increase in binding of the spin-labeled lipoprotein probe to HDL in the in vitro blood sample. In some embodiments, the spin label is covalently attached to a cysteine residue at position 98 of the apoA-I protein. In some embodiments, the spin label is covalently attached to a cysteine residue at position 98 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 26 of the apoA-I protein. In some embodiments, the spin label is covalently attached to a cysteine residue at position 26 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 44 of the apoA-I protein. In some embodiments, the spin label is covalently attached to a cysteine residue at position 44 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 64 of the apoA-I protein. In some embodiments, the spin label is covalently attached to a cysteine residue at position 64 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 101 of the apoA-I protein. In some embodiments, the spin label is covalently attached to a cysteine residue at position 101 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 167 of the apoA-I protein. In some embodiments, the spin label is covalently attached to a cysteine residue at position 167 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 226 of the apoA-I protein. In some embodiments, the spin label is covalently attached to a cysteine residue at position 226 of the apoA-I protein, and wherein the spin label is (1-oxyl-2,2,5,5-tetramethyl-Δ3-pyrroline-3-methyl)methanethiosulfonate.

In some embodiments, the spin label is attached to a protein by custom amino acid synthesis. In some embodiments, a spin-labeled amino acid residue is incorporated into a polypeptide by using an in vitro expression system or an in vivo expression system.

In some embodiments of the invention, the spin-labeled lipoprotein probe comprises an apoA-II lipoprotein or fragment thereof, wherein the apoA-II or a fragment thereof has high specificity for HDL. In some embodiments, the spin label is covalently attached to an amino acid at a single site on the apoA-II lipoprotein. In some embodiments, the native amino acid residue at the single site in the apoA-II protein has been replaced by a cysteine residue.

In some embodiments of the invention, the spin-labeled lipoprotein probe comprises an apoE lipoprotein or fragment thereof, wherein the apoE or a fragment thereof has high specificity for HDL. In some embodiments, the apoE lipoprotein is an apoE3 lipoprotein. In some embodiments, the spin label is covalently attached to an amino acid at a single site on the apoE lipoprotein. In some embodiments, the native amino acid residue at the single site in the apoE protein has been replaced by a cysteine residue.

In some embodiments of the invention, the spin-labeled lipoprotein probe comprises an apoA-I mimetic, wherein the apoA-I mimetic has high specificity for HDL. In some embodiments, the apoA-I mimetic is selected from the group consisting of 18A, 18A-Pro-18A, 4F, and 4f-Pro-4F. In some embodiments, the spin label is covalently attached to a single site on the apoA-I mimetic.

In some embodiments, the invention provides methods for measuring capacity of high density lipoprotein (HDL) to support reverse cholesterol transport in a sample (e.g. a biological sample or a synthetic sample as described herein), the method comprising adding a spin-labeled lipoprotein probe to an in vitro blood sample, wherein the spin-labeled lipoprotein probe has high specificity for HDL, and collecting the electron paramagnetic resonance (EPR) spectrum of the sample. In some embodiments, the invention provides methods for measuring capacity of high density lipoprotein (HDL) to support reverse cholesterol transport in blood, the method comprising adding a spin-labeled lipoprotein probe to an in vitro blood sample, wherein the spin-labeled lipoprotein probe has high specificity for HDL, and collecting the electron paramagnetic resonance (EPR) spectrum of the sample. In some embodiments, the spin label comprises an atom that bears a free electron. In some embodiments, the atom that bears a free electron is nitrogen. In some embodiments, the spin label is (1-oxyl-2,2,5,5-tetramethyl-Δ3-pyrroline-3-methyl)methanethiosulfonate; (1-oxyl-2,2,5,5-tetramethyl-Δ3-pyrroline-3-methyl) methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; or 3-(2-iodo-acetamido-methyl)-PROXYL, free radical. In some embodiments, the spin-label is a perdeuterated spin-label. In some embodiments, the spin label is attached to an amino acid on the lipoprotein through a thiosulfonate moiety. In some embodiments, the spin label further comprises a spacer moiety between the spin label and the lipoprotein. In some embodiments, the spacer moiety is methane, ethane, propane or butane.

In some embodiments of the invention, the HDL is HDL3.

The invention provides methods for measuring capacity of high density lipoprotein (HDL) to support reverse cholesterol transport in a sample (e.g. a biological sample or a synthetic sample as described herein), the method comprising adding a spin-labeled lipoprotein probe to an in vitro blood sample, wherein the spin-labeled lipoprotein probe has high specificity for HDL, and collecting the electron paramagnetic resonance (EPR) spectrum of the sample. The invention provides methods for measuring capacity of high density lipoprotein (HDL) to support reverse cholesterol transport in blood, the method comprising adding a spin-labeled lipoprotein probe to an in vitro blood sample, wherein the spin-labeled lipoprotein probe has high specificity for HDL, and collecting the electron paramagnetic resonance (EPR) spectrum of the sample. In some embodiments, the spin-labeled lipoprotein probe is added to the in vitro blood sample at a final concentration of about 0.1 mg/ml to about 1.1 mg/ml. In some embodiments, the spin-labeled lipoprotein probe is added to the in vitro blood sample at a final concentration of about 0.3 mg/ml. In some embodiments, the spin-labeled lipoprotein probe is added to the in vitro blood sample at a final concentration greater than about 0.8 mg/ml.

In some embodiments of the invention, the EPR spectrum is collected at one or more timepoints after addition of the spin-labeled lipoprotein probe to the sample. In some embodiments of the invention, the EPR spectrum is collected at one or more timepoints after addition of the spin-labeled lipoprotein probe to the in vitro blood sample. In some embodiments, the EPR spectrum is monitored at one or more of the following times after addition of the spin-labeled lipoprotein probe to the in vitro blood sample: 1.5 minutes, 4 minutes, 6 minutes, 8 minutes, 10 minutes, 30 minutes, or 60 minutes. In some embodiments, a time to reach equilibrium of binding of the spin-labeled lipoprotein probe to the HDL of ten minutes or longer is indicative of HDL with reduced capacity for reverse cholesterol transport. In some embodiments, a time to reach equilibrium of binding of the spin-labeled lipoprotein probe to the HDL of at least two times longer than the time to reach equilibrium of an in vitro blood sample with normal reverse cholesterol transport capacity is indicative of reduced capacity reverse cholesterol transport. In some embodiments, the evaluation is a determination of the degree of binding of spin-labeled lipoprotein probe to the HDL. In some embodiments, the slope of the initial rate of binding is a determination of the affinity of spin-labeled lipoprotein probe to the HDL. In some embodiments, an equilibrium degree of binding of the spin-labeled lipoprotein probe to the HDL of 80% or less compared to binding of the spin-labeled lipoprotein probe in an in vitro blood sample with normal reverse cholesterol transport capacity is indicative of reduced capacity reverse cholesterol transport. In some embodiments, an 80% or less degree of binding of the spin-labeled lipoprotein probe to the HDL at equilibrium compared to binding of the spin-labeled lipoprotein probe in an in vitro blood sample with normal reverse cholesterol transport capacity is indicative of reduced capacity reverse cholesterol transport.

The invention provides methods for measuring capacity of high density lipoprotein (HDL) to support reverse cholesterol transport in a sample (e.g. a biological sample or a synthetic sample as described herein), the method comprising adding a spin-labeled lipoprotein probe to an in vitro blood sample, wherein the spin-labeled lipoprotein probe has high specificity for HDL, and collecting the electron paramagnetic resonance (EPR) spectrum of the sample. The invention provides methods for measuring capacity of high density lipoprotein (HDL) to support reverse cholesterol transport in blood, the method comprising adding a spin-labeled lipoprotein probe to an in vitro blood sample, wherein the spin-labeled lipoprotein probe has high specificity for HDL, and collecting the electron paramagnetic resonance (EPR) spectrum of the sample. In some embodiments, capacity of high density lipoprotein (HDL) to support reverse cholesterol transport in blood is evaluated by a determination of the transition temperature of the HDL, wherein a transition temperature of the HDL of 25° C. or higher is indicative of a reduction in reverse cholesterol transport capacity. In some embodiments, the EPR spectrum is collected at temperatures ranging from 0° C. to 37° C. In some embodiments, the EPR spectrum is collected at 37° C. and then collected at 20° C. and/or 0° C. In some embodiments, the EPR spectrum is collected at 0° C. and then collected at 20° C. and/or 37° C. In some embodiments, the EPR spectrum is collected at 4° C. and then collected at 37° C.

In some embodiments, the in vitro blood sample of the invention further comprises an anti-coagulant. In some embodiments, the anti-coagulant is heparin, coumadin, warfarin, EDTA, citrate or oxalate.

In some aspects, the invention provides methods for determining a risk for developing cardiovascular disease in a first individual; the method comprising a) determining the reverse cholesterol transport capacity of an in vitro blood sample from the first individual according to any above embodiments. In some embodiments, the methods further comprise the step of comparing the reverse cholesterol transport capacity of step a) with the reverse transport capacities of blood samples from one or more second individuals not at apparent risk of cardiovascular disease, wherein a reduction of the reverse cholesterol transport capacity of the in vitro blood sample from the first individual relative to the one or more second individuals is indicative of increased risk of cardiovascular disease. In some embodiments, the first and second individuals are human. In some embodiments, the cardiovascular disease is selected from coronary artery disease, atherosclerosis, peripheral vascular disease, and stroke. In some embodiments, the first individual is diabetic and/or obese. In some embodiments, spectra of the one or more second individuals is historical.

In some aspects, the invention provides methods for monitoring the course of a therapy for cardiovascular disease in an individual undergoing treatment for cardiovascular disease, the method comprising a) determining the reverse cholesterol transport capacity of an in vitro blood sample from the individual according to any one of the above embodiments. In some embodiments, the methods further comprise b) determining the reverse cholesterol transport capacity of an in vitro blood sample from the individual one of more times during and/or after administering the therapy to the individual, wherein an increase in the reverse transport capacity of blood samples from the individual is indicative of therapeutic efficacy. In some embodiments, the individual is human. In some embodiments, the cardiovascular disease is selected from coronary artery disease, atherosclerosis, peripheral vascular disease, and stroke. In some embodiments, the method further comprises determining the reverse cholesterol transport capacity of an in vitro blood sample from the individual before administering the therapy to the individual.

In some aspects, the invention provides, methods for determining efficacy of a known or potential therapy for cardiovascular disease, the method comprising, a) determining the reverse cholesterol transport capacity of an in vitro blood sample from a test individual according to any of the above embodiments, wherein the test animal has been subjected to the therapy. In some embodiments, the test animal has been subjected to the therapy by administering the therapy to the test animal. In some embodiments, the method further comprises b) determining the reverse cholesterol transport capacity of an in vitro blood sample from the test animal one or more times during and/or after administering the therapy to the test animal, wherein an increase in the reverse transport capacity of the in vitro blood sample from the test animal is indicative of therapeutic efficacy.

In some aspects, the invention provides, methods for determining efficacy of a known or potential therapy for cardiovascular disease, the method comprising, a) determining the reverse cholesterol transport capacity of an in vitro blood sample from a test individual according to any of the above embodiments, wherein the therapeutic has been added to the blood sample after removal from the individual and prior to analysis. In some embodiments, the test therapeutic is added to multiple blood samples at different concentrations. In some embodiments, the blood is incubated with the test therapeutic for various amounts of time; for example but not limited to 1 min, 2 min, 3 min, 4 min, 5 min, 6 min, 7 min, 8 min, 9 min, 10 min., or greater than 10 min. In a further embodiment of the embodiments above, an increase in the reverse transport capacity of the in vitro blood sample from the test animal is indicative of therapeutic efficacy.

In some aspects, the invention provides a method determining efficacy of a known or potential therapy for cardiovascular disease, the method comprising, a) determining the reverse cholesterol transport capacity of an in vitro blood sample from a test animal according to the above embodiments, b) administering the therapy to the test animal, c) determining the reverse cholesterol transport capacity of the in vitro blood sample from the test animal one or more times during and/or after administering the therapy to the test animal, wherein an increase in the reverse transport capacity of the in vitro blood sample from the test animal is indicative of therapeutic efficacy. In some embodiments, the test animal is selected from a mouse, a rat, a rabbit, a hamster, a guinea pig, a dog, a cat, and a pig.

In some aspects, the invention provides a kit for measuring an in vitro blood samples capacity of high density lipoprotein (HDL) to support reverse cholesterol transport by EPR, the kit comprising a spin-labeled lipoprotein probe wherein the spin-labeled lipoprotein has high specificity for HDL. In some embodiments, the invention provides a kit for measuring in in vitro blood samples capacity of high density lipoprotein (HDL) to support reverse cholesterol transport by EPR, the kit comprising a spin-label and a lipoprotein, wherein the lipoprotein has high specificity for HDL. In some embodiments, the reverse cholesterol transport is a cholesterol efflux potential.

In some aspects, the invention provides a kit for determining the risk for developing cardiovascular disease in an individual, the kit comprising a spin-labeled lipoprotein probe, wherein the spin-labeled lipoprotein probe is formulated to be added to an in vitro blood sample from the individual and analyzed by EPR. In some embodiments, the individual is a human. In some embodiments, the individual is a non-human mammal.

In some aspects, the invention provides a kit for determining the course of therapy for cardiovascular disease in an individual, the kit comprising a spin-labeled lipoprotein probe, wherein the spin-labeled lipoprotein probe is formulated to be added to an in vitro blood sample from the individual and analyzed by EPR. In some embodiments, the individual is a human. In some embodiments, the individual is a non-human mammal.

In some embodiments of the above aspects, the cardiovascular disease is selected from coronary artery disease, atherosclerosis, peripheral vascular disease, and stroke. In some embodiments, the spin-labeled lipoprotein probe is formulated for use with a whole blood sample.

In some aspects, the invention provides a kit for determining the course of therapy for Alzheimer's disease in an individual, the kit comprising a spin-labeled lipoprotein probe, wherein the spin-labeled lipoprotein probe is formulated to be added to a cerebral spinal fluid from the individual and analyzed by EPR. In some embodiments, the individual is a human. In some embodiments, the individual is a non-human mammal. In some embodiments, the spin-labeled lipoprotein probe is formulated for use with cerebral spinal fluid.

In some embodiments of the above aspects, the spin-labeled lipoprotein probe is formulated for use with a biological sample. In some embodiments of the above aspects, the spin-labeled lipoprotein probe is formulated for use with a plasma sample. In some embodiments, the spin-labeled lipoprotein probe is formulated for use with a serum sample. In some embodiments, the spin-labeled lipoprotein probe is formulated for use with an in vitro blood sample that has been frozen and thawed at least one or two times prior to addition of the spin-labeled lipoprotein probe. In some embodiments, the spin-labeled lipoprotein probe is formulated for use with a mammalian blood sample. In some embodiments, the spin-labeled lipoprotein probe is formulated for use with a human blood sample. In some embodiments of the above aspects, the spin-labeled lipoprotein probe is formulated for use with a CSF sample. In some embodiments of the above aspects, the spin-labeled lipoprotein probe is formulated for use with a synthetic sample. In some aspects, the spin-labeled lipoprotein probe is provided in the kit in a container. In some embodiments, the container is a tube, a flatcell tube or a capillary tube. In some embodiments, the spin-labeled lipoprotein probe is provided in the kit as a dry powder.

In some embodiments of the invention, the kits of the above aspects further comprises instructions for use.

In some embodiments, the kits of the above aspects comprise a spin-labeled lipoprotein probe comprising an apoA-I or a fragment thereof, wherein the apoA-I or fragment thereof has high specificity for HDL. In some embodiments, the spin-labeled lipoprotein probe comprises a fragment of apoA-I, wherein the fragment of apoA-I comprises the HDL-binding region of apoA-I. In some embodiments, the spin label is covalently attached to an amino acid at a single site on the apoA-I lipoprotein. In some embodiments, the spin label is covalently attached to an amino acid at a single site of the apoA-I lipoprotein from residue 188 to residue 243. In some embodiments, the spin label is covalently attached to an amino acid at position 98, 111 or 217 of the apoA-I lipoprotein. In some embodiments, the native amino acid residue at position 98, 111 or 217 has been replaced by a cysteine residue. In some embodiments, the spin label is covalently attached to a cysteine residue at position 217 of the apoA-I protein. In some embodiments, the spin label is covalently attached to a cysteine residue at position 217 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 98 of the apoA-I protein. In some embodiments, the spin label is covalently attached to a cysteine residue at position 98 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 111 of the apoA-I protein, and wherein the spin label is (1-oxyl-2,2,5,5-tetramethyl-Δ3-pyrroline-3-methyl) methanethiosulfonate. In some embodiments, the spin label is covalently attached to an amino acid at position 26, 44, 64, 98, 101, 111, 167, 217, or 226 of the apoA-I lipoprotein. In some embodiments, the native amino acid residue at position 26, 44, 64, 98, 101, 111, 167, 217, or 226 has been replaced by a cysteine residue. In some embodiments, the spin label is covalently attached to an amino acid at position 26, 44, 64, 101, 167, or 226 of the apoA-I lipoprotein. In some embodiments, the native amino acid residue at position 26, 44, 64, 101, 167, or 226 has been replaced by a cysteine residue. In some embodiments, the spin label is covalently attached to a cysteine residue at position 26 of the apoA-I protein. In some embodiments, the spin label is covalently attached to a cysteine residue at position 44 of the apoA-I protein. In some embodiments, the spin label is covalently attached to a cysteine residue at position 64 of the apoA-I protein. In some embodiments, the spin label is covalently attached to a cysteine residue at position 101 of the apoA-I protein. In some embodiments, the spin label is covalently attached to a cysteine residue at position 167 of the apoA-I protein. In some embodiments, the spin label is covalently attached to a cysteine residue at position 226 of the apoA-I protein.

In some embodiments, the kits of the above aspects comprise a spin-labeled lipoprotein probe comprising an apoA-II lipoprotein or fragment thereof, wherein the apoA-II or fragment thereof has high specificity for HDL. In some embodiments, in the spin label covalently attached to an amino acid at a single site on the apoA-II lipoprotein or fragment thereof. In some embodiments, a native amino acid residue at the single site in the apoA-II protein has been replaced by a cysteine residue.

In some embodiments, the kits of the above aspects comprise a spin-labeled lipoprotein probe comprising an apoE lipoprotein or fragment thereof, wherein the apoE or fragment thereof has high specificity for HDL. In some embodiments, the apoE lipoprotein or fragment thereof is an apoE3 lipoprotein or fragment thereof. In some embodiments, the spin label covalently attached to an amino acid at a single site on the apoE lipoprotein. In some embodiments, a native amino acid residue at the single site in the apoE protein has been replaced by a cysteine residue.

In some embodiments, the kits of the above aspects comprise a spin-labeled lipoprotein probe comprising an apoA-I mimetic, wherein the apoA-I mimetic has high specificity for HDL. In some embodiments, the apoA-I mimetic is selected from the group consisting of 18A, 18A-Pro-18A, 4F, and 4f-Pro-4F. In some embodiments, the spin label is covalently attached to a single site on the apoA-I mimetic.

In some embodiments, the kits of the above aspects comprise a spin-labeled lipoprotein comprising an atom that bears a free electron. In some embodiments, the atom that bears a free electron is nitrogen. In some embodiments, the spin label is (1-oxyl-2,2,5,5-tetramethyl-Δ3-pyrroline-3-methyl)methanethiosulfonate; (1-oxyl-2,2,5,5-tetramethyl-Δ3-pyrroline-3-methyl)methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; or 3-(2-iodo-acetamido-methyl)-PROXYL, free radical. In some embodiments, the spin-label is a perdeuterated spin-label. In some embodiments, the spin label is attached to an amino acid on the lipoprotein through a thiosulfonate. In some embodiments, the spin label further comprises a spacer between the spin label and the lipoprotein. In some embodiments, the spacer is methane, ethane, propane or butane.

In some embodiments of the invention, more than 60% of the spin-labeled lipoprotein probe binds HDL. In some embodiments, the HDL is HDL3. In some embodiments, the spin-labeled lipoprotein probe is formulated to be added to the in vitro blood sample at a final concentration of about 0.1 mg/ml to about 1.1 mg/ml. In some embodiments, the spin-labeled lipoprotein probe is formulated to be added to the in vitro blood sample at a final concentration of about 0.3 mg/ml. In some embodiments, the spin-labeled lipoprotein probe is formulated to be added to the in vitro blood sample at a final concentration of greater than about 0.8 mg/ml.

In some embodiments, the kits of the above aspects further comprising an anti-coagulant. In some embodiments, the anti-coagulant is heparin, coumadin, warfarin, EDTA, citrate or oxalate. In some embodiments, the kits further comprising a syringe.

In some aspects, the invention provides a composition comprising an apoA-II lipoprotein or fragment thereof, wherein the apoA-II lipoprotein comprises a spin label, wherein the apoA-II lipoprotein or fragment thereof has high specificity for HDL. In some embodiments, the spin label covalently attached to an amino acid at a single site on the apoA-II lipoprotein or fragment thereof. In some embodiments, a native amino acid residue at the single site in the apoA-II protein has been replaced by a cysteine residue.

In some aspects, the invention provides, a composition comprising an apoA-I mimetic with high specificity for HDL, wherein the apoA-I mimetic comprises a spin label. In some embodiments, the apoA-I mimetic is selected from the group consisting of 18A, 18A-Pro-18A, 4F, and 4f-Pro-4F. In some embodiments, the spin label is covalently attached to a single site on the apoA-I mimetic.

In some aspects, the invention provides a composition comprising an apoA-I lipoprotein or fragment thereof; wherein the apoA-I lipoprotein or fragment thereof comprises a spin-label and wherein the spin-label comprises (1-oxyl-2,2,5,5-tetramethylpyrrolidin-3-yl) methyl methanesulfonate; (−)-(1-oxyl-2,2,5,5-tetramethylpyrrolidin-3-yl)methyl methanesulfonate; or (+)-(1-oxyl-2,2,5,5-tetramethylpyrrolidin-3-yl)methyl methanesulfonate. In some embodiments, the spin-label is covalently attached to an amino acid at a single site of the apoA-I lipoprotein from residue 188 to residue 243. In some embodiments, the spin label is covalently attached to an amino acid at position 98, 111 or 217 of the apoA-I lipoprotein. In some embodiments, the native amino acid residue at position 98, 111 or 217 has been replaced by a cysteine residue. In some embodiments, the spin label is covalently attached to a cysteine residue at position 217 of the apoA-I protein. In some embodiments, the spin label is covalently attached to a cysteine residue at position 98 of the apoA-I protein. In some embodiments, the spin label is covalently attached to a cysteine residue at position 98 of the apoA-I protein, and wherein the spin label is (1-oxyl-2,2,5,5-tetramethyl-Δ3-pyrroline-3-methyl) methanethiosulfonate. In some embodiments, the spin label is covalently attached to an amino acid at position 26, 44, 64, 98, 101, 111, 167, 217, or 226 of the apoA-I lipoprotein. In some embodiments, the native amino acid residue at position 26, 44, 64, 98, 101, 111, 167, 217, or 226 has been replaced by a cysteine residue. In some embodiments, the spin label is covalently attached to an amino acid at position 26, 44, 64, 101, 167, or 226 of the apoA-I lipoprotein. In some embodiments, the native amino acid residue at position 26, 44, 64, 101, 167, or 226 has been replaced by a cysteine residue.

In some aspects, the invention provides, a composition comprising an in vitro blood sample and a spin-labeled lipoprotein probe wherein the spin-labeled lipoprotein has high specificity for HDL. In some embodiments, the in vitro blood sample is a whole blood sample. In some embodiments, the in vitro blood sample is a plasma sample. In some embodiments, the in vitro blood sample is a serum sample. In some embodiments, the in vitro blood sample has been frozen and thawed one or two times. In some embodiments, the in vitro blood sample is a non-human mammalian blood sample. In some embodiments, the mammalian blood sample is a human blood sample.

In some aspects, the invention provides, a composition comprising an in vitro cerebral spinal fluid sample and a spin-labeled lipoprotein probe wherein the spin-labeled lipoprotein has high specificity for HDL. In some embodiments, the in vitro blood sample has been frozen and thawed one or two times. In some embodiments, the in vitro cerebral spinal fluid sample is a non-human mammalian cerebral spinal fluid sample (e.g., rat, mouse, non-human primate). In some embodiments, the mammalian cerebral spinal fluid sample is a human cerebral spinal fluid sample.

In some embodiments of the invention, the spin-labeled lipoprotein probe comprises an apoA-I or a fragment thereof, wherein the apoA-I or fragment thereof has high specificity for HDL. In some aspects, the spin-labeled lipoprotein probe comprises a fragment of apoA-I, wherein the fragment of apoA-I comprised the HDL-binding region of apoA-I. In some embodiments, the spin label is covalently attached to an amino acid at a single site on the apoA-I lipoprotein. In some embodiments, the spin label is covalently attached to an amino acid at a single site of the apoA-I lipoprotein from residue 188 to residue 243. In some embodiments, the spin label is covalently attached to an amino acid at position 98, 111 or 217 of the apoA-I lipoprotein. In some embodiments, the native amino acid residue at position 98, 111 or 217 has been replaced by a cysteine residue. In some embodiments, the spin label is covalently attached to a cysteine residue at position 217 of the apoA-I protein. In some embodiments, the spin label is covalently attached to a cysteine residue at position 217 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 98 of the apoA-I protein. In some embodiments, the spin label is covalently attached to a cysteine residue at position 98 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 111 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 217 of the apoA-I protein. In some embodiments, the spin label is covalently attached to an amino acid at position 26, 44, 64, 98, 101, 111, 167, 217, or 226 of the apoA-I lipoprotein. In some embodiments, the native amino acid residue at position 26, 44, 64, 98, 101, 111, 167, 217, or 226 has been replaced by a cysteine residue. In some embodiments, the spin label is covalently attached to an amino acid at position 26, 44, 64, 101, 167, or 226 of the apoA-I lipoprotein. In some embodiments, the native amino acid residue at position 26, 44, 64, 101, 167, or 226 has been replaced by a cysteine residue.

In some embodiments of the invention, the spin-labeled lipoprotein probe comprises an apoA-II lipoprotein or fragment thereof, wherein the apoA-II or fragment thereof has high specificity for HDL. In some embodiments, the spin label is covalently attached to an amino acid at a single site on the apoA-II lipoprotein or fragment thereof. In some embodiments, a native amino acid residue at the single site in the apoA-II protein has been replaced by a cysteine residue.

In some embodiments of the invention, the spin-labeled lipoprotein probe comprises an apoE lipoprotein or fragment thereof, wherein the apoE or fragment thereof has high specificity for HDL. In some embodiments, the apoE lipoprotein or fragment thereof is an apoE3 lipoprotein or fragment thereof. In some embodiments, the spin label covalently attached to an amino acid at a single site on the apoE lipoprotein. In some embodiments, a native amino acid residue at the single site in the apoE protein has been replaced by a cysteine residue.

In some embodiments of the invention, the spin-labeled lipoprotein probe comprises an apoA-I mimetic, wherein the apoA-I mimetic has high specificity for HDL. In some embodiments, the apoA-I mimetic is selected from the group consisting of 18A, 18A-Pro-18A, 4F, and 4f-Pro-4F. In some embodiments, the spin label is covalently attached to a single site on the apoA-I mimetic.

In some embodiments of the invention, wherein the spin label comprises an atom that bears a free electron. In some embodiments, the atom that bears a free electron is nitrogen. In some embodiments, the spin label is (1-oxyl-2,2,5,5-tetramethyl-Δ3-pyrroline-3-methyl) methanethiosulfonate; (1-oxyl-2,2,5,5-tetramethyl-Δ3-pyrroline-3-methyl) methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; or 3-(2-iodo-acetamido-methyl)-PROXYL, free radical. In some embodiments, the spin-label is a perdeuterated spin-label. In some embodiments, the spin label is attached to an amino acid on the lipoprotein through a thiosulfonate. In some embodiments, the spin label further comprises a spacer between the spin label and the lipoprotein. In some embodiments, the spacer is methane, ethane, propane or butane.

In some embodiments, the spin-labeled lipoprotein probe binds HDL3. In some embodiments, greater than 60% of the spin-labeled lipoprotein probe binds HDL3.

In some embodiments, the composition further comprises an anti-coagulant. In some embodiments, the anti-coagulant is heparin, coumadin, warfarin, EDTA, citrate or oxalate.

In some aspects, the invention provides compositions for measuring the capacity of HDL to support reverse cholesterol transport comprising a test strip, wherein the test strip comprises a spin-labeled lipoprotein probe and a solid support, wherein the spin-labeled lipoprotein probe comprises a spin label and a protein and wherein the spin-labeled lipoprotein probe has high specificity for HDL. In some embodiments, the reverse cholesterol transport is a cholesterol efflux potential of a fluid. In some embodiments, the composition is formulated for use with a sample selected from a blood sample or a cerebral spinal fluid sample. In some embodiments, the blood sample is selected from a whole blood sample, a plasma sample, and a serum sample. In some embodiments, the sample is a mammalian blood sample. In further embodiments, the mammalian sample is a human blood sample.

In some embodiments, the test strip comprised a spin-labeled lipoprotein, wherein the spin-labeled lipoprotein probe comprises an apoA-I polypeptide or fragment thereof. In further embodiments, the apoA-I fragment comprises the HDL-binding region of apoA-I. In yet further embodiments, the spin label is covalently attached to an amino acid at a single site on the apoA-I lipoprotein or fragment thereof. In further embodiments, the spin label is covalently attached to an amino acid residue of the apoA-I lipoprotein located from residue 188 to residue 243. In yet further embodiments, the spin-label is covalently attached to an amino acid at positions 26, 44, 64, 98, 101, 111, 167, 217, or 226 of the apoA-I lipoprotein. In even further embodiments, the spin-label is covalently attached to an amino acid at positions 98, 111 or 217 of the apoA-I lipoprotein. In even further embodiments, the spin-label is covalently attached to an amino acid at positions 26, 44, 64, 101, 167 or 226 of the apoA-I lipoprotein. In yet further embodiments, the native amino acid residue at position 98, 111, or 217 have been replaced by a cysteine residue. In yet further embodiments, the native amino acid residue at position 26, 44, 64, 101, 167, or 226 has been replaced by a cysteine residue. In further embodiments, the spin label is covalently attached to a cysteine residue at position 217 of the apoA-I protein. In further embodiments, the spin label is covalently attached to a cysteine residue at position 217 of the apoA-I protein, and wherein the spin label is (1-oxyl-2,2,5,5-tetramethyl-Δ3-pyrroline-3-methyl) methanethiosulfonate. In further embodiments, the spin label is covalently attached to a cysteine residue at position 111 of the apoA-I protein, and wherein the spin label is (1-oxyl-2,2,5,5-tetramethyl-Δ3-pyrroline-3-methyl)methanethiosulfonate.

In some embodiments, the invention provides test strips comprising a spin-labeled lipoprotein probe wherein the spin-labeled lipoprotein probe comprises an apoA-II lipoprotein or fragment thereof, wherein the apoA-II or fragment thereof has high specificity for HDL. In some embodiments, the apoA-II or fragment thereof wherein 60% or more, 70% or more, 80% or more, or 90% or more of the total lipoprotein molecules associate with HDL. In further embodiments, the spin label is covalently attached to an amino acid at a single site on the apoA-II lipoprotein or fragment thereof. In further embodiments, a native amino acid residue at the single site in the apoA-II protein has been replaced by a cysteine residue.

In some embodiments, the invention provides test strips comprising a spin-labeled lipoprotein probe wherein the spin-labeled lipoprotein probe comprises an apoE lipoprotein or fragment thereof, wherein the apoE or fragment thereof has high specificity for HDL. In some embodiments, the apoE lipoprotein or fragment thereof is an apoE3 lipoprotein or fragment thereof. In further embodiments, the spin label is covalently attached to an amino acid at a single site on the apoE lipoprotein. In yet further embodiments, a native amino acid residue at the single site in the apoE protein has been replaced by a cysteine residue.

In some embodiments, the invention provides test strips comprising a spin-labeled lipoprotein probe wherein the spin-labeled lipoprotein probe comprises an apoA-I mimetic, wherein the apoA-I mimetic has high specificity for HDL. In further embodiments, the apoA-I mimetic is selected from the group consisting of 18A, 18A-Pro-18A, 4F, and 4f-Pro-4F. In further embodiments, the spin label is covalently attached to a single site on the apoA-I mimetic.

In some embodiments of any of the above embodiments, the invention provides test strips comprising a spin-labeled lipoprotein probe, wherein the spin label comprises an atom that bears a free electron. In further embodiments, the atom that bears a free electron is nitrogen. In yet further embodiments, the spin label is (1-oxyl-2,2,5,5-tetramethyl-Δ3-pyrroline-3-methyl) methanethiosulfonate; (1-oxyl-2,2,5,5-tetramethyl-Δ3-pyrroline-3-methyl) methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; or 3-(2-iodo-acetamido-methyl)-PROXYL, free radical. In further embodiments, the spin-label is a perdeuterated spin-label. In even further embodiments, the spin label is attached to an amino acid on the lipoprotein through a thiosulfonate. In further embodiments, the spin-labeled lipoprotein further comprises a spacer between the spin label and the lipoprotein. In further embodiments, the spacer is methane, ethane, propane or butane.

In some embodiments, the invention provides test strips comprising a spin-labeled lipoprotein probe wherein more than 60% of the spin-labeled lipoprotein probe binds HDL. In some embodiments, the HDL is HDL3.

In some aspects, the invention provides test strips comprising a spin-labeled lipoprotein probe and a solid support, wherein the solid support is selected from a polymer or cellulosic material with low paramagnetic properties. In some embodiments, the solid support is an adsorbent material. In some embodiments, the spin-labeled lipoprotein probe binds the adsorbent material covalently, ionically, by hydrophobic interaction, or by electrostatic interactions. In some embodiments, the adsorbent material is polyvinylidine fluoride (PVDF), nylon or nitrocellulose. In some embodiments, the solid support further comprises an adsorbent material. In some embodiments, the spin-labeled lipoprotein probe is covalently attached to the solid support. In some embodiments, the spin-labeled lipoprotein probe is covalently attached to the adsorbent material. In some embodiments, the spin-labeled lipoprotein probe is electrostatically attached to the test strip. In some embodiments, the spin-labeled lipoprotein probe is electrostatically attached to the adsorbent material. In some embodiments, the spin-labeled lipoprotein probe is attached to the test strip by hydrophobic interaction. In some embodiments, the spin-labeled lipoprotein probe is attached to the adsorbent material by hydrophobic interaction. In some embodiments, the spin-labeled lipoprotein probe is attached to the test strip by hydrophobic interaction and electrostatically. In some embodiments, the spin-labeled lipoprotein probe is attached to the adsorbent material by hydrophobic interaction and electrostatically. In some embodiments, the spin-labeled lipoprotein probe is entrapped in the adsorbent material. In some embodiments, the spin-labeled lipoprotein probe is dried onto the solid support or adsorbent material.

In some aspects, the invention provides test strips comprising spin-labeled lipoprotein probes, wherein the test strip further comprises a spin-labeled reference probe. In some embodiments, the spin-labeled reference probe is a spin-probe not affected by the presence of HDL. In some embodiments, the spin-labeled reference probe is selected from tetramethylpiperidines (TEMPO; 2,2,6,6-Tetramethylpiperidine-1-oxyl), TEMPOL (4-hydroxy-2,2,6,6-tetramethylpiperidine-1-oxyl), TAMINE (4-amino-2,2,6,6-tetramethylpiperidine-1-oxyl), BZONO (4-(benzoyloxy)-2,2,6,6-tetramethylpiperidine-1-oxyl), SLPEO (poly(ethylene oxide)-2,2,6,6-tetramethyl-piperidine-1-oxyl), and tetracyanoquinodimethane (TCNQ; 2,5-cyclohexadiene-1,4-diylidene)dimalononitrile, 7,7,8,8-tetracyanoquinodimethane).

In some embodiments of the above embodiments, the test strip comprises more than one type of spin-labeled lipoprotein probe. In some embodiments of the above embodiments, the test strip further comprises a therapeutic or therapeutic candidate. In further embodiments, the therapeutic or therapeutic candidate is a CETP inhibitor. In some embodiments, the therapeutic or therapeutic candidate is Torcetrapib, Anacetrapib, Dalcetrapib or Evacetrapib.

In some aspects, the invention provides kits for measuring by EPR an in vitro sample's capacity of HDL to support reverse cholesterol transport, the kit comprising a test strip comprising a solid support, and a spin-labeled lipoprotein probe, wherein the spin-labeled lipoprotein probe comprises a spin label and a protein and wherein the spin-labeled lipoprotein probe has high specificity for HDL.

In some aspects, the invention provides kits for testing the efficacy of a therapeutic for modulating cholesterol efflux potential, the kit comprising a test strip comprising a solid support, and a spin-labeled lipoprotein probe, wherein the spin-labeled protein probe comprises a spin label and a lipoprotein and wherein the spin-labeled lipoprotein probe has high specificity for HDL.

In some aspects, the invention provides kits for determining benefit of a therapeutic to treat hypercholesterolemia in an individual, the kit comprising a test strip comprising a solid support, a spin-labeled lipoprotein probe, and a therapeutic, wherein the spin-labeled lipoprotein probe comprises a spin label and a lipoprotein and wherein the spin-labeled lipoprotein probe has high specificity for HDL.

In some aspects, the invention provides kits for determining benefit of a therapeutic to treat Alzheimer's disease in an individual, the kit comprising a test strip comprising a solid support, a spin-labeled lipoprotein probe, and a therapeutic, wherein the spin-labeled lipoprotein probe comprises a spin label and a lipoprotein and wherein the spin-labeled lipoprotein probe has high specificity for HDL.

In some embodiments of the above aspects, the spin-labeled lipoprotein probe of the kit is present on the solid support. In some embodiments, the kits further comprising one or more additional test strips. In some embodiments, the one or more additional test strips comprise the spin-labeled lipoprotein probe at different amounts. In some embodiments, the spin-labeled lipoprotein probe is in a container separate from the test strip. In some embodiments, the container is a tube, a flatcell tube or a capillary tube. In some embodiments, the spin-labeled lipoprotein probe is provided as a dry powder.

In some embodiments of the above aspects, the kit further comprising a spin-label reference probe. In some embodiments, the spin-label reference probe is present on the solid support. In some embodiments, the spin-label reference probe is in a container separate from the test strip. In some embodiments, the spin-label reference probe is provided as a dry powder.

In some embodiments of the above aspects, the reverse cholesterol transport is a cholesterol efflux potential of a fluid. In some embodiments, the test strip of the kit is formulated for use with a sample selected from a blood sample or a cerebral spinal fluid sample. In some embodiments, the blood sample is selected from a whole blood sample, a plasma sample, and a serum sample. In some embodiments, the sample is a mammalian blood sample. In some embodiments, the mammalian sample is a human blood sample.

In some embodiments of the above aspects, the spin-labeled lipoprotein probe of the kit comprises an apoA-I polypeptide or fragment thereof. In some embodiments, the spin-labeled lipoprotein probe comprises an apoA-I fragment, wherein the apoA-I fragment comprises the HDL-binding region of apoA-I. In some embodiments, the spin label is covalently attached to an amino acid at a single site on the apoA-I lipoprotein or fragment thereof. In some embodiments, the spin label is covalently attached to an amino acid residue of the apoA-I lipoprotein located from residue 188 to residue 243. In some embodiments, the spin-label is covalently attached to an amino acid at positions 26, 44, 64, 98, 101, 111, 167, 217, or 226 of the apoA-I lipoprotein. In some embodiments, the spin-label is covalently attached to an amino acid at positions 98, 111 or 217 of the apoA-I lipoprotein. In some embodiments, the spin-label is covalently attached to an amino acid at positions 26, 44, 64, 101, 167 or 226 of the apoA-I lipoprotein. In further embodiments, the native amino acid residue at position 98, 111, or 217 has been replaced by a cysteine residue. In further embodiments, the native amino acid residue at position 26, 44, 64, 101, 167, or 226 has been replaced by a cysteine residue. In some embodiments, the spin label is covalently attached to a cysteine residue at position 217 of the apoA-I protein. In some embodiments, the spin label is covalently attached to a cysteine residue at position 217 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 111 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 26 of the apoA-I protein. In some embodiments, the spin label is covalently attached to a cysteine residue at position 26 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 44 of the apoA-I protein. In some embodiments, the spin label is covalently attached to a cysteine residue at position 44 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 64 of the apoA-I protein. In some embodiments, the spin label is covalently attached to a cysteine residue at position 64 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 101 of the apoA-I protein. In some embodiments, the spin label is covalently attached to a cysteine residue at position 101 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 167 of the apoA-I protein. In some embodiments, the spin label is covalently attached to a cysteine residue at position 167 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 226 of the apoA-I protein. In some embodiments, the spin label is covalently attached to a cysteine residue at position 226 of the apoA-I protein, and wherein the spin label is (1-oxyl-2,2,5,5-tetramethyl-Δ3-pyrroline-3-methyl)methanethiosulfonate.

In some embodiments of the aspects above, the spin-labeled lipoprotein probe of the kit comprises an apoA-II lipoprotein or fragment thereof, wherein the apoA-II or fragment thereof has high specificity for HDL. In some embodiments the apoA-II or fragment thereof wherein 60% or more, 70% or more, 80% or more, or 90% or more of the total lipoprotein molecules associate with HDL. In some embodiments, the spin label is covalently attached to an amino acid at a single site on the apoA-II lipoprotein or fragment thereof. In further embodiments, a native amino acid residue at the single site in the apoA-II protein has been replaced by a cysteine residue.

In some embodiments of the aspects above, the spin-labeled lipoprotein probe of the kit comprises an apoE lipoprotein or fragment thereof, wherein the apoE or fragment thereof has high specificity for HDL. In some embodiments, the apoE lipoprotein or fragment thereof is an apoE3 lipoprotein or fragment thereof. In some embodiments, the spin label is covalently attached to an amino acid at a single site on the apoE lipoprotein. In further embodiments, a native amino acid residue at the single site in the apoE protein has been replaced by a cysteine residue.

In some embodiments of the aspects above, the spin-labeled lipoprotein probe of the kit comprises an apoA-I mimetic, wherein the apoA-I mimetic has high specificity for HDL. In some embodiments, the apoA-I mimetic is selected from the group consisting of 18A, 18A-Pro-18A, 4F, and 4f-Pro-4F. In some embodiments, the spin label is covalently attached to a single site on the apoA-I mimetic.

In some embodiments of the above aspects, the spin label comprises an atom that bears a free electron. In some embodiments, the atom that bears a free electron is nitrogen. In some embodiments, the spin label is (1-oxyl-2,2,5,5-tetramethyl-Δ3-pyrroline-3-methyl) methanethiosulfonate; (1-oxyl-2,2,5,5-tetramethyl-Δ3-pyrroline-3-methyl) methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; or 3-(2-iodo-acetamido-methyl)-PROXYL, free radical. In some embodiments, the spin-label is a perdeuterated spin-label. In some embodiments, the spin label is attached to an amino acid on the lipoprotein through a thiosulfonate. In some embodiments, the spin-labeled lipoprotein further comprises a spacer between the spin label and the lipoprotein. In some embodiments, the spacer is methane, ethane, propane or butane. In some embodiments, more than 60% of the spin-labeled lipoprotein probe binds HDL. In some embodiments the HDL is HDL3.

In some embodiments of the above aspects, the solid support is selected from a polymer or cellulosic material with low paramagnetic properties. In some embodiments the solid support is an adsorbent material. In some embodiments, the spin-labeled lipoprotein probe binds the solid support covalently, ionically, by hydrophobic interaction, by electrostatic (charge) interactions or a combination therein. In some embodiments, the adsorbent material is polyvinylidine fluoride (PVDF), nylon or nitrocellulose. In some embodiments, the solid support further comprises an adsorbent material. In some embodiments, the spin-labeled lipoprotein probe is covalently attached to the solid support. In some embodiments, the spin-labeled lipoprotein probe is covalently attached to the adsorbent material. In some embodiments, the spin-labeled lipoprotein probe is electrostatically attached to the test strip. In some embodiments, the spin-labeled lipoprotein probe is electrostatically attached to the adsorbent material. In some embodiments, the spin-labeled lipoprotein probe is attached to the test strip by hydrophobic interaction. In some embodiments, the spin-labeled lipoprotein probe is attached to the adsorbent material by hydrophobic interaction. In some embodiments, the spin-labeled lipoprotein probe is attached to the test strip by hydrophobic interaction and electrostatically. In some embodiments, the spin-labeled lipoprotein probe is attached to the adsorbent material by hydrophobic interaction and electrostatically. In some embodiments, the spin-labeled lipoprotein probe is entrapped in the adsorbent material. In some embodiments, the spin-labeled lipoprotein probe is dried onto the solid support or adsorbent material.

In some embodiments of the above aspects, the test strip of the kit further comprises a spin-labeled reference probe. In some aspects, the spin-labeled reference probe is a spin-probe not affected by the presence of HDL. In some aspect, the spin-labeled reference probe is selected from tetramethylpiperidines (TEMPO; 2,2,6,6-Tetramethylpiperidine-1-oxyl), TEMPOL (4-hydroxy-2,2,6,6-tetramethylpiperidine-1-oxyl), TAMINE (4-amino-2,2,6,6-tetramethylpiperidine-1-oxyl), BZONO (4-(benzoyloxy)-2,2,6,6-tetramethylpiperidine-1-oxyl), SLPEO (poly(ethylene oxide)-2,2,6,6-tetramethyl-piperidine-1-oxyl), and tetracyanoquinodimethane (TCNQ; 2,5-cyclohexadiene-1,4-diylidene)dimalononitrile, 7,7,8,8-tetracyanoquinodimethane).

In some embodiments, the kit comprises more than one type of spin-labeled lipoprotein probe. In some embodiments, wherein the test strip further comprises a therapeutic or therapeutic candidate. In some embodiments, the therapeutic or therapeutic candidate is a CETP inhibitor. In some embodiments, the therapeutic or therapeutic candidate is Torcetrapib, Anacetrapib, Dalcetrapib or Evacetrapib.

In some embodiments of the above aspects, the kit further comprises a coagulant. In some embodiments, the coagulant is heparin, counadin, warfarin, EDTA, citrate or oxalae. In some embodiments, the kit further comprises instructions for use.

In some aspects, the invention provides methods for measuring capacity of high density lipoprotein (HDL) to support reverse cholesterol transport in a sample, the method comprising a) contacting an in vitro sample with a test strip comprising a solid support a spin-labeled lipoprotein probe, wherein the spin-labeled lipoprotein probe comprises a spin-label and a lipoprotein, and wherein the spin-labeled lipoprotein probe has high specificity for HDL, b) collecting the electron paramagnetic resonance (EPR) spectrum of the spin-labeled lipoprotein probe on the test strip.

In some aspects, the invention provides methods for measuring capacity of high density lipoprotein (HDL) to support reverse cholesterol transport in a sample, the method comprising a) contacting an in vitro sample with a spin-labeled lipoprotein probe, wherein the spin-labeled lipoprotein probe comprises a spin-label and a lipoprotein, and wherein the spin-labeled lipoprotein probe has high specificity for HDL b) contacting the in vitro sample with a test strip comprising a solid support, wherein a portion or all of the spin-labeled lipoprotein probe adheres to the test strip, and c) collecting the electron paramagnetic resonance (EPR) spectrum of the spin-labeled lipoprotein probe on the test strip. In some embodiments, steps a) and b) are sequential or simultaneous.

In some aspects, the invention provides methods for determining the benefit of a therapeutic to treat hypercholesterolemia or Alzheimer's disease in an individual, the method comprising a) contacting an in vitro sample from the individual with a test strip comprising a solid support and a spin-labeled lipoprotein probe, wherein the spin-labeled lipoprotein probe comprises a spin-label and a lipoprotein and wherein the spin-labeled lipoprotein probe has high specificity for HDL, b) collecting the electron paramagnetic resonance (EPR) spectrum of the spin-labeled lipoprotein probe on the test strip, wherein a decrease in the cholesterol efflux potential of the sample of the individual compared to the cholesterol efflux potential from normal individuals indicates that the individual may benefit from the therapeutic to treat hypercholesterolemia or Alzheimer's disease.

In some aspects, the invention provides methods for determining the benefit of a therapeutic to treat hypercholesterolemia or Alzheimer's disease in an individual, the method comprising a) contacting an in vitro sample from the individual with a spin-labeled lipoprotein probe, wherein the spin-labeled probe comprises a spin-label and a lipoprotein, and wherein the spin-labeled lipoprotein probe has high specificity for HDL b) contacting the in vitro sample with a test strip comprising a solid support, wherein a portion or all of the spin-labeled lipoprotein probe adheres to the test strip, c) collecting the electron paramagnetic resonance (EPR) spectrum of the spin-labeled lipoprotein probe on the test strip, wherein a decrease in the cholesterol efflux potential of the sample of the individual compared to the cholesterol efflux potential from normal individuals indicates that the individual may benefit from the therapeutic to treat hypercholesterolemia or Alzheimer's disease. In some embodiments, steps a) and b) are sequential or simultaneous.

In some aspects, the invention provides methods for optimizing the therapeutic efficacy of a therapeutic to treat hypercholesterolemia in an individual undergoing therapy to treat hypercholesterolemia, the method comprising a) contacting an in vitro sample from the individual with a test strip comprising a solid support and a spin-labeled lipoprotein probe, wherein the spin-labeled lipoprotein probe comprises a spin-label and a lipoprotein and wherein the spin-labeled lipoprotein probe has high specificity for HDL, b) collecting the electron paramagnetic resonance (EPR) spectrum of the spin-labeled lipoprotein probe on the test strip, wherein an increase in the cholesterol efflux potential of the sample of the individual compared to the cholesterol efflux potential of a sample from the individual before therapy indicates that the individual may benefit from the therapeutic to treat hypercholesterolemia. In some embodiments, therapy will be continued if an increase in cholesterol efflux potential in response to therapy is demonstrated. In some embodiments, therapy is modulated as a result of the change in cholesterol efflux potential in response to the therapy.

In some aspects, the invention provides methods for optimizing the therapeutic efficacy of a therapeutic to treat hypercholesterolemia in an individual undergoing therapy to treat hypercholesterolemia, the method comprising a) contacting an in vitro sample from the individual with a spin-labeled lipoprotein probe, wherein the spin-labeled lipoprotein probe comprises a spin-label and a lipoprotein and wherein the spin-labeled lipoprotein probe has high specificity for HDL, b) contacting the in vitro sample with a test strip comprising a solid support, wherein a portion or all the spin-labeled lipoprotein probe adheres to the test strip, c) collecting the electron paramagnetic resonance (EPR) spectrum of the spin-labeled lipoprotein probe on the test strip, wherein an increase in the cholesterol efflux potential of the sample of the individual compared to the cholesterol efflux potential of a sample from the individual before therapy indicates that the individual may benefit from the therapeutic to treat hypercholesterolemia. In some embodiments, steps a) and b) are sequential or simultaneous. In some embodiments, therapy will be continued if an increase in cholesterol efflux potential in response to therapy is demonstrated. In some embodiments, therapy is modulated as a result of the change in cholesterol efflux potential in response to the therapy.

In some aspects, the invention provides methods for diagnosing Alzheimer's disease in an individual, the method comprising a) contacting an in vitro sample from the individual with a test strip comprising a solid support and a spin-labeled lipoprotein probe, wherein the spin-labeled lipoprotein probe comprises a spin-label and a lipoprotein and wherein the spin-labeled lipoprotein probe has high specificity for HDL, b) collecting the electron paramagnetic resonance (EPR) spectrum of the spin-labeled lipoprotein probe on the test strip, wherein a decrease in the cholesterol efflux potential of the sample of the individual compared to the cholesterol efflux potential from normal individuals indicates that the individual may have Alzheimer's disease. In some embodiments, the sample is a CSF sample.

In some aspects, the invention provides methods for diagnosing Alzheimer's disease in an individual, the method comprising a) contacting an in vitro sample from the individual with a spin-labeled lipoprotein probe, wherein the spin-labeled probe comprises a spin-label and a lipoprotein, and wherein the spin-labeled lipoprotein probe has high specificity for HDL b) contacting the in vitro sample with a test strip comprising a solid support, wherein a portion or all of the spin-labeled lipoprotein probe adheres to the test strip, c) collecting the electron paramagnetic resonance (EPR) spectrum of the spin-labeled lipoprotein probe on the test strip, wherein a decrease in the cholesterol efflux potential of the sample of the individual compared to the cholesterol efflux potential from normal individuals indicates that the individual may have Alzheimer's disease. In some embodiments, steps a) and b) are sequential or simultaneous. In some embodiments, the sample is a CSF sample.

In some aspects, the invention provides methods for screening a candidate therapeutic for modulation of cholesterol efflux capacity blood of an individual, the method comprising a) contacting an in vitro sample with low cholesterol efflux capacity with a test strip comprising a solid support and a spin-labeled lipoprotein probe, wherein the spin-labeled lipoprotein probe comprises a spin-label and a lipoprotein and wherein the spin-labeled lipoprotein probe has high specificity for HDL, b) contacting the sample with the candidate therapeutic, b) collecting the electron paramagnetic resonance (EPR) spectrum of the spin-labeled lipoprotein probe on the test strip, wherein an increase in the cholesterol efflux potential of the sample indicates that the therapeutic may be useful to modulate cholesterol efflux capacity.

In some aspects, the invention provides method for screening a candidate therapeutic for modulation of cholesterol efflux capacity of an individual, the method comprising a) contacting an in vitro sample with low cholesterol efflux capacity with a spin-labeled lipoprotein probe, wherein the spin-labeled lipoprotein probe comprises a spin-label and a lipoprotein and wherein the spin-labeled lipoprotein probe has high specificity for HDL, b) contacting the in vitro sample with the candidate therapeutic, c) contacting the in vitro sample with a test strip comprising a solid support, wherein a portion or all of the spin-labeled lipoprotein probe adheres to the test strip; d) collecting the electron paramagnetic resonance (EPR) spectrum of the spin-labeled lipoprotein probe on the test strip; wherein an increase in the cholesterol efflux potential of the sample indicates that the therapeutic may be useful to modulate cholesterol efflux capacity. In some embodiments, steps a), b) and c) are sequential or simultaneous.

In some aspects the invention provides methods for determining behavioral modulators of cholesterol efflux potential, the method comprising a) contacting an in vitro sample from the individual undergoing behavioral modulation with a test strip comprising a solid support and a spin-labeled lipoprotein probe, wherein the spin-labeled lipoprotein probe comprises a spin-label and a lipoprotein and wherein the spin-labeled lipoprotein probe has high specificity for HDL, b) collecting the electron paramagnetic resonance (EPR) spectrum of the spin-labeled lipoprotein probe on the test strip, wherein an increase in the cholesterol efflux potential of the sample of the individual compared to the cholesterol efflux potential of a sample from the individual before behavioral modulation indicates that the behavioral modulation provides benefit to cholesterol efflux capacity. In some embodiments, steps a) and b) are sequential or simultaneous. In some embodiments the behavior is diet, exercise or smoking.

In some aspects, the invention provides method for determining behavioral modulators of cholesterol efflux potential, the method comprising a) contacting an in vitro sample from the individual undergoing behavioral modulation with a spin-labeled lipoprotein probe, wherein the spin-labeled lipoprotein probe comprises a spin-label and a lipoprotein and wherein the spin-labeled lipoprotein probe has high specificity for HDL; b) contacting the in vitro sample with a test strip comprising a solid support, wherein in a portion or all the spin-labeled lipoprotein probe adheres to the test strip, c) collecting the electron paramagnetic resonance (EPR) spectrum of the spin-labeled lipoprotein probe on the test strip, wherein an increase in the cholesterol efflux potential of the sample of the individual compared to the cholesterol efflux potential of a sample from the individual before behavioral modulation indicates that the behavioral modulation provides benefit to cholesterol efflux capacity. In some embodiments, steps a) and b) are sequential or simultaneous. In some embodiments the behavior is diet, exercise or smoking.

In some embodiments of the above aspects, the sample is a blood sample or a cerebral spinal fluid sample. In some embodiments, the sample is a blood sample. In some embodiments, the blood sample is selected from a whole blood sample, a plasma sample, and a serum sample. In some embodiments, the sample is a mammalian blood sample. In some embodiments, the mammalian sample is a human blood sample.

In some embodiments of the above aspects, the EPR spectrum is collected at one or more timepoints after addition of the spin-labeled lipoprotein probe to the in vitro sample. In some embodiment, the EPR spectrum is monitored at one or more of the following times after addition of the spin-labeled lipoprotein probe to the in vitro sample: 1.5 minutes, 4 minutes, 6 minutes, 8 minutes, 10 minutes, 30 minutes, 60 minutes.

In some embodiments of the above aspects, the EPR spectrum is collected at temperatures ranging from 0° C. to 37° C.

In some embodiments of the above aspects, the spin-labeled lipoprotein probe comprises an apoA-I polypeptide or fragment thereof. In some embodiments, the spin-labeled lipoprotein probe comprises an apoA-I fragment, wherein the apoA-I fragment comprises the HDL-binding region of apoA-I. In some embodiments, the spin label is covalently attached to an amino acid at a single site on the apoA-I lipoprotein or fragment thereof. In some embodiments, the spin label is covalently attached to an amino acid residue of the apoA-I lipoprotein located from residue 188 to residue 243. In some embodiments, the spin-label is covalently attached to an amino acid at positions 26, 44, 64, 98, 101, 111, 167, 217, or 226 of the apoA-I lipoprotein. In some embodiments, the spin-label is covalently attached to an amino acid at positions 98, 111 or 217 of the apoA-I lipoprotein. In some embodiments, the spin-label is covalently attached to an amino acid at positions 26, 44, 64, 101, 167 or 226 of the apoA-I lipoprotein. In further embodiments, the native amino acid residue at position 98, 111, or 217 has been replaced by a cysteine residue. In further embodiments, the native amino acid residue at position 26, 44, 64, 101, 167, or 226 has been replaced by a cysteine residue. In some embodiments, the spin label is covalently attached to a cysteine residue at position 217 of the apoA-I protein. In some embodiments, wherein the spin label is covalently attached to a cysteine residue at position 217 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 111 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 26 of the apoA-I protein. In some embodiments, wherein the spin label is covalently attached to a cysteine residue at position 26 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 44 of the apoA-I protein. In some embodiments, wherein the spin label is covalently attached to a cysteine residue at position 44 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 64 of the apoA-I protein. In some embodiments, wherein the spin label is covalently attached to a cysteine residue at position 64 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 98 of the apoA-I protein. In some embodiments, wherein the spin label is covalently attached to a cysteine residue at position 98 of the apoA-I protein, and wherein the spin label is (1-oxyl-2,2,5,5-tetramethyl-Δ3-pyrroline-3-methyl) methanethiosulfonate. In some embodiments, the spin label is covalently attached to a cysteine residue at position 101 of the apoA-I protein. In some embodiments, wherein the spin label is covalently attached to a cysteine residue at position 101 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 167 of the apoA-I protein. In some embodiments, wherein the spin label is covalently attached to a cysteine residue at position 167 of the apoA-I protein, and wherein the spin label is (1-oxyl-2,2,5,5-tetramethyl-Δ3-pyrroline-3-methyl)methanethiosulfonate. In some embodiments, the spin label is covalently attached to a cysteine residue at position 226 of the apoA-I protein. In some embodiments, wherein the spin label is covalently attached to a cysteine residue at position 226 of the apoA-I protein, and wherein the spin label is (1-oxyl-2,2,5,5-tetramethyl-Δ3-pyrroline-3-methyl)methanethiosulfonate.

In some embodiments of the above aspects, the spin-labeled lipoprotein probe comprises an apoA-II lipoprotein or fragment thereof, wherein the apoA-II or fragment thereof has high specificity for HDL. In some embodiments, the apoA-II or fragment thereof wherein 60% or more, 70% or more, 80% or more, or 90% or more of the total lipoprotein molecules associate with HDL. In some embodiments, the spin label is covalently attached to an amino acid at a single site on the apoA-II lipoprotein or fragment thereof. In further embodiments, a native amino acid residue at the single site in the apoA-II protein has been replaced by a cysteine residue.

In some embodiments of the above-aspects, the spin-labeled lipoprotein probe comprises an apoE lipoprotein or fragment thereof, wherein the apoE or fragment thereof has high specificity for HDL. In some embodiments, the apoE lipoprotein or fragment thereof is an apoE3 lipoprotein or fragment thereof. In some embodiments, the spin label is covalently attached to an amino acid at a single site on the apoE lipoprotein. In some embodiments, a native amino acid residue at the single site in the apoE protein has been replaced by a cysteine residue.

In some embodiments of the above aspects, the spin-labeled lipoprotein probe comprises an apoA-I mimetic, wherein the apoA-I mimetic has high specificity for HDL. In some embodiments, the apoA-I mimetic is selected from the group consisting of 18A, 18A-Pro-18A, 4F, and 4f-Pro-4F. In some embodiments, the spin label is covalently attached to a single site on the apoA-I mimetic.

In some embodiments of the above aspects, the spin label comprises an atom that bears a free electron. In some embodiments, the atom that bears a free electron is nitrogen. In some embodiments, the spin label is (1-oxyl-2,2,5,5-tetramethyl-Δ3-pyrroline-3-methyl) methanethiosulfonate; (1-oxyl-2,2,5,5-tetramethyl-Δ3-pyrroline-3-methyl) methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; or 3-(2-iodo-acetamido-methyl)-PROXYL, free radical. In some embodiments, the spin-label is a perdeuterated spin-label. In some embodiments, the spin label is attached to an amino acid on the lipoprotein through a thiosulfonate. In some embodiments, the spin-labeled lipoprotein further comprises a spacer between the spin label and the lipoprotein. In some embodiments, the spacer is methane, ethane, propane or butane. In some embodiments, more than 60% of the spin-labeled lipoprotein probe binds HDL. In some embodiments, the HDL is HDL3.

In some embodiments of the above aspects, the solid support is selected from a polymer or cellulosic material with low paramagnetic properties. In some embodiments, the solid support is an adsorbent material. In some embodiments, the adsorbent material is polyvinylidine fluoride (PVDF), nylon or nitrocellulose. In some embodiments, the solid support further comprises an adsorbent material. In some embodiments, the solid support further comprises an adsorbent material. In some embodiments, the spin-labeled lipoprotein probe is covalently attached to the solid support. In some embodiments, the spin-labeled lipoprotein probe is covalently attached to the adsorbent material. In some embodiments, the spin-labeled lipoprotein probe is electrostatically attached to the test strip. In some embodiments, the spin-labeled lipoprotein probe is electrostatically attached to the adsorbent material. In some embodiments, the spin-labeled lipoprotein probe is attached to the test strip by hydrophobic interaction. In some embodiments, the spin-labeled lipoprotein probe is attached to the adsorbent material by hydrophobic interaction. In some embodiments, the spin-labeled lipoprotein probe is attached to the test strip by hydrophobic interaction and electrostatically. In some embodiments, the spin-labeled lipoprotein probe is attached to the adsorbent material by hydrophobic interaction and electrostatically. In some embodiments, the spin-labeled lipoprotein probe is entrapped in the adsorbent material. In some embodiments, the spin-labeled lipoprotein probe is dried onto the solid support or adsorbent material.

In some embodiments of the above aspects, the in vitro sample further comprises an anti-coagulant. In some embodiments, the anti-coagulant is heparin, coumadin, warfarin, EDTA, citrate or oxalate.

### BRIEF DESCRIPTION OF THE DRAWINGS

FIG. 1 shows the reverse cholesterol transport pathway.

FIG. 2 shows reverse cholesterol transport in the intima. To facilitate cholesterol efflux from cholesterol laden macrophages, lipid-poor/free apoA-I binds to ABCA1. During its association with ABCA1, apoA-I acquires free cholesterol (FC) and phospholipid (PL) to form discoidal preβ HDL. These particles are acted upon by LCAT and converted to cholesterol ester core containing alpha HDL. ApoA-I is liberated from alpha HDL by the action(s) of phospholipid transfer protein (PLTP), cholesterol ester transfer protein (CETP), lipoprotein lipase (LPL) and hepatic lipase (HL) Adapted from Curtiss et al., 2006.

FIG. 3 shows distance effects on EPR spin coupling as reflected in EPR spectra.

FIG. 4 shows scanning EPR to identify structural features of a polypeptide. Spin labels were situated at number of single sites in a protein. Differences in EPR spectra reflect structural features of the protein. Adapted from Lagerstedt, J O (2007) J. Biol Chem 282:9143-9149, incorporated herein by reference.

FIG. 5 shows sampling of EPR spectra and the respective structural elements they represent (inset). The line shapes for each structural element represents the mobility of the methanethiosulfonate (MTS) spin-label. As the MTS spin-label is tethered to more ordered structures, the mobility of the spin-label is restricted in a characteristic fashion. This result is a distinctive loss of peak to peak symmetry, accompanied by broadening and flattening of the near-field and far-field peaks.

FIG. 6A is a schematic showing how spin label solvent accessibility identifies secondary structure.

FIG. 6B is a schematic showing that spin label solvent accessibility can be used to identify alpha helices and beta sheets.

FIG. 6C is a graph demonstrating that solvent accessibility of a spin label can be used to reveal structural features of a protein. A library of spin label apoA-1 molecules were made by situated at single amino acid position throughout the sequence of the protein.

FIG. 7 shows EPR spectra of apoA-I proteins with site-specifically placed spin labels and either bound to lipid or in a lipid-free environment.

FIG. 8 shows EPR analysis of HDL in plasma. Spin labeled apoA-I was added to the plasma of 4 patients to a final concentration of 0.3 mg/ml. The EPR spectra were collected at 1.5, 4, 6, 8, and 10 minutes (panel A). The spectra of lipid-free apoA-I is shown in blue. The center field amplitude of a lipid-bound apoA-I reference sample is shown as a green bar. As a frame of reference, the green bar is the same length in all panels. The data are presented in graphical form (panel B), wherein the ratio of the sample center field peak amplitude to the lipid bound reference center field amplitude (green bar, panel A) was plotted versus time.

FIG. 9 shows EPR-based analysis of apoA-I exchange. Two scenarios for exchange will be examined A) Displacement, or the measure of apoA-I leaving the rHDL particle, wherein the rHDL bears a spin labeled (dot) apoA-I (at position K133). B) Addition of apoA-I to rHDL, wherein lipid-free apoA-I is spin labeled (at position G217). By examining these two scenarios, a relative rate of displacement and insertion is determined.

FIG. 10 is a model showing apoA-I in a lipid-free environment and bound to lipid. FRET was observed in the lipid-free environment but not in when apoA-I is bound to lipid. Shown graphically in FIG. 12.

FIG. 11 shows displacement of apoA-I from rHDL. 9.6 nm POPC rHDL were generated with Alexa 350 labeled apoA-I. The rHDL were incubated at 37° C. in the presence and absence of a 5:1 ratio of lipid-free unlabeled apoA-I to rHDL apoA-I and resolved by NDGGE. After 5 hours there is a significant displacement of apoA-I from the rHDL, exhibited by the appearance of fluorescent lipid-free apoA-I. Minimal remodeling (appearance of other different sized rHDL) was observed even after 24 hours, suggesting that this reaction is an exchange of one apoA-I for another and not a product of rHDL particle remodeling. In the absence of exogenous apoA-I, no lipid free apoA-I is generated, further indicating this is a displacement reaction.

FIG. 12 is a graph showing that FRET occurs when the light emitted from an excited donor is transferred to an acceptor moiety (solid line in graph). If the donor and acceptor are beyond 75 Å apart, no FRET is observed (light shaded area in graph). The efficiency of energy transfer is measured by the amount of donor fluorescence (dark shaded region).

FIG. 13 is a graph showing the effects of oxidation kinetics of apoA-I exchange. rHDL bearing apoA-IW19:A136 were incubated in 1:5 molar ratio of unlabeled apoA-I (Trp Null apoA-I), at 37° C. for up to 6 hours. Untreated Trp Null apoA-I (shaded circles) displaced fluorescently labeled apoA-I from rHDL with τ=0.94 h. Trp Null apoA-I was oxidized by peroxynitrite and MPO. Peroxynitrite oxidation (unshaded circles) did not significantly alter apoA-I's exchange rate with τ=0.67 h. MPO oxidation of Trp Null apoA-I (black circles) created a biphasic exchange kinetics with a τ1=0.92 h and τ2=18.8 h. This is most easily explained by the presence of two apoA-I populations, an unaffected population (42.7%) and an exchange impaired population (57.3%). Maximal level of apoA-I displacement from rHDL (1:5 ratio of labeled to unlabeled at equilibrium) is indicated (dashed line; 83%). Data represent averages from 6 separate experiments.

FIG. 14 shows binding of apoA-I to human plasma. Alexa350 labeled apoA-I was added to human heparinized plasma to a concentration of 0.05, 0.1, 0.2, 0.4, and 0.8 mg/ml. The plasma with exogenous apoA-I was incubated at 37° C. for 2 hours and resolved by NDGGE. Although the gel is heavily loaded with plasma proteins, the albumin, HDL, LDL and VLDL (well bottom) regions of the gel are apparent. 5 μg of purified plasma LDL and HDL were run as controls. The fluorescent signal for apoA-I appears in the HDL fraction but not the albumin, LDL or VLDL.

FIG. 15A shows site directed spin-labeling of apoA-I. Cysteine substitutions are engineered into apoA-I at locations where it is desired to incorporate a stable nitroxide radical spin-label. ApoA-I cysteine substitution mutants are incubated at RT (30 min) with nitroxide linked MTS, which specifically reacts with the sulfhydril group of the cysteine residue to incorporate the spin-label at the site of cysteine substitution.

FIG. 15B shows the effect of lipid-binding on EPR spectra. Residues within apoA-I respond to differing degrees to the presence of lipid. Position A176 is not significantly altered by lipid, whereas position G217 is dramatically affected. This difference (arrows) can be exploited to serve as a measure of HDL binding.

FIG. 16 shows EPR spectroscopy of mouse plasma. Top panel is the spectra of a spin-labeled apoA-I at 4° C. and 37° C. where the spin label was located at residue 217. The middle panel is a graph showing the change in signal over time for the sample in the top panel. The bottom panel is the spectra of a spin-labeled apoA-I at 4° C. and 37° C. where the spin label was located at residue 111.

FIG. 17 is a graph showing the percent response of binding of a spin-labeled lipoprotein probe to HDL in plasma from C57Bl/6 mice and CH3 mice.

FIG. 18 shows EPR spectral position for monitoring apoA-I binding to HDL.

FIG. 19 shows ApoA-I binding to HDL in human plasma.

FIG. 20 shows traces of apoA-I binding to HDL in control human plasma samples.

FIG. 21 is a graph showing the results of an HDL function assay in plasma from nine individuals whose diabetic/metabolic syndrome status had been identified.

### DETAILED DESCRIPTION OF THE INVENTION

The invention provides compositions and methods for measuring the capacity of high density lipoprotein (HDL) to support reverse cholesterol transport in blood, the method comprising a) adding a spin-labeled lipoprotein probe with high specificity for HDL to an in vitro blood sample, and b) collecting the electron paramagnetic resonance (EPR) spectrum of the sample. The EPR spectrum is used to assess the extent and/or rate of binding of the lipoprotein to the HDL which correlates to the capacity of the HDL to support reverse cholesterol transport. As such, the methods of the invention may be used to identify individuals at risk for cardiovascular diseases such as coronary artery disease, stroke and peripheral vascular disease. The methods of the invention may be used to identify individuals with diabetes or at risk for diabetes (e.g. in a pre-diabetic state). The methods of the invention may also be used to identify individuals at risk for Alzheimer's disease or as a diagnosis for early stages of Alzheimer's disease. Compositions and kits for use in the determination of the capacity of high density lipoprotein (HDL) to support to support reverse cholesterol transport in blood or cereberal spinal fluid (CSF) are also provided.

The invention is based in part on the unexpected discovery that EPR spectroscopy can be used to detect changes in the structure of apoA-I as it binds to HDL in an in vitro blood sample. As shown in the examples herein, EPR spectroscopy has been successfully shown to measure structural changes in apoA-I upon binding to HDL in an in vitro blood sample and can correlate to the cholesterol efflux capacity of the HDL present in the in vitro blood sample. The methods of the invention may therefore be used to identify individuals with reduced cholesterol efflux capacity, even for certain individuals whose lipid panels (e.g. levels of HDL, LDL, VLDL obtained by routine blood tests appear normal. These EPR methods may also be used in the determination of the capacity of HDL to support reverse cholesterol transport in CSF and to identify individuals with reduced cholesterol efflux capacity of CSF.

In some aspects, the invention provides methods of determining the risk for developing cardiovascular disease in an individual, wherein the reverse cholesterol transport capacity of HDL in blood from the individual is measured by adding a spin-labeled lipoprotein probe with high specificity for HDL to an in vitro blood sample from the individual and the EPR spectrum of the spin-labeled lipoprotein probe in the in vitro blood sample is collected. The collected EPR spectrum is then compared to one or more negative controls and/or one or more positive controls. The negative control may be the EPR spectrum of a lipid-free or lipid-poor spin-labeled lipoprotein probe, where the spin label and lipoprotein are the same as the spin label and lipoprotein forming the spin-labeled lipoprotein probe. The positive control may be the EPR spectrum of a spin-labeled lipoprotein probe bound to lipid, such as dimyristoylphosphatidyl choline, or may be one or more historical spectra of spin-labeled lipoprotein probes bound to HDL in in vitro blood samples from individuals not at risk for cardiovascular disease (where the spin label and lipoprotein are the same as the spin label and lipoprotein forming the spin-labeled lipoprotein probe). In some embodiments, the positive control is a sample derived from a conglomerate or a single sample for an individual or a group of individuals identified as low risk for cardiovascular disease and the cholesterol efflux potential of the sample is determined to be high by alternative means (i.e. cell-based cholesterol efflux assays). A decrease in the reverse cholesterol transport capacity of the HDL in blood from the individual compared to positive control(s) may indicate a risk for cardiovascular disease. In some embodiments, the individual is a human at risk for cardiovascular disease. In some embodiments the human at risk for cardiovascular disease is diabetic. In some embodiments, the methods of the invention are used to determine if the human has diabetes or is at risk of developing diabetes. In some embodiments the human at risk for cardiovascular disease is obese. In some embodiments, the human at risk from cardiovascular disease suffers from dyslipidemia. In some embodiments, the human at risk for cardiovascular disease has a family history of cardiovascular disease.

In some aspects, the invention provides methods of monitoring the course of therapy for cardiovascular disease in an individual wherein the reverse cholesterol transport capacity of HDL in blood from the individual is measured by adding a spin-labeled lipoprotein probe with high specificity for HDL to an in vitro blood sample from the individual and the EPR spectrum of the spin-labeled lipoprotein probe is collected, where the spin-labeled lipoprotein probe has high specificity for HDL. The reverse cholesterol transport capacity of HDL in blood from the individual undergoing therapy for vascular disease is monitored over time during the course of the therapy. In some embodiments, the reverse cholesterol transport capacity of HDL in blood from the individual is measured by prior to the onset of therapy. In some embodiments, the reverse cholesterol transport capacity of HDL in blood from the individual is measured before, during and/or after therapy. In some embodiments the individual is a mammal. In some embodiments the individual is a human. In some embodiments, the individual is a non-human mammal. In some embodiments, the cardiovascular disease is coronary artery disease, atherosclerosis, peripheral vascular disease or stroke.

In some aspects, the invention provides methods for evaluating known or potential therapeutics for cardiovascular disease, wherein the reverse cholesterol transport capacity of HDL in blood from a test animal is measured by adding a spin-labeled lipoprotein probe with high specificity for HDL to an in vitro blood sample from the test animal and the EPR spectrum of the spin-labeled lipoprotein probe is in the in vitro blood sample is collected, wherein the test animal has been subjected to the therapy. An increase in reverse cholesterol transport capacity is indicative of therapeutic efficacy. In some embodiments, the test animal is a mouse, a rat, a rabbit, a hamster, a guinea pig, a dog, a cat or a pig. In some embodiments, the test animal is a non-human primate. In some embodiments the therapy includes administration of one or more pharmaceutical agents. In some embodiments the therapy includes changes in diet and/or the level of physical activity. In some embodiments the therapy may include administration of one or more pharmaceutical agents in combination with changes in diet and/or the level of physical activity. The reverse cholesterol transport capacity of HDL in blood from the test animal undergoing therapy is monitored over time during the course of the therapy. In some embodiments, the reverse cholesterol transport capacity of HDL in blood from the test animal is measured prior to the onset of therapy. In some embodiments, the reverse cholesterol transport capacity of HDL in blood from the test animal is measured before, during and/or after therapy.

In some aspects, the invention provides, methods for determining efficacy of a known or potential therapy for cardiovascular disease, the method comprising, a) determining the reverse cholesterol transport capacity of an in vitro blood sample from a test individual according to any of the above embodiments, wherein the therapeutic has been added to the blood sample after removal from the individual and prior to analysis. In some embodiments, the test therapeutic is added to multiple blood samples at different concentrations. In some embodiments, the blood is incubated with the test therapeutic for various amounts of time; for example but not limited to 1 min, 2 min, 3 min, 4 min, 5 min, 6 min, 7 min, 8 min, 9 min, 10 min., or greater than 10 min. In a further embodiment of the embodiments above, an increase in the reverse transport capacity of the in vitro blood sample from the test animal is indicative of therapeutic efficacy. In some embodiments, the test individual is a non-human mammal (e.g., mouse, a rat, a rabbit, a hamster, a guinea pig, a dog, a cat or a pig). In some embodiments, the test animal is a non-human primate.

In some aspects, the invention provides kits for measuring the reverse cholesterol transport capacity of HDL in in vitro blood samples. In some embodiments, the kit comprises a spin-labeled lipoprotein probe with high specificity for HDL. In some embodiments, the spin-labeled lipoprotein probe is added to an in vitro blood sample and analyzed by EPR. In some embodiments, the kit is used to determine the risk for developing cardiovascular disease in an individual. In some embodiments, the individual at risk for cardiovascular disease has one of more of the following risk factors: diabetes, obesity, hypertension or smoking. In some embodiments, the kit is used to detect diabetes in an individual. In some embodiments, the kit is used to monitor the course of treatment for cardiovascular disease. In some embodiments, the kit is used to measure the therapeutic efficacy of known or potential therapies for cardiovascular disease in animal models of cardiovascular diseases.

In some aspects, the invention provides kits for measuring the reverse cholesterol transport capacity of HDL in CSF samples. In some embodiments, the kit comprises a spin-labeled lipoprotein probe with high specificity for HDL. In some embodiments, the spin-labeled lipoprotein probe is added to CSF sample and analyzed by EPR. In some embodiments, the kit is used to determine the risk for developing Alzheimer's disease in an individual. In some embodiments, the kit is used to monitor the course of treatment for Alzheimer's disease. In some embodiments, the kit is used to measure the therapeutic efficacy of known or potential therapies for Alzheimer's in animal models of Alzheimer's diseases.

In some aspects, the invention provides compositions comprising a spin-labeled lipoprotein probe with high specificity for HDL. In some embodiments, the lipoprotein is an apoA-I mimetic. In some embodiments, the lipoprotein is apoA-I and the spin label is (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; or (+)-(1-oxyl-2,2,5,5-tetramethylpyrrolidin-3-yl)methyl methanesulfonate. In some embodiments, the invention provides a composition comprising a spin-labeled lipoprotein probe with high specificity for HDL in an in vitro blood sample.

In some aspects, the invention provides the use of a solid substrate such as cellulose or plastic polymer. The strip can either be impregnated with the EPR probe prior to addition of sample (e.g., blood plasma, CSF, etc.) or a mixture of EPR probe and sample (e.g., blood plasma, CSF, etc.) are brought into contact with the strip. The strip may be impregnated with a known quantity of an EPR reference standard that has a spectrum unique to the EPR spin probe. This standard is used to calibrate the EPR instrument. After addition of sample (e.g., plasma, CSF, etc.) or sample/probe to the test strip, it is allowed to react and is inserted into an EPR instrument for collection of the spectrum. The instrument will measure the EPR spectral properties of the spin-labeled lipoprotein probe (e.g., apoA-I EPR spin probe). The differential spectral properties of the spin-labeled lipoprotein probe (e.g., apoA-I EPR spin) in the presence of plasma versus phosphate buffered saline, pH 7.4 gives a measure of HDL function (e.g., reverse cholesterol transport capacity).

In some aspects, the invention provides containers comprising spin-labeled lipoprotein probes for the measuring the capacity of HDL to support reverse cholesterol transport in blood or spinal fluid. In some embodiments, the container is a tube, a flatcell tube or a capillary tube. In some embodiments, the spin-labeled lipoprotein probe in the container is in the form of a dry powder. In some embodiments, the spin-labeled lipoprotein probe in the container is lyophilized. In some embodiments, the spin-labeled lipoprotein probe in the container is formulated for use with a fluid sample such as a blood sample, a serum sample, a plasma sample, a cerebral spinal fluid sample. In some embodiments, the fluid sample may be added to the spin-labeled lipoprotein probe in the container. In some embodiments, the EPR spectra of the spin-labeled lipoprotein probe, with or without the fluid sample, can be obtained from the container. In some embodiments, the container is in a form that can be used with an EPR spectrometer. In some embodiments, the container comprising the spin-labeled lipoprotein probe is in a kit as described herein.

Unless defined otherwise, the meanings of all technical and scientific terms used herein are those commonly understood by one of skill in the art to which this invention belongs. Singleton, et al., Dictionary of Microbiology and Molecular Biology, 3rd ed., John Wiley and Sons, New York (2002), and Hale & Marham, The Harper Collins Dictionary of Biology, Harper Perennial, N.Y. (1991) provide one of skill with a general dictionary of many of the terms used in this invention. It is to be understood that this invention is not limited to the particular methodology, protocols, and reagents described, as these may vary. One of skill in the art will also appreciate that any methods and materials similar or equivalent to those described herein can also be used to practice or test the invention.

“High density lipoprotein” or “HDL” is a circulating, non-covalent assembly of amphipathic proteins that enable lipids like cholesterol and triglycerides to be transported within the water-based bloodstream. HDL is comprised of ˜50% by mass amphipathic proteins that stabilize lipid emulsions composed of a phospholipid monolayer (˜25%) embedded with free cholesterol (˜4%) and a core of triglycerides (˜3%) and cholesterol esters (˜12%). Subclasses of HDL include HDL2 and HDL3. HDL2 particles are larger and contain a higher content of lipid whereas HDL3 particles are smaller and contain less lipid. Further subclasses include from largest particle to smallest particle, HDL2b, HDL2a, HDL3a, HDL3b, and HDL3c.

“HDL-C” refers to cholesterol in HDL particles. The concentration of HDL-C refers to the concentration of cholesterol in humans carried on HDL.

As used herein, “dysfunctional HDL” refers to HDL with reduced capacity for reverse cholesterol transport. In some examples, dysfunctional HDL refers to HDL with reduced cholesterol efflux compared to the cholesterol efflux of HDL from a healthy individual not at risk for cardiovascular disease.

“Reverse cholesterol transport” (RCT) is a process whereby excess cholesterol is transported from peripheral tissues to the liver or steroidogenic tissues. The reverse cholesterol transport pathway is generally considered to have three main steps: (i) cholesterol efflux, the initial removal of cholesterol from various pools of peripheral cells; (ii) cholesterol esterification by the action of lecithin:cholesterol acyltransferase (LCAT), thereby preventing a re-entry of effluxed cholesterol into cells; and (iii) uptake of the cholesteryl ester by HDL and delivery of the HDL-cholesteryl ester complex to liver cells.

“Cholesterol efflux potential” is the ability of HDL to promote reverse cholesterol transport by accepting cholesterol from lipid-laden tissue, such as macrophages. Decreases in cholesterol efflux potential in CSF may be indicative of Alzheimer's disease or risk of developing Alzheimer's disease.

“Electron paramagnetic resonance (EPR) spectroscopy” is a spectroscopic technique that detects chemical species that have unpaired electrons. EPR is also known as “electron spin resonance” (ESR) or “electron magnetic resonance” (EMR), and these terms may be used interchangeably. EPR is process of resonant absorption of microwave radiation by paramagnetic ions or molecules, with at least one unpaired electron spin, and in the presence of a static magnetic field. By application of a strong magnetic field to material containing paramagnetic species, the individual magnetic moment arising via the electron “spin” of the unpaired electron can be oriented either parallel or anti-parallel to the applied field. This creates distinct energy levels for the unpaired electrons, making it possible for net absorption of electromagnetic radiation (in the form of microwaves) to occur. The resonance condition takes place when the magnetic field and the microwave frequency are such that the energy of the microwaves corresponds to the energy difference of the pair of involved spin states.

A “spin label” is an organic molecule which possesses an unpaired electron. In some examples, the spin label has the ability to bind to another molecule; for example, a protein. Spin labels may be used as tools for probing proteins or biological membrane local dynamics using EPR spectroscopy. Site-directed spin labeling allows one to monitor a specific region within a protein; for example, in protein structure examinations, amino acid-specific spin label can be used.

As used herein, a “spin-labeled lipoprotein probe” is a lipoprotein that comprises at least one spin-label. The spin-labeled lipoprotein probe has high specificity for HDL. In some examples, the spin label may be situated at a single site on the lipoprotein, for example, at a single amino acid residue. In some examples, the spin-labeled lipoprotein probe associates with an HDL particle. In some examples, the spin-labeled lipoprotein probe may freely exchange with a lipoprotein in an HDL particle. Exchange is based on lipid and particle affinity. A protein with higher or equivalent affinity can displace another protein of equal or less affinity. As used herein, the lipoprotein portion of the spin-labeled lipoprotein is not limited to proteins to which one or more lipid molecules are attached. In general, the lipoprotein portion of the spin-labeled lipoprotein probe has the capacity to associate with lipid. In addition, the lipoprotein portion of the spin-labeled lipoprotein is not limited to full-length proteins but encompassed polypeptides and peptides and the like.

As used herein, a “lipoprotein” refers to a group of proteins to which one or more lipid molecules is attached or is capable of being attached. In some cases, a lipoprotein may be a “lipid-poor lipoprotein” in which four or fewer molecules of phospholipid are bound. As used herein, a lipoprotein includes a protein to which no lipid is attached but which can be exchanged in an HDL particle (e.g. an apolipoprotein).

As used herein “equilibrium binding” refers to a state where the rate of association of one molecule to another is equal to the rate of dissociation of the two molecules. In some examples, equilibrium binding can be determined by monitoring the binding of two molecules over time; for example, by monitoring EPR spectra over time. Equilibrium binding may be achieved when the percentage of molecules bound remains at an approximate steady state. As used herein, the “transition temperature” or a lipid is the temperature in which the lipid transitions, or melts, from a solid or gel phase to a liquid phase.

As used herein, “sample” refers to a portion of a larger whole to be tested. A sample includes but is not limited to a body fluid such as blood, cerebral spinal fluid, urine, saliva, and the like.

As used herein, “blood sample” refers to refers to a whole blood sample or a plasma or serum fraction derived therefrom. In some examples, the in vitro blood sample refers to a human blood sample such as whole blood or a plasma or serum fraction derived therefrom. In some examples, the in vitro blood sample refers to a non-human mammalian (“animal”) blood sample such as whole blood or a plasma or serum fraction derived therefrom. The blood sample may also be from a test animal (e.g., an animal used in in vivo experiments of pharmaceutical agent efficacy or toxicity), a pet, livestock, etc. As used herein the term “whole blood” refers to a blood sample that has not been fractionated and contains both cellular and fluid components.

As used herein, “whole blood” refers to freshly drawn blood or a conventionally-drawn blood sample which may optionally contain an anticoagulant. In some examples, the whole blood may be drawn into a vacutainer the whole blood may also be from a test animal (e.g., an animal used in in vivo experiments of pharmaceutical agent efficacy or toxicity), a pet, livestock, etc.

As used herein, “plasma” refers to the fluid, non-cellular component of the whole blood. Depending on the separation method used, plasma may be completely free of cellular components, or may contain various amounts of platelets and/or a small amount of other cellular components. Because plasma includes various clotting factors such as fibrinogen, the term “plasma” is distinguished from “serum” as set forth below.

As used herein, the term “serum” refers to whole mammalian serum, such as, for example, whole human serum, whole serum derived from a test animal, whole serum derived from a pet, whole serum derived from livestock, etc. Further, as used herein, “serum” refers to blood plasma from which clotting factors (e.g., fibrinogen) have been removed.

As used herein, the term “cerebral spinal fluid” or “CSF” refers to mammalian cerebral spinal fluid, such as, for example, human cerebral spinal fluid. CSF is a bodily fluid that occupies the subarachnoid space and the ventricular system around and inside the brain and spinal cord. Cerebral spinal fluid may be drawn from the brain or spinal fluid. The term also encompasses CSF from non-human mammals e.g. mouse, rat, rabbit, dog, pig, non-human primates and the like.

As used herein, the term therapeutic is used for any compound that has or may have a therapeutic effect. Examples of therapeutics include but are not limited to small molecules, proteins, peptides, antibodies, nucleic acids, lipids, carbohydrates. As used herein, a compound undergoing testing for a potential therapeutic effect is considered a therapeutic; for example an experimental therapeutic or an experimental drug.

The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms polypeptide and protein also encompass fragments of full-length polypeptide or protein, unless clearly indicated otherwise by context.

As used herein, “apoA-I” refers to a lipoprotein that is a major component of HDL. An example of an apoA-I protein is the human apoA-I protein (e.g. NM_{—}000039.1). Other examples of a human apoA-I protein are the ApoA-1^{Milano} protein and the apoA-I^{Iowa} protein. The term also encompasses apoA-I proteins from non-human mammals e.g. mouse, rat, rabbit, dog, pig, non-human primates and the like. Also encompassed by the term “apoA-I” are homologues of apoA-I.

As used herein, “apoA-II” refers to a lipoprotein that is the second most abundant component of HDL. An example of an apoA-II protein is the human apoA-II protein (e.g. NP_{—}001634) protein. The term also encompasses apoA-II proteins from non-human mammals e.g. mouse, rat, rabbit, dog, pig non-human primates and the like.

As used herein, “apoE” refers to a lipoprotein that is involved in lipid metabolism and cholesterol transport. An example of an apoE protein is the human apoE protein (e.g. NM_{—}000041.2) protein. There are three isoforms of the human apoE protein, ApoE2, ApoE3, ApoE4. ApoE3 is the predominant form of apoE, whereas apoE2 and apoE4 display distinct distributions among the lipoprotein particles (HDL, LDL, VLDL). The term also encompasses apoE proteins from non-human mammals e.g. mouse, rat, rabbit, dog, pig, non-human primates and the like.

As used herein, a protein “mimetic” is a peptide-containing molecule that mimics elements of a protein secondary structure. A protein mimetic is expected to permit molecular interactions similar to the natural molecule. For example, some apoA-I mimetics mimic the HDL-binding property of the parent apoA-I protein (Garber, D W et al. (1992) Arterioscler Thromb Vasc Biol 12:886-894; Wool, G D et al. (2009) J Lipid Res 50:1889-1900). In some embodiments the apoA-I mimetic is a mimetic of a non-human mammalian apoA-I protein. In some embodiments the apoA-I mimetic is a mimetic of human apoA-I protein.

For use herein, unless clearly indicated otherwise, use of the terms “a”, “an,” and the like refers to one or more.

Reference to “about” a value or parameter herein includes (and describes) embodiments that are directed to that value or parameter per se. For example, description referring to “about X” includes description of “X.” Numeric ranges are inclusive of the numbers defining the range.

It is understood that aspects and embodiments of the invention described herein include “comprising,” “consisting,” and “consisting essentially of” aspects and embodiments.

### HDL and Reverse Cholesterol Transport

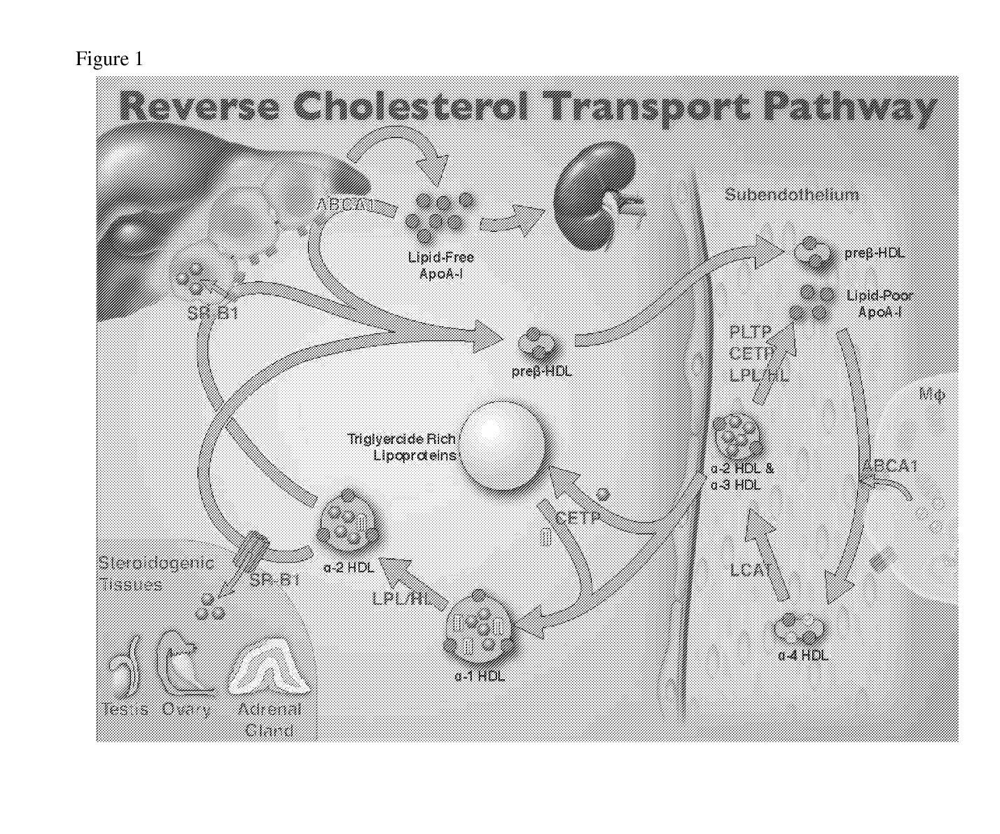

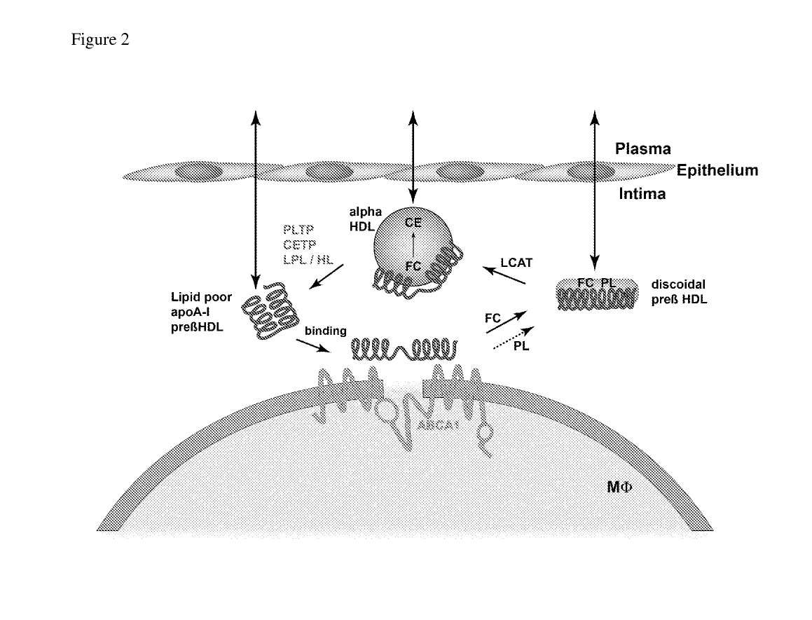

The anti-atherogenic property of HDL is in large part attributed to its role in RCT, the process by which excess cholesterol is transported from peripheral tissues to the liver or steroidogenic tissues [40, 41]. In plasma, the vast majority of apoA-I is associated with spherical HDL, a complex of apolipoproteins, phospholipids, triglycerides (TG), free cholesterol and cholesterol esters [42]. However, the primary acceptor of cholesterol and phospholipids from macrophages is lipid-free or lipid-poor apoA-I (containing up to 4 phospholipid molecules) [43], which is the preferred substrate of ABCA1 [44-49], the primary mediator of cholesterol efflux. Because apoA-I is predominantly synthesized in the liver, the most likely source of lipid-free apoA-I in the intima are apoA-I molecules that are displaced from HDL. Mounting evidence supports the notion that the production of lipid-free/lipid-poor apoA-I from mature HDL and its re-lipidation by ABCA1 is an ongoing process in the arterial wall (FIGS. 1 and 2) that is critical for maintenance of endothelial health and cholesterol balance in macrophages. To facilitate cholesterol efflux from cholesterol laden macrophages, lipid-poor/free apoA-I binds to ABCA1. During its association with ABCA1, apoA-I acquires free cholesterol (FC) and phospholipid (PL) to form discoidal preβ HDL. These particles are acted upon by LCAT and converted to cholesterol ester core-containing alpha HDL. ApoA-I is liberated from alpha HDL by the action(s) of phospholipid transfer protein (PLTP), cholesterol ester transfer protein (CETP), lipoprotein lipase (LPL) and hepatic lipase (HL).

### Electron Paramagnetic Resonance

Electron paramagnetic resonance is the study of the resonant response to microwave- or radio-frequency radiation of paramagnetic materials placed in a magnetic field. Paramagnetic substances normally have an odd number of electrons or unpaired electrons, but in some cases, EPR is observed for ions or biradicals with an even number of electrons. By application of a strong magnetic field to material containing paramagnetic species, the individual magnetic moment arising via the electron “spin” of the unpaired electron can be oriented either parallel or anti-parallel to the applied field. This creates distinct energy levels for the unpaired electrons, making it possible for net absorption of electromagnetic radiation (in the form of microwaves) to occur. The situation referred to as the resonance condition takes place when the energy of the microwaves corresponds to the energy difference ΔE of the pair of involved spin states.

To overcome the intrinsic low sensitivity of the magnetic dipole transitions responsible for EPR, samples are placed in resonant cavities. Typically spectra are collected in the steady state at the X-band microwave frequency of approximately 9 gigahertz, by slowly sweeping the magnetic field through resonance. Free electrons resonate in a magnetic field of 3250 gauss (325 millitesla) at the microwave frequency of 9.1081 GHz, whereas organic free radicals resonate at slightly different magnetic fields characteristic of each particular molecule. Although X-band microwaves are the most common, EPR spectrometers are available for other frequencies; for example, the frequencies listed in Table 1.

TABLE 1
Microwave bands

| Designation | n/GHz | l/cm | B(electron)/Tesla |
|---|---|---|---|
| S | 3.0 | 10.0 | 0.107 |
| X | 9.5 | 3.15 | 0.339 |
| K | 23 | 1.30 | 0.82 |
| Q | 35 | 0.86 | 1.25 |
| W | 95 | 0.315 | 3.3 |

Microwaves reflected back from the cavity (less when power is being absorbed) are routed to the diode detector, and any power reflected from the diode is absorbed completely by the Load. The diode is mounted along the E-vector of the plane-polarized microwaves and thus produces a current proportional to the microwave power reflected from the cavity. In principle, the absorption of microwaves by the sample could be detected by noting a decrease in current in the microammeter but in practice, such a direct current (d.c.) measurement would be far too noisy to be useful.

The solution to the signal-to-noise ratio problem is to introduce small amplitude field modulation. An oscillating magnetic field is super-imposed on the d.c. field by means of small coils, usually built into the cavity walls. When the field is in the vicinity of a resonance line, it is swept back and forth through part of the line, leading to an alternating current (a.c.) component in the diode current. This a.c. component is amplified using a frequency selective amplifier, thus eliminating a great deal of noise. The modulation amplitude is normally less than the line width. Thus the detected a.c. signal is proportional to the change in sample absorption. Spectra are plotted as detected signal versus magnetic field.

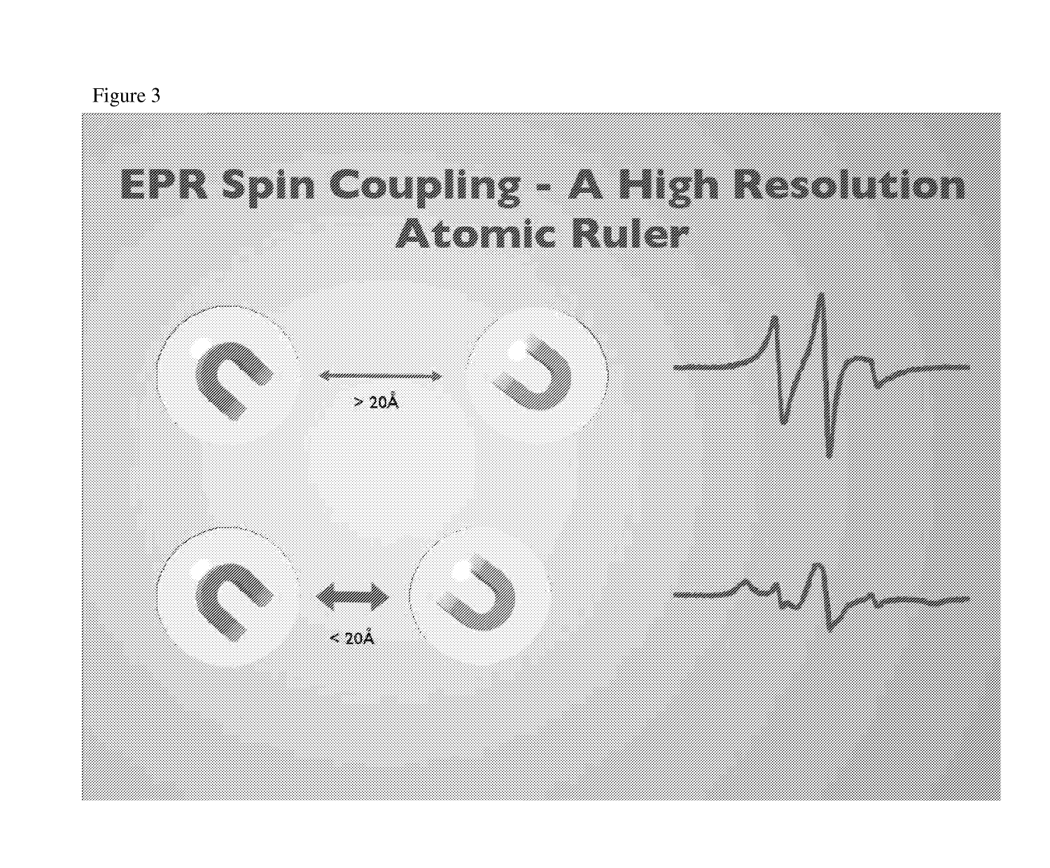

Applications of EPR in chemistry include characterization of free radicals, studies of organic reactions, and investigations of the electronic properties of paramagnetic inorganic molecules. Information obtained is used in the investigation of molecular structure. EPR is used widely in biology in the study of metal proteins, for nitroxide spin labeling, and in the investigation of radicals produced during reaction processes in proteins and other biomacromolecules. EPR reports the structural environment (regional flexibility and solvent accessibility) and the interaction distances between spin labels (FIG. 3)

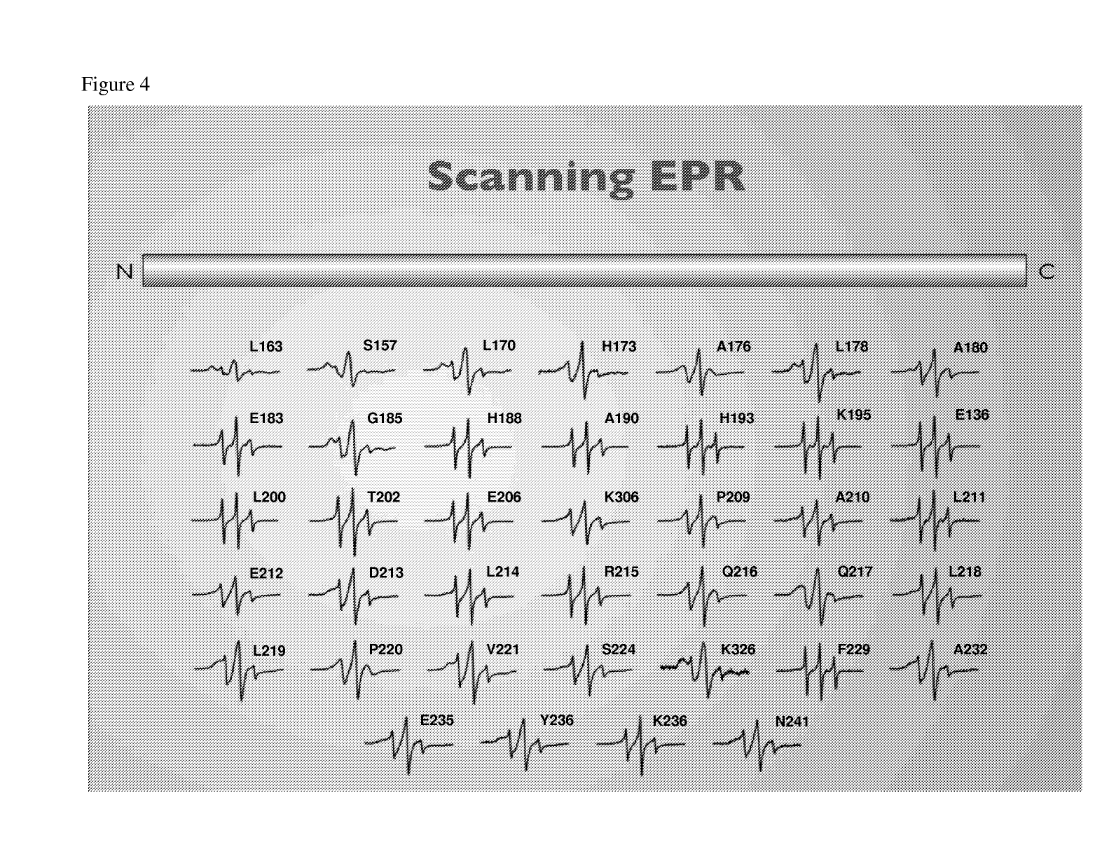

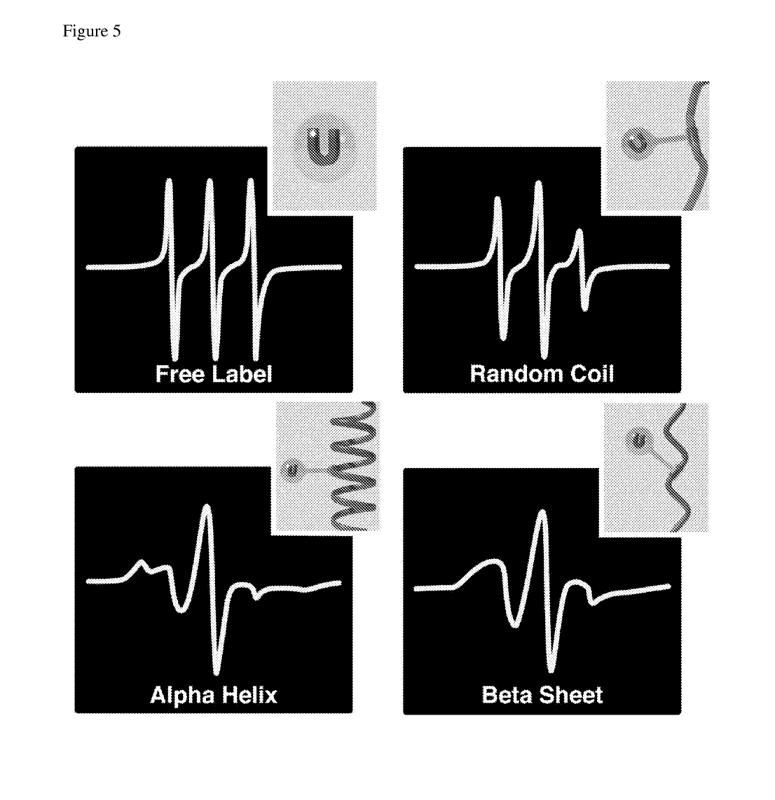

Examples of EPR spectra of spin-labeled lipoproteins and the respective structural elements they represent (inset) are presented in FIGS. 4 and 5. Due to a hyperfine interaction with the nitrogen nuclear spin, the nitroxide spin label spectrum contains three peaks from left to right; a near-field peak, a center peak and a far field peak. The line shape (width) of the three resonant peaks is dependent on the orientation of the hyperfine element within the lab magnetic field. Motional averaging of the hyperfine element is reflected in the shape of each EPR peak (line), such that spin label motions that occur on the time scale of 10⁻¹⁰ to 10⁻⁶ sec influence the spectral line widths. As the spin label is tethered to more ordered structures, the mobility of the spin label is restricted in a characteristic fashion. The result is a distinctive loss of peak to peak symmetry, accompanied by broadening and flattening of the near-field and far-field peaks.

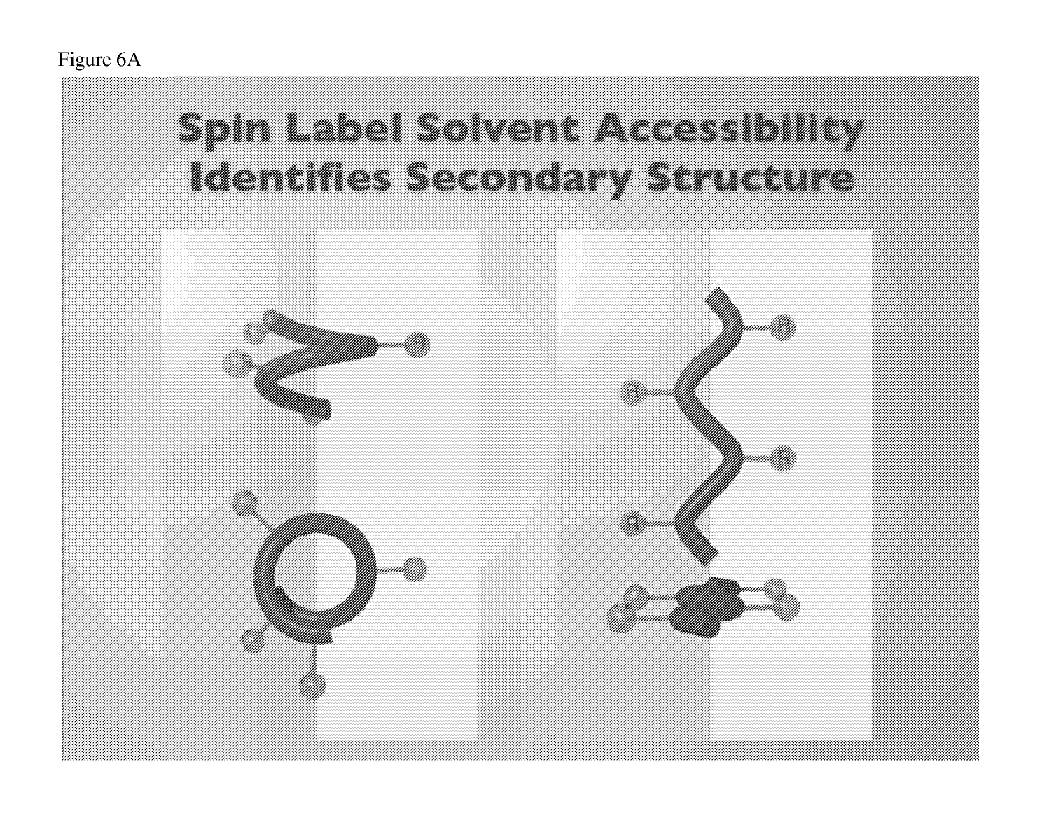

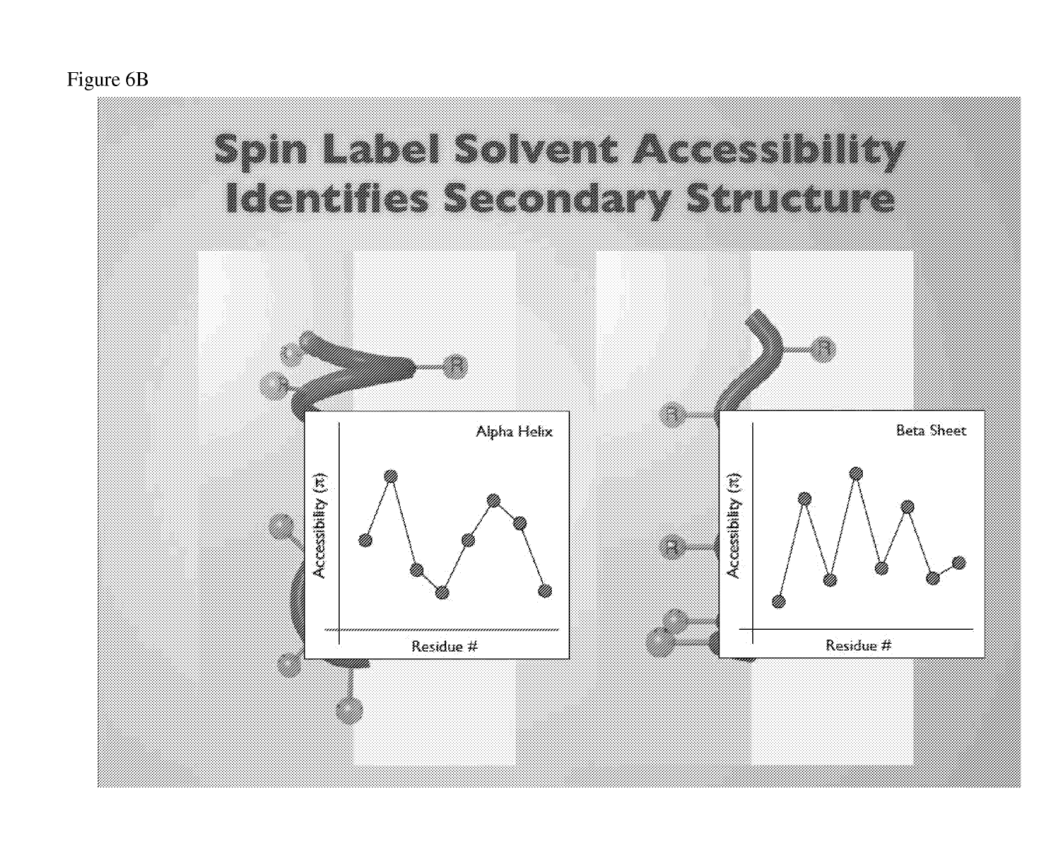

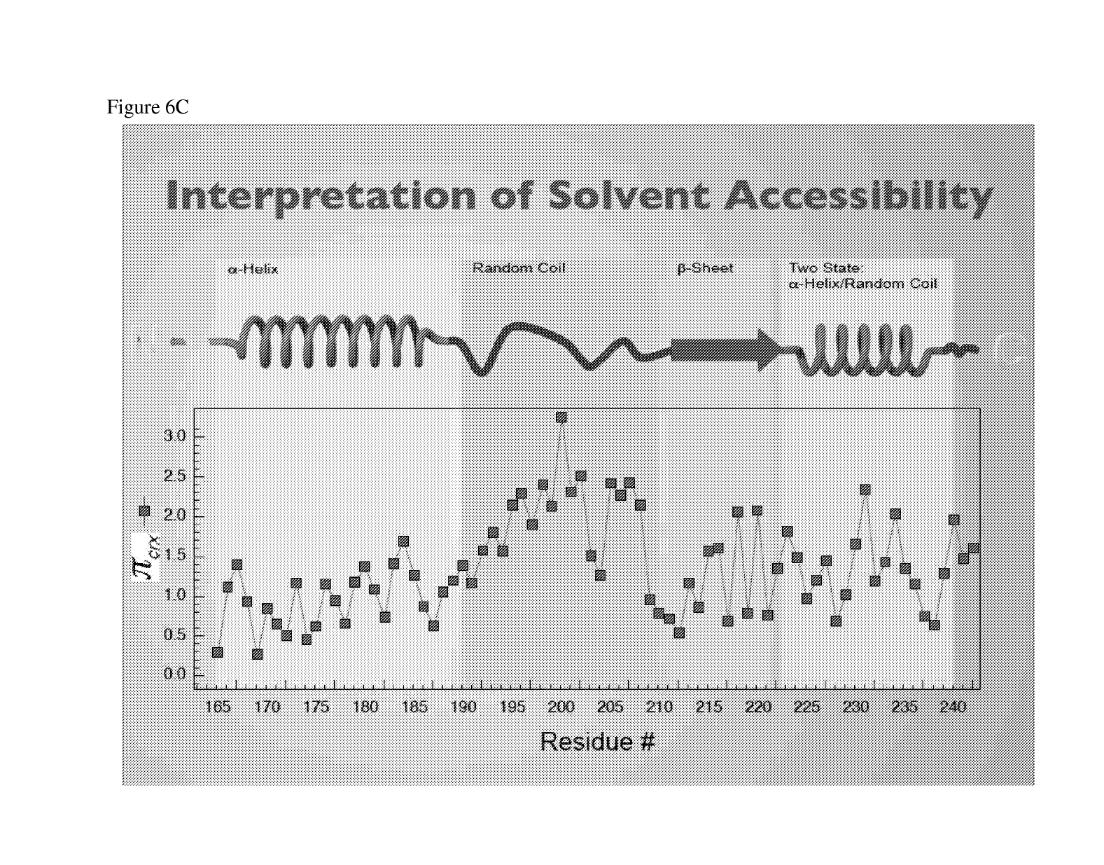

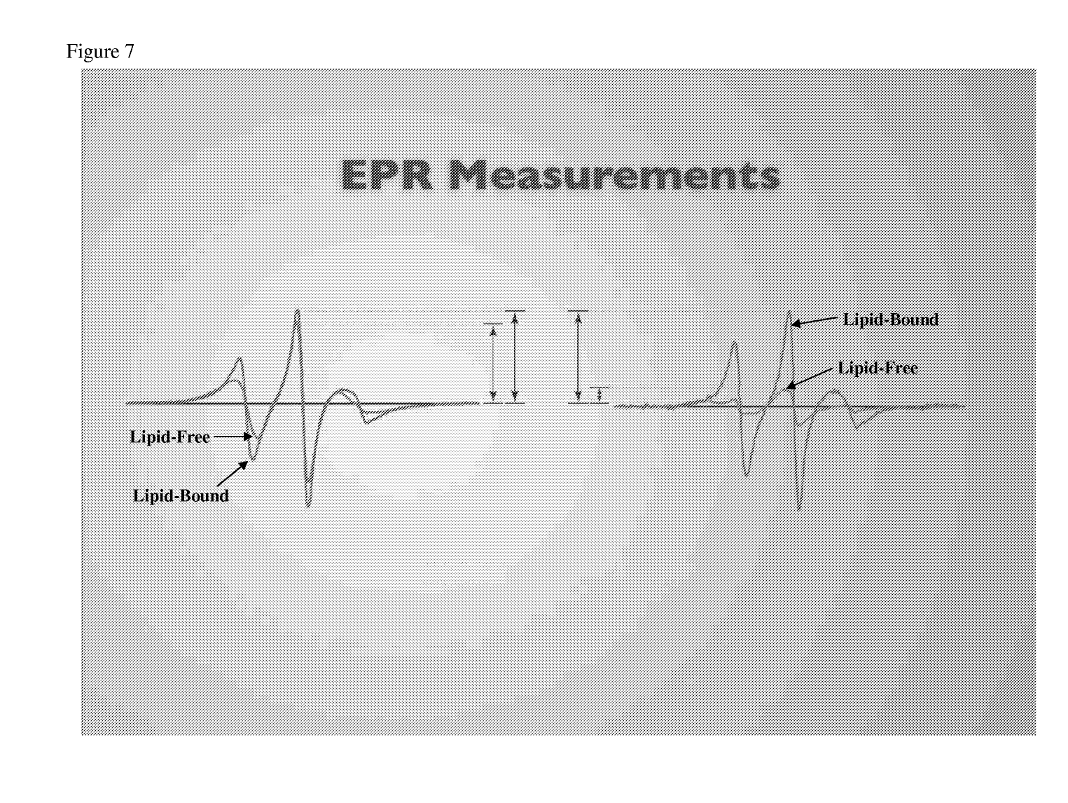

In some embodiments of the invention, EPR is employed as a means of examining apoA-I structure. Using EPR, the structure of apoA-I in lipid-free or lipid-poor and lipid-bound states has been examined, for example, the EPR solution to apoA-I's N-terminal structure on 9.6 nm reconstituted discoidal HDL [61, 65]. Specifically, the conformation of regions/domains targeted with nitroxide spin labels can be derived from three principal parameters measurable by EPR: side chain mobility of the tethered spin label and its local peptide backbone dynamics (FIG. 5), solvent accessibility of the spin-label, and the proximity of nearby (<22 Å for continuous wave EPR as employed here) spins whose dipolar coupling can identify tertiary and quaternary structural elements. Hubbell and co-workers have characterized modulations in EPR spectral line-shapes and have identified specific protein structural characteristics associated with these changes [73, 74]. The line shapes for each structural element represents the mobility of the spin-label; for example as the spin label is tethered to more ordered structures, the mobility of the spin label is restricted in a characteristic fashion. This result in a distinctive loss of peak to peak symmetry, accompanied by broadening and flattening of the near-field and far-field peaks. Likewise, dipolar coupling among nearby spins results in a distinctive spectral broadening (that can evaluated independently from broadening due to motional restriction, see ref [61]). Thus EPR spectral changes arising for changes in the dipolar coupling (i.e., spatial proximity of the labels) can also be exploited to detect conformational rearrangements in the protein as reported spin labels targeted to specific domains. (FIG. 6) Thus, as illustrated above, from this type of analysis of EPR spectra one can reliably draw structural conclusions from the shape of the EPR spectra of spin-labeled sites in proteins. Therefore, if a spin label is positioned in portion of apoA-I that bears a unique conformation in the lipid-free/lipid-poor versus lipid bound state, the EPR spectra can be used to distinguish between these two forms of apoA-I or other spin-labeled lipoprotein probe utilized (FIG. 7).

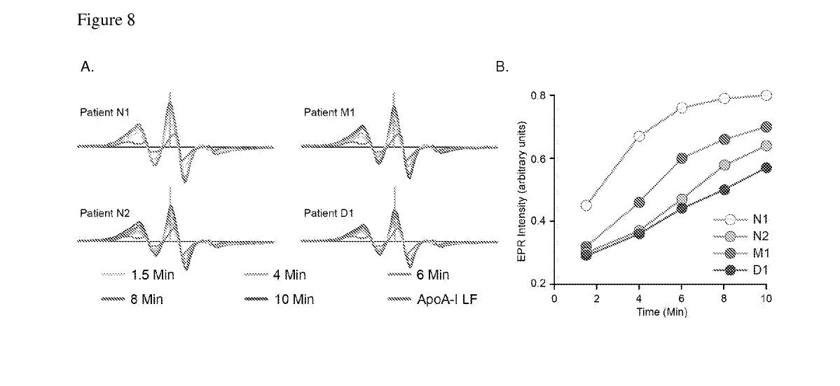

In some embodiments of the invention, the EPR spectra of spin-labeled lipoprotein probes with high specificity for HDL in in vitro blood samples are quantitated by measuring the amplitude of the center peak of the spectra, which is a function of the peak's line width. The amplitude of the center peak is the distance between the baseline and the greatest signal above the baseline (See for example FIG. 8). In some embodiments, the EPR spectra are quantitated by measuring a change in the line width of the center peak. In some embodiments, the EPR spectra are quantitated by measuring the width between a center line of the center peak and the point where the spectrum returns to the baseline. In some embodiments, the EPR spectra are quantitated by measuring the ratio of the amplitude of the center peak to the amplitude of the near-field peak and/or the far-field peak.

In some embodiments of the invention, a change in binding of a spin-labeled lipoprotein to HDL in an in vitro blood sample is measured by comparing the EPR spectrum of a spin-labeled lipoprotein probe with high specificity for HDL in the in vitro blood sample with the EPR spectrum of negative and positive controls. In some embodiments, the embodiments, the change in binding of a spin-labeled lipoprotein to HDL in an in vitro blood sample is measured by comparing the center peak amplitude of the EPR spectrum of a spin-labeled lipoprotein probe in the in vitro blood sample with the center peak amplitude of the EPR spectrum of a spin-labeled lipoprotein probe bound to lipid and/or the center peak amplitude of the EPR spectrum of a lipid-poor spin-labeled lipoprotein probe. In some embodiments, the change in binding of a spin-labeled lipoprotein to HDL in an in vitro blood sample is measured by comparing the width of the center peak of the EPR spectrum of a spin-labeled lipoprotein probe with high specificity for HDL in the in vitro blood sample with the width of the center peak of the EPR spectrum of a spin-labeled lipoprotein probe with high specificity for HDL bound to lipid and/or the width of the center peak of the EPR spectrum of a lipid-poor spin-labeled lipoprotein probe. In some embodiments, the change in binding of a spin-labeled lipoprotein to HDL in an in vitro blood sample is measured by comparing the ratio of the amplitude of the center peak to the amplitude of the near-field peak and/or the far-field peak of the EPR spectrum with the ratio of the amplitude of the center peak to the amplitude of the near-field peak and/or the far-field peak of the EPR spectrum of a spin-labeled lipoprotein probe bound to lipid and/or the ratio of the amplitude of the center peak to the amplitude of the near-field peak and/or the far-field peak of the EPR spectrum of a lipid-poor spin-labeled lipoprotein probe.

In some embodiments of the invention, a change in binding of a spin-labeled lipoprotein to HDL in an in vitro blood sample is measured by comparing the EPR resonance spectra of a spin-labeled lipoprotein probe with high specificity for HDL in the in vitro blood sample with the resonance of the spin label in the EPR spectra of a spin-labeled lipoprotein probe bound to lipid and/or the resonance of the nitroxide in the EPR spectra of a lipid-poor spin-labeled lipoprotein probe. The resonance of the spin label may be determined by the frequency of the center peak along the X axis (magnetic field) of the spectrum.

Quantitative EPR is described in Eaton, G R et al (Quantiative EPR, SpringerWien New York (2010))

### Lipoproteins

The invention provides methods of measuring the reverse cholesterol transport capacity of HDL in an in vitro blood sample by collecting the EPR spectra of a spin-labeled lipoprotein probe with high specificity for HDL. The methods are based in part on the ability of the spin-labeled lipoprotein probe to exchange with lipoproteins in the HDL particle. In some embodiments of the invention, the lipoprotein with high specificity for HDL is a lipoprotein where 60% or more, 70% or more, 80% or more or 90% or more of the total lipoprotein molecules associate with HDL. In some embodiments, a lipoprotein with high specificity for HDL is a lipoprotein where less than or about 40%, 30%, 20% or 10% associate with low density lipoproteins (VLD) or very low density lipoproteins (VLDL). In some embodiments, the lipoprotein is not an apoE4 protein.

Spin-labeled lipoprotein probes are designed such that the EPR spectrum of the spin-labeled lipoprotein probe with high specificity for HDL when bound to lipid is different than the EPR spectrum of the same spin-labeled lipoprotein probe when in a lipid-poor environment. An EPR spectrum of the spin-labeled lipoprotein probe in an in vitro blood sample indicates whether the spin-labeled lipoprotein probe associates with the HDL present in the sample. An EPR spectrum of the spin-labeled lipoprotein probe with high specificity for HDL in an in vitro blood sample that more closely resembles the EPR spectrum of the spin-labeled lipoprotein probe with high specificity for HDL bound to lipid indicates that the spin-labeled lipoprotein probe is associated with the HDL. An EPR spectrum of the spin-labeled lipoprotein probe with high specificity for HDL in an in vitro blood sample that more closely resembles the EPR spectrum of the same spin-labeled lipoprotein probe with high specificity for HDL in a lipid-poor environment indicates that the spin-labeled lipoprotein probe did not associate with the HDL in the sample. Association of the spin-labeled probe with HDL in the in vitro blood sample correlates with the reverse cholesterol transport capacity of the HDL in the in vitro blood sample. Higher levels of binding of the spin-labeled lipoprotein probe to the HDL indicate higher capacity for reverse cholesterol transport.

Apolipoproteins generally possess a class A amphipathic act-helix structural motif (Segrest et al. (1994) Adv. Protein Chem. 45:303-369), and/or a b-sheet motif. Apolipoproteins generally include a high content of α-helix secondary structure with the ability to bind to hydrophobic surfaces. A characteristic feature of these proteins is their ability to interact with certain lipid bilayer vesicles and to transform them into disc-shaped complexes (for a review, see Narayanaswami and Ryan (2000) Biochimica et Biophysica Acta 1483:15-36). Upon contact with lipids, the protein undergoes a conformational change, adapting its structure to accommodate lipid interaction.

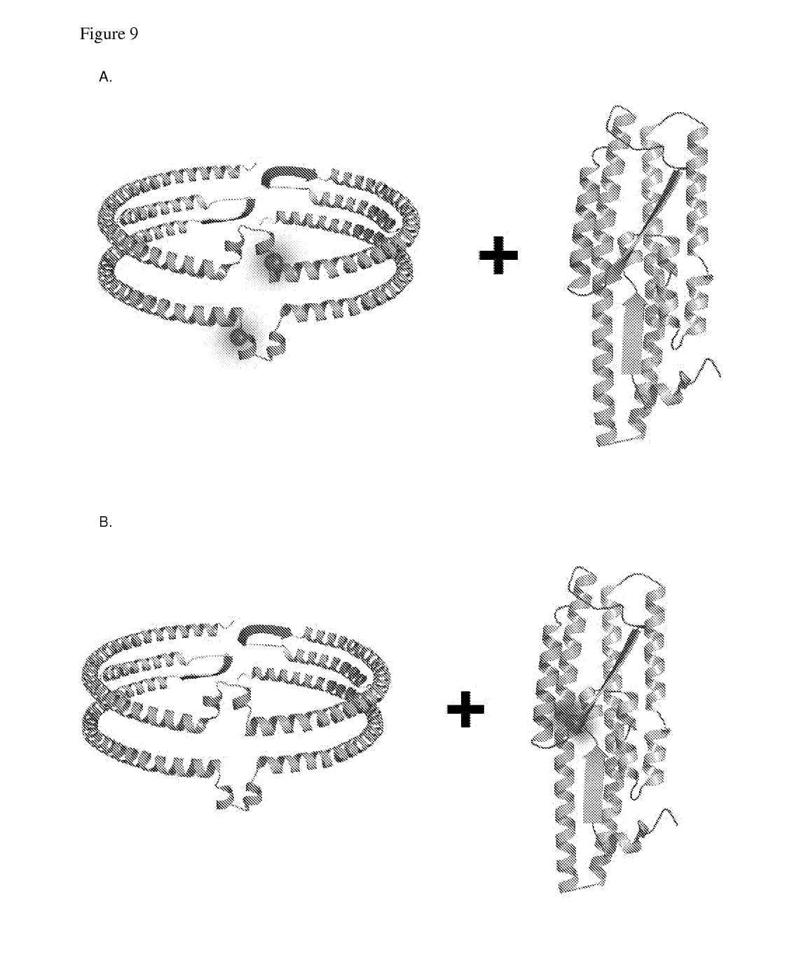

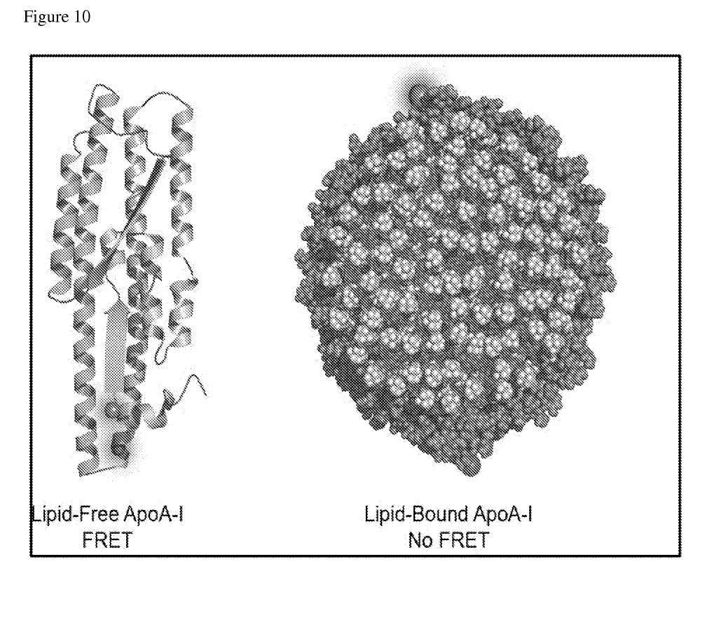

In some embodiments of the invention, the spin-labeled lipoprotein probe with high specificity for HDL is a spin-labeled apoA-I protein or fragment thereof. ApoA-I is the major component of HDL. In plasma, the vast majority (98% for normal humans) of apoA-I associates with spherical HDL. The primary acceptor of cholesterol and phospholipid from peripheral tissues, however, is lipid-free or lipid-poor apoA-I, which is the preferred substrate of the plasma membrane transporter ATP-binding cassette A1 (ABCA1). In the absence of lipids apoA-I can assume a compact 4-helical bundle (FIG. 9) (Cavigiolio, G et al. (2010) J. Biol. Chem. 285:18847-18857). Upon lipidation (association with lipid), the amphipathic α-helices substitute protein-protein contact for protein-lipid interaction corresponding to an opening of the helical bundles into an extended belt-like α-helix, which wraps around the perimeter of the nascent HDL particle FIG. 10.

In some embodiments, the apoA-I is a human apoA-I; for example, the apoA-I is a human apoA-I with an amino acid sequence set forth in GenBank Accession No. NM_{—}000039.1.

The sequence of the human apoA-I protein is:

| (SEQ ID NO: 1) | |
|---|---|
| −24 | mkaavltlav lfltgsqarh fwqqdeppqs pwdrvkdlat vyvdvlkdsg rdyvsqfegs |
| 37 | algkqlnlkl ldnwdsvtst fsklreqlgp vtqefwdnle keteglrqem skdleevkak |
| 97 | vqpylddfqk kwqeemelyr qkveplrael qegarqklhe lqeklsplge emrdrarahv |
| 157 | dalrthlapy sdelrqrlaa rlealkengg arlaeyhaka tehlstlsek akpaledlrq |
| 217 | gllpvlesfk vsflsaleey tkklntq |

In some embodiments, the spin-labeled lipoprotein probe with high specificity for HDL comprises a fragment of apoA-I. In some embodiments, the apoA-I is an apoA-I peptide. In some embodiments, the apoA-I fragment comprises residues 188 to 243 of the apoA-I protein. In some embodiments, the apoA-I fragment consists of residues 188 to 243 of the apoA-I protein. In some embodiments, the apoA-I fragment comprises residues 220-241 of the apoA-I protein. In some embodiments, the apoA-I fragment consists of residues 220-241 of the apoA-I protein. In some embodiments, the apoA-I fragment comprises residues 61-67 of the apoA-I protein. In some embodiments, the apoA-I fragment consists of residues 61-67 of the apoA-I protein. In some embodiments, the apoA-I fragment comprises residues 83-91 of the apoA-I protein. In some embodiments, the apoA-I fragment consists of residues 83-91 of the apoA-I protein. In some embodiments, the apoA-I fragment comprises residues 96-103 of the apoA-I protein. In some embodiments, the apoA-I fragment consists of residues 96-103 of the apoA-I protein. In some embodiments, the apoA-I fragment comprises residues 116-124 of the apoA-I protein. In some embodiments, the apoA-I fragment consists of residues 116-124 of the apoA-I protein. In some embodiments, the apoA-I fragment comprises residues 139-146 of the apoA-I protein. In some embodiments, the apoA-I fragment consists of residues 139-146 of the apoA-I protein. In some embodiments, the apoA-I fragment comprises residues 162-169 of the apoA-I protein. In some embodiments, the apoA-I fragment consists of residues 162-169 of the apoA-I protein. In some embodiments, the apoA-I fragment comprises residues 182-190 of the apoA-I protein. In some embodiments, the apoA-I fragment consists of residues 182-190 of the apoA-I protein. In some embodiments, the apoA-I fragment comprises residues 204-212 of the apoA-I protein. In some embodiments, the apoA-I fragment consists of residues 204-212 of the apoA-I protein. In some embodiments, the apoA-I fragment comprises residues 216-221 of the apoA-I protein. In some embodiments, the apoA-I fragment consists of residues 216-221 of the apoA-I protein. In some embodiments, the apoA-I fragment comprises residues 1-186 of the apoA-I protein. In some embodiments, the apoA-I fragment consists of residues 1-186 of the apoA-I protein.

In some embodiments, the spin-labeled lipoprotein probe with high specificity for HDL comprises a fragment of apoA-I produced by proteolytic cleavage of the apoA-I protein. In some embodiments, the apoA-I fragment is produced by digesting apoA-I with chymotrypsin. In some embodiments, the apoA-I chymotryptic fragment comprises residues 1-229 of the apoA-I protein. In some embodiments, the apoA-I chymotryptic fragment comprises residues 1-192 of the apoA-I protein. In some embodiments, the apoA-I chymotryptic fragment comprises residues 19-243 of the apoA-I protein. In some embodiments, the apoA-I chymotryptic fragment comprises residues 58-243 of the apoA-I protein. In some embodiments, the apoA-I chymotryptic fragment comprises residues 1-223 of the apoA-I protein. In some embodiments, the apoA-I chymotryptic fragment comprises residues 1-212 of the apoA-I protein. In some embodiments, the apoA-I chymotryptic fragment comprises residues 1-35-243 of the apoA-I protein.

In some embodiments of the invention, the spin-labeled lipoprotein probe with high specificity for HDL comprises a fragment of apoA-I wherein the fragment comprises a structural domain of apoA-I. In some embodiments, the apoA-I fragment comprises the α-helix domain of the apoA-I protein. In some embodiments, the apoA-I fragment comprises the random coil domain of the apoA-I protein. In some embodiments, the apoA-I fragment comprises the β-sheet domain of the apoA-I fragment. In some embodiments, the apoA-I fragment comprises the two state α-helix/random coil domain of the apoA-I protein.

In some embodiments, the spin label is located at a single residue on the apoA-I protein or fragment thereof. In some embodiments, the apoA-I probe comprises two spin-labels, each at a single amino acid residue in the apoA-I protein. In some embodiments, the spin label is covalently attached to the apoA-I protein or fragment thereof. In some embodiments, the spin label is non-covalently attached or associated with the apoA-I protein or fragment thereof. In some embodiments the spin label is attached to a cysteine residue in the apoA-I protein. The native apoA-I protein does not contain a cysteine residue. In some embodiments of the invention, the apoA-I is engineered to contain a cysteine residue by replacing a native amino acid residue with a cysteine residue. This provides a means for specifically directing the spin label to a single site on the apoA-I protein with a reduced risk of generating a spin-labeled apoA-I protein in which a portion of the spin-labels are attached to the apoA-I protein in a random fashion. In some embodiments of the invention, the apoA-I protein is engineered to locate single cysteine residue at any site from residue 188 to residue 243. In some embodiments, the spin label is attached to the single cysteine residue genetically engineered at any site from residue 188 to residue 243. In some embodiments, the spin label is attached to a residue of apoA-I at any site from residue 188 to residue 243. In some embodiments of the invention, the spin label is attached to a cysteine genetically engineered to sites 98, 111 or 217 of the apoA-I protein. In some embodiments of the invention, the spin label is attached to a residue of the apoA-I protein to sites 98, 111 or 217 of the apoA-I protein. In some embodiments, the spin label is attached to residue 217 of the apoA-I protein. In some embodiments, the spin label is attached to a cysteine residue genetically engineered to site 217 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 217 of the apoA-I protein (SEQ ID NO:2). In some embodiments, the spin label is covalently attached to a cysteine residue at position 217 of the apoA-I protein. In some embodiments, the spin label is attached to an amino acid at position 26, 44, 64, 101, 167, or 226 of the apoA-I lipoprotein. In some embodiments, the native amino acid residue at position 26, 44, 64, 101, 167, or 226 has been replaced by a cysteine residue. In some embodiments, the spin label is attached to residue 26 of the apoA-I protein. In some embodiments, the spin label is attached to a cysteine residue genetically engineered to site 26 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl) methyl methanesulfonate and is covalently attached to a cysteine residue genetically engineered to position 26 of the apoA-I protein (SEQ ID NO:2). In some embodiments, the spin label is attached to residue 44 of the apoA-I protein. In some embodiments, the spin label is attached to a cysteine residue genetically engineered to site 44 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 44 of the apoA-I protein (SEQ ID NO:2). In some embodiments, the spin label is attached to residue 64 of the apoA-I protein. In some embodiments, the spin label is attached to a cysteine residue genetically engineered to site 64 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 64 of the apoA-I protein (SEQ ID NO:2). In some embodiments, the spin label is attached to residue 101 of the apoA-I protein. In some embodiments, the spin label is attached to a cysteine residue genetically engineered to site 101 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 101 of the apoA-I protein (SEQ ID NO:2). In some embodiments, the spin label is attached to residue 111 of the apoA-I protein. In some embodiments, the spin label is attached to a cysteine residue genetically engineered to site 111 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 111 of the apoA-I protein (SEQ ID NO:2). In some embodiments, the spin label is attached to residue 167 of the apoA-I protein. In some embodiments, the spin label is attached to a cysteine residue genetically engineered to site 167 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 167 of the apoA-I protein (SEQ ID NO:2). In some embodiments, the spin label is attached to residue 226 of the apoA-I protein. In some embodiments, the spin label is attached to a cysteine residue genetically engineered to site 226 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 226 of the apoA-I protein (SEQ ID NO:2).

The sequence of the apoA-I protein genetically engineered to have a cysteine residue at position 217 is as follows.

| (SEQ ID NO: 2) | |
|---|---|
| −24 | mkaavltlav lfltgsqarh fwqqdeppqs pwdrvkdlat vyvdvlkdsg rdyvsqfegs |
| 37 | algkqlnlkl ldnwdsvtst fsklreqlgp vtqefwdnle keteglrqem skdleevkak |
| 97 | vqpylddfqk kwqeemelyr qkveplrael qegarqklhe lqeklsplge emrdrarahv |
| 157 | dalrthlapy sdelrqrlaa rlealkengg arlaeyhaka tehlstlsek akpaledlrq |
| 217 | glcpvlesfk vsflsaleey tkklntq |

In some embodiments of the invention, the spin label is located in the α-helix domain of the apoA-I protein. The α-helix domain is not static. In lipid-free apoA-I, α-helix domain includes positions 8-14, 30-40, 51-85, 92-137, 146-187, 200-210, and 223-239. In some embodiments, the spin label is located in the random coil domain of the apoA-I protein. In some embodiments, the spin label is located in the β-sheet domain of the apoA-I fragment. In some embodiments, the spin label is located in the two state α-helix/random coil domain of the apoA-I protein.

In some embodiments of the invention, the spin-labeled lipoprotein probe with high specificity for HDL is a spin-labeled apoA-II protein or fragment thereof. ApoA-II is the second most abundant lipoprotein component of HDL. The protein is found in plasma as a monomer, homodimer or heterodimer with apolipoprotein D. Defects in this gene may result in apolipoprotein A-II deficiency or hypercholesterolemia. In some aspects of the invention, the apoA-II protein is human apoA-II protein. An example of a human apoA-II amino acid sequence is as follows:

| (SEQ ID NO: 3) | |
|---|---|
| 1 | mkllaatvll lticslegal vrrqakepcv eslvsqyfqt vtdygkdlme kvkspelqae |
| 61 | aksyfekske qltplikkag telvnflsyf velgtqpatq |

In some embodiments, the spin label is located at a single residue on the apoA-II protein or fragment thereof. In some embodiments, the spin label is covalently attached to the apoA-II protein or fragment thereof. In some embodiments, the spin label is non-covalently attached or associated with the apoA-II protein or fragment thereof. In some embodiments of the invention, the apoA-II protein or fragment thereof comprises two spin-labels, each at a single amino acid residue in the apoA-II protein. In some embodiments the spin label is attached to a cysteine residue in the apoA-II protein or fragment thereof. The native apoA-II protein contains one cysteine residue located in the signal peptide. The mature apoA-II protein does not contain a cysteine residue. In some embodiments of the invention, the mature apoA-II protein is engineered to locate single cysteine residue at any site from residue 24 to residue 100. In some embodiments of the invention, the apoA-II precursor is engineered to replace the native cysteine residue in the signal peptide with another amino acid residue and engineered to contain another cysteine residue by replacing a native amino acid residue with a cysteine residue. In some embodiments, the spin label is attached to the engineered cysteine residue of the apoA-II protein.

In some embodiments of the invention, the spin-labeled lipoprotein probe with high specificity for HDL is a spin-labeled apoE protein or fragment thereof. ApoE is essential for the normal catabolism of triglyceride-rich lipoprotein constituents. In some aspects of the invention, the apoE protein is human apoE protein. There are three isoforms of the human apoE protein, ApoE2, ApoE3, ApoE4. ApoE3 is the predominant form of apoE whereas apoE2 and apoE4 are associated with different distributions among the lipoprotein particles. In some embodiments, the spin label is attached to the engineered cysteine residue of the apoE protein. In some embodiments, the apoE protein is an apoE3 protein. In some embodiments, the apoE protein is not an apoE4 protein. In some embodiments, the apoE protein is an apoE2 protein. In some embodiments, the apoE protein is not an apoE2 protein or an apoE4 protein.

An example of a human apoE amino acid sequence is as follows:

| (SEQ ID NO: 4) | |
|---|---|
| 1 | mkvlwaallv tflagcqakv eqavetepep elrqqtewqs gqrwelalgr fwdylrwvqt |
| 61 | lseqvqeell ssqvtqelra lmdetmkelk aykseleeql tpvaeetrar lskelqaaqa |
| 121 | rlgadmedvc grlvqyrgev qamlgqstee lrvrlashlr klrkrllrda ddlqkrlavy |
| 181 | qagaregaer glsairerlg plveqgrvra atvgslagqp lqeraqawge rlrarmeemg |
| 241 | srtrdrldev keqvaevrak leeqaqqirl qaeafqarlk swfeplvedm qrqwaglvek |
| 301 | vqaavgtsaa pvpsdnh |

In some embodiments, the spin label is located at a single residue on the apoE protein or fragment thereof. In some embodiments, the spin label is covalently attached to the apoE protein or fragment thereof. In some embodiments, the spin label is non-covalently attached or associated with the apoE protein or fragment thereof. In some embodiments of the invention, the apoE protein or fragment thereof comprises two spin-labels, each at a single amino acid residue in the apoE protein. In some embodiments the spin label is attached to a cysteine residue in the apoE protein or fragment thereof. The native apoE protein contains two cysteine residues, one located in the signal peptide and one located in the mature apoE protein. In some embodiments of the invention, the apoE protein is engineered to replace the native cysteine residues and engineered to contain another cysteine residue by replacing a native amino acid residue with a cysteine residue.

In some embodiments of the invention, the spin-labeled lipoprotein probe with high specificity for HDL comprises a mimetic of a lipoprotein. In some embodiments, the mimetic of a lipoprotein is a mimetic of apoA-I. In some embodiments the apoA-I mimetic is a mimetic of a non-human mammalian apoA-I protein. In some embodiments the apoA-I mimetic is a mimetic of human apoA-I protein. In some embodiments, the apoA-I mimetic is 18A, 18A-Pro-18A, 4F and 4f-Pro-4F. ApoA-I mimetic 18A is made of the sequence DWLKAFYDKVAEKLKEAF (SEQ ID NO: 5) (Garber, D W et al. (1992) Arteriosclerosis, Thrombosis, and Vascular Biology 12:886-894). Mimetic 18A-Pro-18A is a tandem dimer of 18A connected by a proline (Wool, G D et al. (2009) J. Lipid Res. 50:1889-1900). In some embodiments, a spin label is covalently attached to the mimetic at a single site in the mimetic. In some embodiments, the spin label is located in the center of the mimetic. ApoA-I mimetic 4F has the following amino acid sequence: Ac-DWFKAFYDKVAEKFKEAF-NH2 (SEQ ID NO:6) and 4F-Pro-4F is a tandem dimer of 4F connected by a proline residue (Wool, G D et al. (2009) J. Lipid Res. 50:1889-1900).

### Spin Labels

Spin labels are chemical compounds which are paramagnetic due to the presence of an unpaired electron in their structure. They are, therefore, a class of free radicals but are necessarily stable under conditions around normal temperature (below 100° C.) and physiological pH and also accommodate certain chemical reactions or experiments without affecting their free radical moiety. In some embodiments, the invention provides a spin-labeled lipoprotein probe with high specificity for HDL to measure the reverse cholesterol transport capacity of HDL in in vitro blood samples. In some embodiments, the spin label comprises an atom that bears a free electron. In some embodiments, the atom bearing a free electron is a nitrogen atom. In some embodiments, the spin label is a nitroxide. In some embodiments, the spin label is selected from (1-oxyl-2,2,5,5-tetramethyl-Δ3-pyrroline-3-methyl)methanethiosulfonate; (1-oxyl-2,2,5,5-tetramethyl-Δ3-pyrroline-3-methyl)methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl) methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl)methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; and 3-(2-iodo-acetamido-methyl)-PROXYL, free radical.

Spin-Labeled Lipoprotein Probe with High Specificity for HDL

In some embodiments, the invention provides a spin-labeled lipoprotein probe with high specificity for HDL to measure the reverse cholesterol transport capacity of HDL in blood. In some embodiments, the spin label is covalently attached to the lipoprotein. In some embodiments, the spin label is non-covalently attached to the lipoprotein. In some embodiments, the spin label associates with the lipoprotein. In some embodiments, the spin label is covalently attached to an amino acid residue on the lipoprotein. In some embodiments, the spin label is covalently attached to a cysteine residue on the lipoprotein. In some embodiments, the spin label is covalently attached to a cysteine residue on the lipoprotein through a thiosulfonate linkage.

In some embodiments of the invention, the spin label is covalently tethered to the lipoprotein by use of a spacer moiety between the spin label and the lipoprotein. Such a spacer can modulate the distance between the spin label and the lipoprotein and may impact the constraint of the spin label when attached to the lipoprotein. Examples of spacer moieties include alkanes such as methane, ethane, propane, butane and the like. In some embodiments, the spin label is covalently attached to a lipoprotein through a methylthiosulfonate linkage, an ethylthiosulfonate linkage, a propylthiosulfonate linkage, or a butylthiosulfonate linkage.

The invention provides methods to measure the capacity of HDL in an in vitro blood sample to support reverse cholesterol transport by means of EPR spectroscopy using a spin-labeled lipoprotein probe with high specificity for HDL. As the spin-labeled lipoprotein probe exchanges with lipoprotein in the HDL particle, it undergoes a conformation change. This change may be detected by a change in the EPR spectrum of the spin-label on the lipoprotein probe with high specificity for HDL. For example, a spin-labeled apoA-I lipoprotein probe will convert from a compact 4-helical bundle to an extended belt-like α-helix, which wraps around the perimeter of the nascent HDL particle upon lipidation. Spin-labeled lipoprotein probes may be designed to detect these structural changes upon binding to HDL. The site of the spin label on the spin-labeled lipoprotein probe is chosen based on the different spectra of the spin label when the lipoprotein is free/lipid-poor of lipid or bound to lipid. The spin label may be situated at any site on the lipoprotein. For example, lipoproteins may be genetically engineered to situate a unique cysteine residue at each position of the lipoprotein to create a library of lipoproteins for testing their utility as for the development of a spin-labeled lipoprotein probe. The spin label is then attached to the unique cysteine in each genetically engineered lipoprotein in the library. The library of candidate spin-labeled lipoprotein probes with high specificity for HDL are then tested by collecting the EPR spectra of the candidate probes bound to lipid or lipid-free/lipid-poor. Candidate spin-labeled lipoprotein probes which show detectable differences in the EPR spectra in lipid-bound versus lipid-free states are selected for use in the methods of the invention. In some embodiments of the invention, the spin-labeled lipoprotein is in the form of a dry powder; for example, a lyophilized preparation of the spin-labeled lipoprotein probe.

### Samples

Provided herein are for measuring the capacity of HDL to support reverse cholesterol transport in a sample by EPR spectroscopy of spin-labeled lipoprotein. In some embodiments, the sample is a biological sample. In some embodiments, the sample is a bodily fluid. In some embodiments the sample is a blood sample. In some embodiments, the sample is a cerebral spinal fluid. In yet other embodiments, the sample is a synthetically prepared sample used in drug discovery or health diagnostics development.

### Blood Samples

The invention methods for measuring the capacity of HDL to support reverse cholesterol transport in an in vitro blood sample by EPR spectroscopy of spin-labeled lipoprotein. In some embodiments of the invention, the in vitro blood sample is a whole blood sample. In some embodiments, the in vitro blood sample is a plasma sample. In some embodiments, the in vitro blood sample is a serum sample. In further embodiments, the in vitro blood sample comprises an anti-coagulant. In some embodiments, the anti-coagulant is heparin, coumadin, warfarin, EDTA, citrate or oxalate. In some embodiments, is collected from an individual into a vacucontainer. In some embodiments, the in vitro blood sample is analyzed by the methods of the invention following collection from the individual. In some embodiments, the in vitro blood sample frozen before analysis. In some embodiments, the in vitro blood sample undergoes one or two cycles of freezing and thawing prior to analysis.

### Methods of Measuring the Capacity of HDL to Support Reverse Cholesterol Transport

In some aspects, the invention provides methods for measuring the capacity of HDL to support reverse cholesterol transport in blood by adding a spin labeled lipoprotein probe with high specificity for HDL to an in vitro sample and collecting the electron paramagnetic resonance (EPR) spectrum of the sample (e.g. biological sample (e.g., blood, CSF, etc.) or synthetic sample). In some aspects, the invention provides methods for measuring the capacity of HDL to support reverse cholesterol transport in blood by adding a spin labeled lipoprotein probe with high specificity for HDL to an in vitro blood sample and collecting the electron paramagnetic resonance (EPR) spectrum of the sample. In some embodiments, the EPR spectrum of the sample is compared to EPR spectra for negative and/or positive controls. In some embodiments, the negative control is a lipid-free or lipid-poor spin-labeled lipoprotein probe. In some embodiments, the positive control is a spin-labeled lipoprotein probe bound to lipid; for example, dimyristoylphosphatidyl choline. In some embodiments, the EPR spectrum of the sample is the EPR spectrum of spin-labeled lipoprotein probe added to an in vitro blood sample from an individual with normal reverse cholesterol transport capacity; for example, from an individual not at risk for cardiovascular disease. In some embodiments, the EPR spectrum of a blood sample is the EPR spectrum of spin-labeled lipoprotein probe added to blood sample from an individual with normal reverse cholesterol transport capacity; for example, from an individual that is not diabetic. In some embodiments, the EPR spectrum of a CSF sample is the EPR spectrum of spin-labeled lipoprotein probe added to CSF sample from an individual with normal reverse cholesterol transport capacity; for example, from an individual not at risk for Alzheimer's disease.

In some embodiments of the invention, the reverse cholesterol transport capacity is a cholesterol efflux potential.

The spin-labeled lipoprotein probe comprises a lipoprotein with high specificity for HDL. A lipoprotein with high specificity for HDL is a lipoprotein where at least 60% of the lipoprotein associates with HDL when added to a sample (e.g, as described herein). A lipoprotein with high specificity for HDL is a lipoprotein where at least 60% of the lipoprotein associates with HDL when added to an in vitro blood sample. In some embodiments a lipoprotein with high specificity for HDL is a lipoprotein where at least 70% of the lipoprotein associates with HDL when added to a sample (e.g, as described herein). In some embodiments a lipoprotein with high specificity for HDL is a lipoprotein where at least 70% of the lipoprotein associates with HDL when added to an in vitro blood sample.

Methods of EPR spectroscopy are known in the art. General guidelines for performing EPR are provided by Klug, C S and Feix, J B (2008) Methods Cell Bio. 84:617-657), Fanucci G E and Cafiso, D S (2006) Curr. Opin. Struct. Bio. 16:644-653, and the EMX User's Manual. A nonlimiting exemplary method of EPR spectroscopy is based on Tetali, S D et al. (2010 J. Lipid Res. 51:1273-1283) as follows. EPR measurements are performed with a JEOL X-band spectrometer fitted with a loop-gap resonator. Spin-labeled lipoprotein probe with high specificity for HDL in TBS (10 mM Tris, pH 7.4, 150 mM NaCl and 0.005% sodium azide) is added to an in vitro blood sample. The sample is loaded into one-sided sealed glass capillaries and scanned by EPR. Vehicle controls are used. The spectra are obtained by an average of three scans (2 minutes each) over 100 G as a microwave power of 2 mW and a modulation amplitude of 1 G at room temperature or 37° C.

In some embodiments of the invention, the sample is scanned at 4° C. to establish a pre-exchange signal. The sample is then raised to 37° C. and scans are continued for 2 minutes, 4 minutes, 6 minutes, 10 minutes or more than 10 minutes.

In some embodiments of the invention, the spin-labeled lipoprotein probe is added to the sample (e.g., as described herein) at a concentration ranging from about 0.1 mg/ml to about 1.1 mg/ml. In some embodiments of the invention, the spin-labeled lipoprotein probe is added to the in vitro blood sample at a concentration ranging from about 0.1 mg/ml to about 1.1 mg/ml. In some embodiments of the invention, the spin-labeled lipoprotein probe is added to the sample (e.g., as described herein) at a concentration of about any of 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml or greater than 1.1 mg/ml. In some embodiments of the invention, the spin-labeled lipoprotein probe is added to the in vitro blood sample at a concentration of about any of 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml or greater than 1.1 mg/ml.

In some embodiments, the invention provides methods for measuring the capacity of HDL to support reverse cholesterol transport in a sample (e.g., as described herein (e.g, biological sample (e.g., blood, CSF, etc), synthetic sample) by EPR spectroscopy of spin-labeled lipoprotein with high specificity for HDL. In some embodiments, the invention provides methods for measuring the capacity of HDL to support reverse cholesterol transport in an in vitro blood sample by EPR spectroscopy of spin-labeled lipoprotein with high specificity for HDL. In some embodiments, the in vitro blood sample is a biological sample. In some embodiments, the in vitro blood sample is a whole blood sample. In some embodiments, the in vitro blood sample is a plasma sample. In some embodiments, the in vitro blood sample is a serum sample. In some embodiments, the in vitro sample is a CSF sample. In some embodiments, the sample is a synthetic sample. In further embodiments, the sample comprises an anti-coagulant. In further embodiments, the in vitro blood sample comprises an anti-coagulant. In some embodiments, the anti-coagulant is heparin, coumadin, warfarin, EDTA, citrate or oxalate. In some embodiments, the biological sample is collected from an individual into a vacucontainer. In some embodiments, the biological sample is analyzed by the methods of the invention following collection from the individual. In some embodiments, the in vitro blood sample is analyzed by the methods of the invention following collection from the individual. In some embodiments, the sample (e.g., as described herein) is frozen before analysis. In some embodiments, the sample (e.g., as described herein) undergoes one or two cycles of freezing and thawing prior to analysis. In some embodiments, the in vitro blood sample is frozen before analysis. In some embodiments, the in vitro blood sample undergoes one or two cycles of freezing and thawing prior to analysis.

In some embodiments of the invention, the EPR spectrum of the spin-labeled lipoprotein probe with high specificity for HDL is monitored over time following addition of the spin-labeled lipoprotein probe to the in vitro blood sample. In some embodiments, the EPR spectrum of the spin-labeled lipoprotein probe is monitored at one or more of the following times following addition of the spin-labeled lipoprotein probe to the in vitro blood sample: 1.0 min, 1.5 min, 2.0 min, 3.0 min, 4 min, 5 min, 6 min, 7 min, 8 min, 9 min, 10 min, 30 min, 60 min, or greater than 60 min.

In some embodiments of the invention, the amplitude of the center peak of the EPR spectrum is measured. The amplitude of the center peak is a measure of the distance between the baseline and the greatest signal from the baseline detected for the center peak. In some embodiments of the invention, the difference in the amplitude of the center peak of the EPR spectrum of the spin-labeled lipoprotein probe in the sample (e.g., as described herein) compared to the EPR spectrum of the negative control is indicative of a difference in the binding of the lipoprotein to the HDL. In some embodiments of the invention, the difference in the amplitude of the center peak of the EPR spectrum of the spin-labeled lipoprotein probe in the in vitro blood sample compared to the EPR spectrum of the negative control is indicative of a difference in the binding of the lipoprotein to the HDL. Depending on the location of the spin label on the spin-labeled lipoprotein probe, binding of the spin-labeled lipoprotein probe to HDL is indicated by an increase in amplitude of the center peak or a decrease in the amplitude of the center peak. Factors that influence the EPR spectrum of a spin label at a specific site on a lipoprotein include regional flexibility and solvent accessibility. In some embodiments, an increase in the amplitude of the center peak indicates an increase in the binding of the spin-labeled lipoprotein probe to the HDL. In other embodiments, an increase in the amplitude of the center peak indicates an decrease in the binding of the spin-labeled lipoprotein probe to the HDL. In yet other embodiments, a decrease in the amplitude of the center peak indicates an increase in the binding of the spin-labeled lipoprotein probe to the HDL. In some embodiments, a decrease in the amplitude of the center peak indicates an decrease in the binding of the spin-labeled lipoprotein probe to the HDL. In some embodiments, binding of a spin-labeled lipoprotein probe with high specificity for HDL, where the spin-labeled lipoprotein probe is an apoA-I protein with a nitroxide spin label covalently linked to cysteine residue situated at residue 219 of the apoA-I protein, is indicated by an increase in amplitude of the center peak of its EPR spectrum. In some embodiments, binding of a spin-labeled lipoprotein probe with high specificity for HDL, where the spin-labeled lipoprotein probe is an apoA-I protein with a nitroxide spin label covalently linked to cysteine residue situated at residue 111 of the apoA-I protein, is indicated by an increase in amplitude of the center peak of its EPR spectrum.

In some embodiments of the invention, the change in amplitude of the center peak is measured in relation to the amplitude of a near peak and/or a far peak of the EPR spectrum that does not change upon binding of the spin-labeled lipoprotein probe to HDL. As such, the amplitude of the near peak or far peak may serve as an internal control; for example, by indicating whether the spin label has been quenched.

In some embodiments, a change in the profile of the EPR spectrum is indicative of a change in the binding of the spin-labeled lipoprotein probe.

In some embodiments, a shift of the center with respect to the magnetic field strength is indicative of a change in the binding of the spin-labeled lipoprotein probe. In some embodiments, binding of the spin-labeled lipoprotein probe to HDL is indicated by increased magnetic field strength (shift to right along the X axis of the spectrum). In some embodiments, binding of the spin-labeled lipoprotein probe to HDL is indicated by decreased magnetic field strength (shift to left along the X axis of the spectrum).

The invention provides methods to measure the capacity of HDL to support reverse cholesterol transport by measuring the EPR spectrum of a spin-labeled lipoprotein probe with high specificity for HDL following addition of the spin-labeled lipoprotein probe to a sample (e.g, biological or synthetic samples as described herein. The invention provides methods to measure the capacity of HDL to support reverse cholesterol transport in blood by measuring the EPR spectrum of a spin-labeled lipoprotein probe with high specificity for HDL following addition of the spin-labeled lipoprotein probe to an in vitro blood sample. In some embodiments, binding of the spin-labeled lipoprotein probe to HDL is measured as the rate of binding. EPR spectra are collected over time and the change in the EPR spectra; for example, as measured by the change in amplitude of the center peak, is plotted against time. The slope of the curve of the plot is indicative of the rate of binding of the spin-labeled lipoprotein probe to the HDL. A fast rate of binding of the spin-labeled lipoprotein probe to the HDL reflects a high capacity of the HDL to support reverse cholesterol transport. A slow rate of binding of the spin-labeled lipoprotein probe to the HDL reflects a reduced capacity of the HDL to support reverse cholesterol transport or dysfunctional HDL. Rates of binding of the spin-labeled lipoprotein probes to HDL in blood may be compared with rates of binding of the spin-labeled lipoprotein probe to HDL in blood from individuals at low risk of cardiovascular disease or high risk of cardiovascular disease in order to assess the capacity of the HDL in a test blood sample to support reverse cholesterol transport.

The invention provides methods to measure the capacity of HDL to support reverse cholesterol transport by measuring the EPR spectrum of a spin-labeled lipoprotein probe with high specificity for HDL following addition of the spin-labeled lipoprotein probe to sample (e.g, biological or synthetic sample as described herein). The invention provides methods to measure the capacity of HDL to support reverse cholesterol transport in blood by measuring the EPR spectrum of a spin-labeled lipoprotein probe with high specificity for HDL following addition of the spin-labeled lipoprotein probe to an in vitro blood sample. In some embodiments, binding of the spin-labeled lipoprotein probe to HDL is measured as the time to equilibrium binding. EPR spectra are collected over time and the change in the EPR spectra; for example, as measured by the change in amplitude of the center peak, is plotted against time. The time to equilibrium binding is measured as the time to where the association rate of the spin-labeled lipoprotein to HDL is equal to the dissociation rate of binding of the spin-labeled lipoprotein to HDL. Time to equilibrium binding of the spin-labeled lipoprotein to HDL can be compared to a positive control or to the degree of binding of the spin-labeled lipoprotein to HDL in blood from one or more individuals with normal reverse cholesterol transport; for example, from individuals not at risk for cardiovascular disease. A time to equilibrium binding of about 5 min is indicative of a normal capacity of HDL to support reverse cholesterol transport in blood. A time to equilibrium binding of about 10 min or more is indicative of a reduced capacity of HDL to support reverse cholesterol transport in blood or dysfunctional HDL.

The invention provides methods to measure the capacity of HDL to support reverse cholesterol transport by measuring the EPR spectrum of a spin-labeled lipoprotein probe with high specificity for HDL following addition of the spin-labeled lipoprotein probe to a sample (e.g., biological or synthetic sample). The invention provides methods to measure the capacity of HDL to support reverse cholesterol transport in blood by measuring the EPR spectrum of a spin-labeled lipoprotein probe with high specificity for HDL following addition of the spin-labeled lipoprotein probe to an in vitro blood sample. In some embodiments, binding of the spin-labeled lipoprotein probe to HDL is measured as the degree of HDL binding. Such a measurement is an endpoint measurement. EPR spectra are collected over time and the change in the EPR spectra; for example, as measured by the change in amplitude of the center peak, is plotted against time. The degree of binding is measured as the equilibrium binding where the association rate of the spin-labeled lipoprotein to HDL is equal to the dissociation rate of binding of the spin-labeled lipoprotein to HDL. Degree of binding of the spin-labeled lipoprotein to HDL can be compared to a positive control or to the degree of binding of the spin-labeled lipoprotein to HDL in blood from one or more individuals with normal reverse cholesterol transport; for example, from individuals not at risk for cardiovascular disease. Degree of binding of the spin-labeled lipoprotein to HDL can be compared to a positive control or to the degree of binding of the spin-labeled lipoprotein to HDL in a sample from one or more individuals with normal reverse cholesterol transport; for example, from individuals not at risk for cardiovascular disease. In some embodiments, a degree of HDL binding of a spin-labeled lipoprotein probe with high specity for HDL from a test sample of 80% or less in indicative of reduced capacity of the HDL in the sample (e.g, biological or synthetic sample as described herein) for reverse cholesterol transport. In some embodiments, a degree of HDL binding of a spin-labeled lipoprotein probe with high specity for HDL from a test sample of 80% or less in indicative of reduced capacity of the HDL in the in vitro blood sample for reverse cholesterol transport. In some embodiments, a degree of HDL binding of a spin-labeled lipoprotein probe with high specity for HDL from a test sample of 80% or less in indicative of reduced capacity of the HDL in the in vitro blood sample for reverse cholesterol transport or dysfunctional HDL. In some embodiments, a degree of HDL binding of a spin-labeled lipoprotein probe with high specity for HDL from a test sample of 70% or less in indicative of reduced capacity of the HDL in the sample (e.g, biological or synthetic sample as described herein) for reverse cholesterol transport or dysfunctional HDL. In some embodiments, a degree of HDL binding of a spin-labeled lipoprotein probe with high specity for HDL from a test sample of 70% or less in indicative of reduced capacity of the HDL in the in vitro blood sample for reverse cholesterol transport or dysfunctional HDL. In some embodiments, a degree of HDL binding of a spin-labeled lipoprotein probe with high specity for HDL from a test sample of 60% or less in indicative of reduced capacity of the HDL in the sample (e.g, biological or synthetic sample as described herein) for reverse cholesterol transport or dysfunctional HDL. In some embodiments, a degree of HDL binding of a spin-labeled lipoprotein probe with high specity for HDL from a test sample of 60% or less in indicative of reduced capacity of the HDL in the in vitro blood sample for reverse cholesterol transport or dysfunctional HDL. In some embodiments, a degree of HDL binding of a spin-labeled lipoprotein probe with high specity for HDL from a test sample of 50% or less in indicative of reduced capacity of the HDL in the sample (e.g, biological or synthetic sample as described herein) for reverse cholesterol transport or dysfunctional HDL. In some embodiments, a degree of HDL binding of a spin-labeled lipoprotein probe with high specity for HDL from a test sample of 50% or less in indicative of reduced capacity of the HDL in the in vitro blood sample for reverse cholesterol transport or dysfunctional HDL.

The invention provides methods to measure the capacity of HDL to support reverse cholesterol transport by measuring the EPR spectrum of a spin-labeled lipoprotein probe with high specificity for HDL following addition of the spin-labeled lipoprotein probe to the sample (e.g, biological or synthetic sample as described herein). The invention provides methods to measure the capacity of HDL to support reverse cholesterol transport in blood by measuring the EPR spectrum of a spin-labeled lipoprotein probe with high specificity for HDL following addition of the spin-labeled lipoprotein probe to an in vitro blood sample. In some embodiments of the invention, the transition temperature of the HDL is determined. Binding of the spin-labeled lipoprotein probe with high specificity for HDL is added to samples at different temperatures; for example 0° C., 4° C., 10° C., 20° C., 25° C., 28° C., 30° C., 37° C. and EPR spectra are collected. Binding of the spin-labeled lipoprotein probe with high specificity for HDL is added to blood samples at different temperatures; for example 0° C., 4° C., 10° C., 20° C., 25° C., 28° C., 30° C., 37° C. and EPR spectra are collected. In some embodiments, a spin-labeled lipoprotein probe with high specificity for HDL is added to the sample (e.g, biological or synthetic sample as described herein). In some embodiments, a spin-labeled lipoprotein probe with high specificity for HDL is added to an in vitro blood sample. The EPA spectra of the same sample are collected at different temperatures by increasing or decreasing the temperature. For example, the EPR spectrum may be collected at 0° C., the temperature is then raised to 10° C. and the EPR spectrum is collected, the temperature is then raised to 20° C. and the EPR spectrum is collected, and the temperature is then raised to 37° C. and the EPR spectrum is collected. In some embodiments, the EPR spectra from a single sample are collected at 37° C., followed by collection at 20° C., followed by collection at 10° C., followed by collection at 0° C. Collection of EPR spectra at any combination of temperatures is contemplated. The transition temperature of the HDL is indicated by the lowest temperature at which the spin-labeled lipoprotein probe binds HDL as reflected by a change in the EPR spectrum. A transition temperature of about 25° C. or higher is indicative of HDL with reduced capacity of reverse cholesterol transport or dysfunctional HDL.

The invention provides methods to measure the capacity of HDL to support reverse cholesterol transport by measuring the EPR spectrum of a spin-labeled lipoprotein probe with high specificity for HDL following addition of the spin-labeled lipoprotein probe to the sample (e.g, biological or synthetic sample as described herein). The invention provides methods to measure the capacity of HDL to support reverse cholesterol transport in blood by measuring the EPR spectrum of a spin-labeled lipoprotein probe with high specificity for HDL following addition of the spin-labeled lipoprotein probe to an in vitro blood sample. In some embodiments, the biological sample is from a mammal. In some embodiments, the in vitro blood sample is from a mammal. In some embodiments, the biological sample is from a human, a mouse, a rat, a rabbit, a hamster, a guinea pig, a dog, a cat, or a pig. In some embodiments, the in vitro blood sample is from a human, a mouse, a rat, a rabbit, a hamster, a guinea pig, a dog, a cat, or a pig. In some embodiments the biological sample is a human biological sample as described herein. In some embodiments the sample is a human CSF sample. In some embodiments the blood sample is a human blood sample. In some embodiments, the biological sample is from a non-human mammal. In some embodiments, the blood sample is from a non-human mammal.

The invention provides methods to measure the capacity of HDL to support reverse cholesterol transport in blood by measuring the EPR spectrum of a spin-labeled lipoprotein probe with high specificity for HDL following addition of the spin-labeled lipoprotein probe to an in vitro blood sample. In some embodiments the blood sample is a human blood sample. In some embodiments, the blood sample is from a non-human mammal. In some embodiments, the blood sample is from an individual at risk for cardiovascular disease. In some embodiments the individual is a human. In some embodiments, the individual is a non-human mammal. In some embodiments the individual at risk for cardiovascular disease is diabetic. In some embodiments the individual is a human. In some embodiments, the individual is a non-human mammal. In some embodiments the individual at risk for cardiovascular disease is obese. In some embodiments the individual is a human. In some embodiments, the individual is a non-human mammal.

In some aspects, the invention provides methods of determining the risk for developing cardiovascular disease in an individual wherein the reverse cholesterol transport capacity of HDL in blood from the individual is measured by adding a spin-labeled lipoprotein probe with high specificity for HDL to an in vitro blood sample from the individual and the EPR spectrum of the spin-labeled lipoprotein probe is collected. The collected EPR spectrum is then compared to one or more negative controls and/or one or more positive controls. The negative control may be the EPR spectrum of a lipid-free or lipid-poor spin-labeled lipoprotein probe (e.g., where the ESR spectrum of the spin-labeled lipoprotein probe is not collected with the probe present in a blood sample; for example, the probe is present in e.g., a suitable buffer or other solvent system. The positive control may be a spin-labeled lipoprotein probe bound to lipid such as dimyristoylphosphatidyl choline (e.g., where the probe and lipid are present in e.g., a suitable buffer or other solvent system when the ESR spectrum is collected) or may be historical spectra of spin-labeled lipoprotein probes bound to HDL in blood samples from individuals not at risk for cardiovascular disease. A lower capacity of reverse cholesterol transport of the HDL in blood from the individual compared to positive controls indicates a risk for cardiovascular disease. In some embodiments a reverse cholesterol transport capacity of HDL of 80% normal indicates a risk for cardiovascular disease. In some embodiments a reverse cholesterol transport capacity of HDL of 70% normal indicates a risk for cardiovascular disease. In some embodiments a reverse cholesterol transport capacity of HDL of 60% normal indicates a risk for cardiovascular disease. In some embodiments a reverse cholesterol transport capacity of HDL of less than 50% normal indicates a risk for cardiovascular disease. In some embodiments, the individual is a human at risk for cardiovascular disease. In some embodiments the human at risk for cardiovascular disease is diabetic. In some embodiments the human at risk for cardiovascular disease is obese. In some embodiments, the cardiovascular disease is coronary artery disease. In some embodiments, the cardiovascular disease is atherosclerosis. In some embodiments, the cardiovascular disease is peripheral vascular disease. In some embodiments, the cardiovascular disease is stroke.

In some aspects, the invention provides methods of monitoring the course of therapy for cardiovascular disease in an individual wherein the reverse cholesterol transport capacity of HDL in blood from the individual is measured by adding a spin-labeled lipoprotein probe with high specificity for HDL to an in vitro blood sample from the individual and the EPR spectrum of the spin-labeled lipoprotein probe is collected, where the spin-labeled lipoprotein probe has high specificity for HDL. The reverse cholesterol transport capacity of HDL in blood from the individual undergoing therapy for cardiovascular disease is monitored over time during the course of the therapy. In some embodiments, the reverse cholesterol transport capacity of HDL in blood from the individual is measured prior to the onset of therapy. In some embodiments, the reverse cholesterol transport capacity of HDL in blood from the individual is measured before, during and/or after therapy. In some embodiments the individual is a human. In some embodiments the individual is a non-human mammal. In some embodiments, the cardiovascular disease is coronary artery disease, atherosclerosis, peripheral vascular disease or stroke. In some embodiments, an increase in the capacity of HDL to support reverse cholesterol transport indicates therapeutic efficacy. In some embodiments, a decrease in the capacity of HDL to support reverse cholesterol transport over time indicates a decrease in therapeutic efficacy. In some embodiments, a decrease in the capacity of HDL to support reverse cholesterol transport over time indicates a recurrence of the disease condition. In some embodiments, the reverse cholesterol transport capacity of HDL in blood from the individual undergoing therapy for cardiovascular disease is monitored by the methods of the invention over time following the course of the therapy to assess recurrence of the cardiovascular disease or risk of recurrence.

In some aspects, the invention provides methods of monitoring the course of therapy for Alzheimer's disease in an individual wherein the reverse cholesterol transport capacity of HDL in blood from the individual is measured by adding a spin-labeled lipoprotein probe with high specificity for HDL to a CSF sample from the individual and the EPR spectrum of the spin-labeled lipoprotein probe is collected, where the spin-labeled lipoprotein probe has high specificity for HDL. The reverse cholesterol transport capacity of HDL in blood from the individual undergoing therapy for Alzheimer's disease is monitored over time during the course of the therapy. In some embodiments, the reverse cholesterol transport capacity of HDL in CSF from the individual is measured prior to the onset of therapy. In some embodiments, the reverse cholesterol transport capacity of HDL in CSF from the individual is measured before, during and/or after therapy. In some embodiments the individual is a human. In some embodiments the individual is a non-human mammal. In some embodiments, an increase in the capacity of HDL to support reverse cholesterol transport indicates therapeutic efficacy. In some embodiments, a decrease in the capacity of HDL to support reverse cholesterol transport over time indicates a decrease in therapeutic efficacy. In some embodiments, a decrease in the capacity of HDL to support reverse cholesterol transport over time indicates a recurrence of the disease condition. In some embodiments, the reverse cholesterol transport capacity of HDL in CSF from the individual undergoing therapy for Alzheimer's disease is monitored by the methods of the invention over time following the course of the therapy to assess recurrence of the Alzheimer's disease or risk of recurrence.

In some aspects, the invention provides methods for evaluating known or potential therapeutics for cardiovascular disease, wherein the reverse cholesterol transport capacity of HDL in blood from an individual (e.g., a non-human test animal) is measured by adding a spin-labeled lipoprotein probe with high specificity for HDL to an in vitro blood sample from the individual and the EPR spectrum of the spin-labeled lipoprotein probe is collected, wherein the test animal has been subjected to the therapy. In some embodiments, the individual (e.g., a non-human test animal) has been subjected to the therapy by administration of the therapy. An increase in reverse cholesterol transport capacity is indicative of therapeutic efficacy. In some embodiments, the reverse cholesterol transport capacity of an in vitro blood sample from the individual (e.g., a non-human test animal) is determined one or more times during and/or after administering the therapy to the individual (e.g., a non-human test animal), wherein an increase in the reverse transport capacity of the in vitro blood sample from the test animal is indicative of therapeutic efficacy. In some embodiments, the non-human test animal is a mouse, a rat, a rabbit, a hamster, a guinea pig, a dog, a cat or a pig. In some embodiments, the individual is a human.

In some aspects, the invention provides methods for evaluating known or potential therapeutics for Alzheimer's disease, wherein the reverse cholesterol transport capacity of HDL in CSF from an individual (e.g., a non-human test animal) is measured by adding a spin-labeled lipoprotein probe with high specificity for HDL to an CSF sample from the individual and the EPR spectrum of the spin-labeled lipoprotein probe is collected, wherein the test animal has been subjected to the therapy. In some embodiments, the individual (e.g., a non-human test animal) has been subjected to the therapy by administration of the therapy. An increase in reverse cholesterol transport capacity is indicative of therapeutic efficacy. In some embodiments, the reverse cholesterol transport capacity of a CSF sample from the individual (e.g., a non-human test animal) is determined one or more times during and/or after administering the therapy to the individual (e.g., a non-human test animal), wherein an increase in the reverse transport capacity of the CSF sample from the test animal is indicative of therapeutic efficacy. In some embodiments, the non-human test animal is a mouse, a rat, a rabbit, a hamster, a guinea pig, a dog, a cat or a pig. In some embodiments, the individual is a human.

In some embodiments, the invention provides a method determining efficacy of a known or potential therapy for cardiovascular disease, wherein the reverse cholesterol transport capacity of HDL in blood from an individual (e.g., a non-human test animal) is measured by adding a spin-labeled lipoprotein probe with high specificity for HDL to an in vitro blood sample from the individual (e.g., a non-human test animal), administering the therapy to the individual (e.g., a non-human test animal), determining the reverse cholesterol transport capacity of the in vitro blood sample from the individual (e.g., a non-human test animal) one or more times during and/or after administering the therapy to the individual (e.g., a non-human test animal), wherein an increase in the reverse transport capacity of the in vitro blood sample from the individual (e.g., a non-human test animal) is indicative of therapeutic efficacy. In some embodiments, the non-human test animal is a mouse, a rat, a rabbit, a hamster, a guinea pig, a dog, a cat or a pig. In some embodiments the individual is a human.

In some embodiments, the invention provides a method determining efficacy of a known or potential therapy for Alzheimer's disease, wherein the reverse cholesterol transport capacity of HDL in CSF from an individual (e.g., a non-human test animal) is measured by adding a spin-labeled lipoprotein probe with high specificity for HDL to a CSF sample from the individual (e.g., a non-human test animal), administering the therapy to the individual (e.g., a non-human test animal), determining the reverse cholesterol transport capacity of the CSF sample from the individual (e.g., a non-human test animal) one or more times during and/or after administering the therapy to the individual (e.g., a non-human test animal), wherein an increase in the reverse transport capacity of the CSF sample from the individual (e.g., a non-human test animal) is indicative of therapeutic efficacy. In some embodiments, the non-human test animal is a mouse, a rat, a rabbit, a hamster, a guinea pig, a dog, a cat or a pig. In some embodiments the individual is a human.

### Kits

In some aspects, the invention provides kits for measuring the capacity of HDL to support reverse cholesterol transport by EPR, the kit comprising a spin-labeled lipoprotein probe with high specificity for HDL. In some aspects, the invention provides kits for measuring the capacity of HDL to support reverse cholesterol transport in blood by EPR, the kit comprising a spin-labeled lipoprotein probe with high specificity for HDL. In some embodiments, the reverse cholesterol transport is a cholesterol efflux potential. In some embodiments of the invention, the lipoprotein with high specificity for HDL is a lipoprotein where 60% or more, 70% or more, 80% or more or 90% or more of the lipoprotein associates with HDL. In some embodiments, a lipoprotein with high specificity for HDL is a lipoprotein where less than or about 40%, 30%, 20% or 10% associate with low density lipoproteins (VLD) or very low density lipoproteins (VLDL). In some embodiments, the HDL is HDL3. In some embodiments, the lipoprotein is not apoE4 or apoE2. In some embodiments, the lipoprotein is not apoE2. In some embodiments, the lipoprotein is not apo E4. In some embodiments, the lipoprotein is a human lipo protein. In some embodiments, the lipoprotein is a non-human mammalian lipoprotein.

In some embodiments, the invention provides kits for measuring the capacity of HDL to support reverse cholesterol transport in blood by EPR, the kit comprising a spin-labeled lipoprotein probe with high specificity for HDL. In some embodiments, the invention provides kits for determining the risk for developing cardiovascular disease in an individual by measuring the capacity of HDL to support reverse cholesterol transport in blood by EPR, the kit comprising a spin-labeled lipoprotein probe with high specificity for HDL. In some embodiments, the invention provides kits for assessing the course of therapy in an individual by measuring the capacity of HDL to support reverse cholesterol transport in blood by EPR, the kit comprising a spin-labeled lipoprotein probe with high specificity for HDL. In some embodiments, the individual is a human. In some embodiments, the invention provides kits for evaluating known or potential therapies for cardiovascular disease by measuring the capacity of HDL to support reverse cholesterol transport in blood of an individual (e.g., a non-human test animal) by EPR, the kit comprising a spin-labeled lipoprotein probe with high specificity for HDL. In some embodiments, the kit further comprises one or more anti-coagulants and/or a vacutainer for collection of the blood sample. In some embodiments, the apoE protein is not an apoE4 protein.

In some embodiments, the invention provides kits for measuring the capacity of HDL to support reverse cholesterol transport in CSF by EPR, the kit comprising a spin-labeled lipoprotein probe with high specificity for HDL. In some embodiments, the invention provides kits for determining the risk for developing or having Alzheimer's disease in an individual by measuring the capacity of HDL to support reverse cholesterol transport in blood by EPR, the kit comprising a spin-labeled lipoprotein probe with high specificity for HDL. In some embodiments, the invention provides kits for assessing the course of therapy in an individual by measuring the capacity of HDL to support reverse cholesterol transport in CSF by EPR, the kit comprising a spin-labeled lipoprotein probe with high specificity for HDL. In some embodiments, the individual is a human. In some embodiments, the invention provides kits for evaluating known or potential therapies for Alzheimer's disease by measuring the capacity of HDL to support reverse cholesterol transport in blood of an individual (e.g., a non-human test animal) by EPR, the kit comprising a spin-labeled lipoprotein probe with high specificity for HDL.

In some aspects, the invention provides kits for measuring the capacity of HDL to support reverse cholesterol transport by EPR, the kit comprising a spin-labeled lipoprotein probe with high specificity for HDL wherein the lipoprotein is an apoA-I or fragment thereof. In some aspects, the invention provides kits for measuring the capacity of HDL to support reverse cholesterol transport in blood by EPR, the kit comprising a spin-labeled lipoprotein probe with high specificity for HDL wherein the lipoprotein is an apoA-I or fragment thereof. In some embodiments, the apoA-I protein is a human apoA-I protein. In some embodiments, the has the sequence of SEQ ID NO:1 or a fragment thereof. In some embodiments, the spin label is located at a single residue on the apoA-I protein or fragment thereof. In some embodiments, the apoA-I probe comprises two spin-labels, each at a single amino acid residue in the apoA-I protein. In some embodiments, the spin label is covalently attached to the apoA-I protein or fragment thereof. In some embodiments, the spin label is non-covalently attached or associated with the apoA-I protein or fragment thereof. In some embodiments the spin label is attached to a cysteine residue in the apoA-I protein. The native apoA-I protein does not contain a cysteine residue. In some embodiments of the invention, the apoA-I is engineered to contain a cysteine residue by replacing a native amino acid residue with a cysteine residue. This provides a means for specifically directing the spin label to a single site on the apoA-I protein with a reduced risk of generating a spin-labeled apoA-I protein in which a portion of the spin-labels are attached to the apoA-I protein in a random fashion. In some embodiments of the invention, the apoA-I protein is engineered to locate single cysteine residue at any site from residue 188 to residue 243. In some embodiments, the spin label is attached to the single cysteine residue genetically engineered at any site from residue 188 to residue 243. In some embodiments, the spin label is attached to a residue of apoA-I at any site from residue 188 to residue 243. In some embodiments of the invention, the spin label is attached to a cysteine genetically engineered to sites 98, 111 or 217 of the apoA-I protein. In some embodiments of the invention, the spin label is attached to a residue of the apoA-I protein to sites 98, 111 or 217 of the apoA-I protein. In some embodiments, the spin label is covalently attached to a cysteine residue at position 217 of the apoA-I protein. In some embodiments, the spin label is covalently attached to an amino acid at position 26, 44, 64, 101, 167, or 226 of the apoA-I lipoprotein. In some embodiments, the native amino acid residue at position 26, 44, 64, 101, 167, or 226 has been replaced by a cysteine residue. In some embodiments, the spin label is attached to residue 217 of the apoA-I protein. In some embodiments, the spin label is attached to a cysteine residue genetically engineered to site 217 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 217 of the apoA-I protein (SEQ ID NO:2). In some embodiments, the spin label is attached to residue 26 of the apoA-I protein. In some embodiments, the spin label is attached to a cysteine residue genetically engineered to site 26 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 26 of the apoA-I protein (SEQ ID NO:2). In some embodiments, the spin label is attached to residue 44 of the apoA-I protein. In some embodiments, the spin label is attached to a cysteine residue genetically engineered to site 44 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 44 of the apoA-I protein (SEQ ID NO:2). In some embodiments, the spin label is attached to residue 64 of the apoA-I protein. In some embodiments, the spin label is attached to a cysteine residue genetically engineered to site 64 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 64 of the apoA-I protein (SEQ ID NO:2). In some embodiments, the spin label is attached to residue 101 of the apoA-I protein. In some embodiments, the spin label is attached to a cysteine residue genetically engineered to site 101 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 101 of the apoA-I protein (SEQ ID NO:2). In some embodiments, the spin label is attached to residue 111 of the apoA-I protein. In some embodiments, the spin label is attached to a cysteine residue genetically engineered to site 111 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 111 of the apoA-I protein (SEQ ID NO:2). In some embodiments, the spin label is attached to residue 98 of the apoA-I protein. In some embodiments, the spin label is attached to a cysteine residue genetically engineered to site 98 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 98 of the apoA-I protein (SEQ ID NO:2). In some embodiments, the spin label is attached to residue 167 of the apoA-I protein. In some embodiments, the spin label is attached to a cysteine residue genetically engineered to site 167 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 167 of the apoA-I protein (SEQ ID NO:2). In some embodiments, the spin label is attached to residue 226 of the apoA-I protein. In some embodiments, the spin label is attached to a cysteine residue genetically engineered to site 226 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 226 of the apoA-I protein (SEQ ID NO:2). In some embodiments, the apoA-I protein is a non-human mammalian apoA-I protein. In some embodiments, the kit further comprises one or more anti-coagulants and/or a vacutainer for collection of the sample (e.g. a biological or synthetic sample as described herein). In some embodiments, the kit further comprises one or more anti-coagulants and/or a vacutainer for collection of the blood sample.

In some embodiments, the kit comprises a spin-labeled apoA-II protein or fragment thereof with high specificity for HDL. In some embodiments, the apoA-II protein is a human apoA-II protein. In some embodiments, the spin label is covalently attached to the apoA-II protein or fragment thereof. In some embodiments, the spin label is non-covalently attached or associated with the apoA-II protein or fragment thereof. In some embodiments of the invention, the apoA-II protein or fragment thereof comprises two spin-labels, each at a single amino acid residue in the apoA-II protein. In some embodiments the spin label is attached to a cysteine residue in the apoA-II protein or fragment thereof. The native apoA-II protein contains one cysteine residue located in the signal peptide. The mature apoA-II protein does not contain a cysteine residue. In some embodiments of the invention, the mature apoA-II protein is engineered to locate single cysteine residue at any site from residue 24 to residue 100. In some embodiments of the invention, the apoA-II precursor is engineered to replace the native cysteine residue in the signal peptide with another amino acid residue and engineered to contain another cysteine residue by replacing a native amino acid residue with a cysteine residue. In some embodiments, the spin label is attached to the engineered cysteine residue of the apoA-II protein. In some embodiments, the apoA-II protein is a non-human mammalian apoA-II protein. In some embodiments, the kit further comprises one or more anti-coagulants and/or a vacutainer for collection of the blood sample.

In some embodiments, the kit comprises a spin-labeled apoE protein or fragment thereof with high specificity for HDL. In some embodiments, the apoE protein is a human apoE protein. In some embodiments, the apoE protein is an apoE3 protein. In some embodiments, the apoE protein is not an apoE4 protein. In some embodiments the apoE protein is not an apoE2 protein. In some embodiments, the spin label is located at a single residue on the apoE protein or fragment thereof. In some embodiments, the spin label is covalently attached to the apoE protein or fragment thereof. In some embodiments, the spin label is non-covalently attached or associated with the apoE protein or fragment thereof. In some embodiments of the invention, the apoE protein or fragment thereof comprises two spin-labels, each at a single amino acid residue in the apoE protein. In some embodiments the spin label is attached to a cysteine residue in the apoE protein or fragment thereof. The native apoE protein contains two cysteine residues, one located in the signal peptide and one located in the mature apoE protein. In some embodiments of the invention, the apoE protein is engineered to replace the native cysteine residues and engineered to contain another cysteine residue by replacing a native amino acid residue with a cysteine residue.

In some embodiments, the invention provides kits comprising a spin-labeled lipoprotein probe with high specificity for HDL, wherein the spin-labeled lipoprotein comprises a mimetic of a lipoprotein. In some embodiments, the mimetic of a lipoprotein is a mimetic of apoA-I. In some embodiments the apoA-I mimetic is a mimetic of a non-human mammalian apoA-I protein. In some embodiments the apoA-I mimetic is a mimetic of human apoA-I protein. In some embodiments, the apoA-I mimetic is 18A, 18A-Pro-18A, 4F and 4f-Pro-4F. In some embodiments, a spin label is covalently attached to the mimetic at a single site in the mimetic. In some embodiments, the spin label is located in the center of the mimetic. ApoA-I mimetic 4F has the following amino acid sequence: Ac-DWFKAFYDKVAEKFKEAF-NH2 (SEQ ID NO:6) and 4F-Pro-4F is a tandem dimer of 4F connected by a proline residue (Wool, G D et al. (2009) J. Lipid Res. 50:1889-1900). In some embodiments, the apoE protein is a non-human mammalian apoE protein. In some embodiments, the kit further comprises one or more anti-coagulants and/or a vacutainer for collection of the blood sample.

In some aspects, the invention provides kits for measuring the capacity of HDL to support reverse cholesterol transport by EPR, the kit comprising a spin-labeled lipoprotein probe with high specificity for HDL. In some aspects, the invention provides kits for measuring the capacity of HDL to support reverse cholesterol transport in blood by EPR, the kit comprising a spin-labeled lipoprotein probe with high specificity for HDL. In some embodiments, the spin label comprises an atom that bears a free electron. In some embodiments, the atom bearing a free electron is a nitrogen atom. In some embodiments, the spin label is a nitroxide. In some embodiments, the spin label is selected from (1-oxyl-2,2,5,5-tetramethyl-Δ3-pyrroline-3-methyl) methanethiosulfonate; (1-oxyl-2,2,5,5-tetramethyl-Δ3-pyrroline-3-methyl) methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; and 3-(2-iodo-acetamido-methyl)-PROXYL, free radical. In some embodiments, the spin-label is a perdeuterated spin label. In some embodiments, the spin label is not (1-oxyl-2,2,5,5-tetramethyl-Δ3-pyrroline-3-methyl)methanethiosulfonate.

In some aspects, the invention provides kits for measuring the capacity of HDL to support reverse cholesterol transport by EPR, the kit comprising a spin-labeled lipoprotein probe with high specificity for HDL. In some aspects, the invention provides kits for measuring the capacity of HDL to support reverse cholesterol transport in blood by EPR, the kit comprising a spin-labeled lipoprotein probe with high specificity for HDL. In some embodiments, the kit comprises a spin-labeled lipoprotein probe with high specificity for HDL to measure the reverse cholesterol transport capacity of HDL. In some embodiments, the kit comprises a spin-labeled lipoprotein probe with high specificity for HDL to measure the reverse cholesterol transport capacity of HDL in blood. In some embodiments, the spin label is covalently attached to the lipoprotein. In some embodiments, the spin label is non-covalently attached to the lipoprotein. In some embodiments, the spin label associates with the lipoprotein. In some embodiments, the spin label is covalently attached to an amino acid residue on the lipoprotein. In some embodiments, the spin label is covalently attached to a cysteine residue on the lipoprotein. In some embodiments, the spin label is covalently attached to a cysteine residue on the lipoprotein through a thiosulfonate linkage. In some embodiments, the kit further comprises one or more anti-coagulants and/or a vacutainer for collection of the blood sample.

In some aspects, the invention provides kits for measuring the capacity of HDL to support reverse cholesterol transport by EPR, the kit comprising a spin-labeled lipoprotein probe with high specificity for HDL. In some aspects, the invention provides kits for measuring the capacity of HDL to support reverse cholesterol transport in blood by EPR, the kit comprising a spin-labeled lipoprotein probe with high specificity for HDL. In some embodiments of the invention, the spin label is covalently tethered to the lipoprotein by use of a spacer moiety between the spin label and the lipoprotein. Such a spacer can modulate the distance between the spin label and the lipoprotein and may impact the constraint of the spin label when attached to the lipoprotein. Examples of spacer moieties include alkanes such as methane, ethane, propane, butane and the like. In some embodiments, the spin label is covalently attached to a lipoprotein through a methylthiosulfonate linkage, an ethylthiosulfonate linkage, a propylthiosulfonate linkage, or a butylthiosulfonate linkage. In some embodiments, the kit further comprises one or more anti-coagulants and/or a vacutainer for collection of the blood sample.

In some embodiments, the kit is formulated to provide a spin-labeled lipoprotein probe with high specificity for HDL at a concentration of about 0.1 mg/ml to about 1.1 mg/ml. In some embodiments, the kit is formulated to provide a spin-labeled lipoprotein probe with high specificity for HDL at a concentration of about 0.3 mg/ml. In some embodiments the kit is formulated to provide a spin-labeled lipoprotein probe with high specificity for HDL at a concentration of greater than about 0.8 mg/ml. In some embodiments, the kit further comprises one or more anti-coagulants and/or a vacutainer for collection of the sample (e.g., biological or synthetic samples described herein). In some embodiments, the kit further comprises one or more anti-coagulants and/or a vacutainer for collection of the blood sample.

In some aspects, the invention provides kits for measuring the capacity of HDL to support reverse cholesterol transport by EPR, the kit comprising a spin-labeled lipoprotein probe with high specificity for HDL. In some aspects, the invention provides kits for measuring the capacity of HDL to support reverse cholesterol transport in blood by EPR, the kit comprising a spin-labeled lipoprotein probe with high specificity for HDL. The spin-labeled lipoprotein of the kit is formulated for use in methods to measure the capacity of HDL to support reverse cholesterol transport by EPR spectroscopy of spin-labeled lipoprotein. In some embodiments, the spin-labeled lipoprotein of the kit is formulated for use in methods to measure the capacity of HDL to support reverse cholesterol transport in an in vitro blood sample by EPR spectroscopy of spin-labeled lipoprotein. In some embodiments of the invention, the sample is a biological sample. In some embodiments of the invention, the sample is a synthetic sample. In some embodiments of the invention, the in vitro blood sample is a whole blood sample. In some embodiments, the in vitro blood sample is a plasma sample. In some embodiments, the in vitro blood sample is a serum sample. In some embodiments, the sample is a CSF sample. In some embodiments, the biological sample is from a mammal such as a human, a mouse, a rat, a rabbit, a hamster, a guinea pig or a pig. In some embodiments, the in vitro blood sample is from a mammal such as a human, a mouse, a rat, a rabbit, a hamster, a guinea pig or a pig. In some embodiments, the biological sample is from a non-human mammal. In some embodiments, the biological sample is from a human. In some embodiments, the kit further comprises an anti-coagulant. In some embodiments, the anti-coagulant is heparin, coumadin, warfarin, EDTA, citrate or oxalate. In some embodiments, the biological sample is collected from an individual into a vacucontainer. In some embodiments, the blood sample is collected from an individual into a vacucontainer. In some embodiments, the kits further comprise buffers, syringes and the like suitable for EPR analysis of samples (e.g., biological or synthetic samples as described herein). In some embodiments, the kits further comprise buffers, syringes and the like suitable for EPR analysis of blood samples.

Suitable packaging for compositions described herein are known in the art, and include, for example, vials (e.g., sealed vials), vessels, ampules, bottles, jars, flexible packaging (e.g., sealed Mylar or plastic bags), and the like. Packaging for compositions may also include capillary tubes or flatcell tubes. These articles of manufacture may further be sterilized and/or sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of spin-labeled lipoproteins with high affinity for HDL generally include information as to use.

In some embodiments, spin-labeled lipoprotein as described herein may be lyophilized and provided in a capillary tube or a flatcell tube (e.g., glass capillary tube or others known in the art). The capillary tube or flatcell tube may also optionally include an EPR reference standard as described herein.

In some embodiments, the tube is made of a non-paramagnetic material. In some embodiments, the tube comprises glass, plastic, polymer or quartz. The interior of the tube can be any dimension. In some embodiments, the interior of the tube is round. In some embodiments, the interior of the tube is rectangular. In some embodiments of the invention, the interior of the tube is flat rectangular. In some embodiments, the tube is a single-bore tube. In some embodiments, the tube is a multi-bore tube.

### Compositions

In some aspects, the invention provides compositions comprising an in vitro blood sample and a spin-labeled lipoprotein with high specificity for HDL. In some embodiments of the invention, the lipoprotein with high specificity for HDL is a lipoprotein where 60% or more, 70% or more, 80% or more or 90% or more of the lipoprotein associates with HDL. In some embodiments, a lipoprotein with high specificity for HDL is a lipoprotein where less than or about 40%, 30%, 20% or 10% associate with low density lipoproteins (VLD) or very low density lipoproteins (VLDL). In some embodiments, the HDL is HDL3. In some embodiments, the lipoprotein is not apoE4 or apoE2. In some embodiments, the lipoprotein is not apoE2. In some embodiments, the lipoprotein is not apo E4. In some embodiments, the lipoprotein is a human lipo protein. In some embodiments, the lipoprotein is a non-human mammalian lipoprotein.

In some aspects, the invention provides compositions comprising an in vitro blood sample and a spin-labeled lipoprotein probe with high specificity for HDL wherein the lipoprotein is an apoA-I or fragment thereof. In some embodiments, the apoA-I protein is a human apoA-I protein. In some embodiments, the apoA-I has the sequence of SEQ ID NO:1 or a fragment thereof. In some embodiments, the spin label is located at a single residue on the apoA-I protein or fragment thereof. In some embodiments, the apoA-I probe comprises two spin-labels, each at a single amino acid residue in the apoA-I protein. In some embodiments, the spin label is covalently attached to the apoA-I protein or fragment thereof. In some embodiments, the spin label is non-covalently attached or associated with the apoA-I protein or fragment thereof. In some embodiments the spin label is attached to a cysteine residue in the apoA-I protein. The native apoA-I protein does not contain a cysteine residue. In some embodiments of the invention, the apoA-I is engineered to contain a cysteine residue by replacing a native amino acid residue with a cysteine residue. This provides a means for specifically directing the spin label to a single site on the apoA-I protein with a reduced risk of generating a spin-labeled apoA-I protein in which a portion of the spin-labels are attached to the apoA-I protein in a random fashion. In some embodiments of the invention, the apoA-I protein is engineered to locate single cysteine residue at any site from residue 188 to residue 243. In some embodiments, the spin label is attached to the single cysteine residue genetically engineered at any site from residue 188 to residue 243. In some embodiments, the spin label is attached to a residue of apoA-I at any site from residue 188 to residue 243. In some embodiments of the invention, the spin label is attached to a cysteine genetically engineered to sites 98, 111 or 217 of the apoA-I protein. In some embodiments of the invention, the spin label is attached to a residue of the apoA-I protein to sites 98, 111 or 217 of the apoA-I protein. In some embodiments, the spin label is covalently attached to a cysteine residue at position 217 of the apoA-I protein. In some embodiments, the spin label is covalently attached to an amino acid at position 26, 44, 64, 101, 167, or 226 of the apoA-I lipoprotein. In some embodiments, the native amino acid residue at position 26, 44, 64, 101, 167, or 226 has been replaced by a cysteine residue. In some embodiments, the spin label is attached to residue 217 of the apoA-I protein. In some embodiments, the spin label is attached to a cysteine residue genetically engineered to site 217 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 217 of the apoA-I protein (SEQ ID NO:2). In some embodiments, the spin label is attached to residue 26 of the apoA-I protein. In some embodiments, the spin label is attached to a cysteine residue genetically engineered to site 26 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 26 of the apoA-I protein (SEQ ID NO:2). In some embodiments, the spin label is attached to residue 44 of the apoA-I protein. In some embodiments, the spin label is attached to a cysteine residue genetically engineered to site 44 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 44 of the apoA-I protein (SEQ ID NO:2). In some embodiments, the spin label is attached to residue 64 of the apoA-I protein. In some embodiments, the spin label is attached to a cysteine residue genetically engineered to site 64 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 64 of the apoA-I protein (SEQ ID NO:2). In some embodiments, the spin label is attached to residue 98 of the apoA-I protein. In some embodiments, the spin label is attached to a cysteine residue genetically engineered to site 98 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 98 of the apoA-I protein (SEQ ID NO:2). In some embodiments, the spin label is attached to residue 101 of the apoA-I protein. In some embodiments, the spin label is attached to a cysteine residue genetically engineered to site 101 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 101 of the apoA-I protein (SEQ ID NO:2). In some embodiments, the spin label is attached to residue 111 of the apoA-I protein. In some embodiments, the spin label is attached to a cysteine residue genetically engineered to site 111 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 111 of the apoA-I protein (SEQ ID NO:2). In some embodiments, the spin label is attached to residue 167 of the apoA-I protein. In some embodiments, the spin label is attached to a cysteine residue genetically engineered to site 167 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 167 of the apoA-I protein (SEQ ID NO:2). In some embodiments, the spin label is attached to residue 226 of the apoA-I protein. In some embodiments, the spin label is attached to a cysteine residue genetically engineered to site 226 of the apoA-I protein (SEQ ID NO:2). In some embodiments of the invention, the spin label is a (1-oxyl-2,2,5,5-tetramethyl-Δ3-pyrroline-3-methyl)methanethiosulfonate or (1-oxyl-2,2,5,5-tetramethylpyrrolidin-3-yl)methyl methanesulfonate and is covalently attached to a cysteine residue genetically engineered to position 226 of the apoA-I protein (SEQ ID NO:2). In some embodiments, the apoA-I is a non-human apoA-I.

In some embodiments, the invention provides a composition comprising a sample (e.g., a biological sample or synthetic sample as described herein) and a spin-labeled apoA-II protein or fragment thereof with high specificity for HDL. In some embodiments, the invention provides a composition comprising an in vitro blood sample and a spin-labeled apoA-II protein or fragment thereof with high specificity for HDL. In some embodiments, the apoA-II protein is a human apoA-II protein. In some embodiments, the spin label is covalently attached to the apoA-II protein or fragment thereof. In some embodiments, the spin label is non-covalently attached or associated with the apoA-II protein or fragment thereof. In some embodiments of the invention, the apoA-II protein or fragment thereof comprises two spin-labels, each at a single amino acid residue in the apoA-II protein. In some embodiments the spin label is attached to a cysteine residue in the apoA-II protein or fragment thereof. The native apoA-II protein contains one cysteine residue located in the signal peptide. The mature apoA-II protein does not contain a cysteine residue. In some embodiments of the invention, the mature apoA-II protein is engineered to locate single cysteine residue at any site from residue 24 to residue 100. In some embodiments of the invention, the apoA-II precursor is engineered to replace the native cysteine residue in the signal peptide with another amino acid residue and engineered to contain another cysteine residue by replacing a native amino acid residue with a cysteine residue. In some embodiments, the spin label is attached to the engineered cysteine residue of the apoA-II protein. In some embodiments, the apoA-II protein is a non-human apoA-II protein.

In some embodiments, the invention provides a composition comprising a sample (e.g., a biological sample or synthetic sample as described herein) and a spin-labeled apoE protein or fragment thereof with high specificity for HDL In some embodiments, the invention provides a composition comprising an in vitro blood sample and a spin-labeled apoE protein or fragment thereof with high specificity for HDL. In some embodiments, the apoE protein is a human apoE protein. In some embodiments, the apoE protein is an apoE3 protein. In some embodiments, the apoE protein is not an apoE4 protein. In some embodiments, the apoE protein is not an apoE2 protein. In some embodiments, the spin label is located at a single residue on the apoE protein or fragment thereof. In some embodiments, the spin label is covalently attached to the apoE protein or fragment thereof. In some embodiments, the spin label is non-covalently attached or associated with the apoE protein or fragment thereof. In some embodiments of the invention, the apoE protein or fragment thereof comprises two spin-labels, each at a single amino acid residue in the apoE protein. In some embodiments the spin label is attached to a cysteine residue in the apoE protein or fragment thereof. The native apoE protein contains two cysteine residues, one located in the signal peptide and one located in the mature apoE protein. In some embodiments of the invention, the apoE protein is engineered to replace the native cysteine residues and engineered to contain another cysteine residue by replacing a native amino acid residue with a cysteine residue. In some embodiments, the apoE protein is a non-human apoE protein.

In some embodiments, the invention provides compositions comprising a sample (e.g., a biological sample or synthetic sample as described herein) and a spin-labeled lipoprotein probe with high specificity for HDL, wherein the spin-labeled lipoprotein comprises a mimetic of a lipoprotein. In some embodiments, the invention provides compositions comprising an in vitro blood sample and a spin-labeled lipoprotein probe with high specificity for HDL, wherein the spin-labeled lipoprotein comprises a mimetic of a lipoprotein. In some embodiments, the mimetic of a lipoprotein is a mimetic of apoA-I. In some embodiments the apoA-I mimetic is a mimetic of a non-human mammalian apoA-I protein. In some embodiments the apoA-I mimetic is a mimetic of human apoA-I protein. In some embodiments, the apoA-I mimetic is 18A, 18A-Pro-18A, 4F and 4f-Pro-4F. In some embodiments, a spin label is covalently attached to the mimetic at a single site in the mimetic. In some embodiments, the spin label is located in the center of the mimetic. ApoA-I mimetic 4F has the following amino acid sequence: Ac-DWFKAFYDKVAEKFKEAF-NH2 (SEQ ID NO:6) and 4F-Pro-4F is a tandem dimer of 4F connected by a proline residue (Wool, G D et al. (2009) J. Lipid Res. 50:1889-1900).

In some aspects, the invention provides a composition comprising a sample (e.g., a biological sample or synthetic sample as described herein) and a spin-labeled lipoprotein probe with high specificity for HDL. In some aspects, the invention provides a composition comprising an in vitro blood sample and a spin-labeled lipoprotein probe with high specificity for HDL. In some embodiments, the spin label comprises an atom that bears a free electron. In some embodiments, the atom bearing a free electron is a nitrogen atom. In some embodiments, the spin label is a nitroxide. In some embodiments, the spin label is selected from (1-oxyl-2,2,5,5-tetramethyl-Δ3-pyrroline-3-methyl)methanethiosulfonate; (1-oxyl-2,2,5,5-tetramethyl-Δ3-pyrroline-3-methyl)methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; and 3-(2-iodo-acetamido-methyl)-PROXYL, free radical.

In some aspects, the invention provides compositions comprising a sample (e.g., a biological sample or synthetic sample as described herein) and a spin-labeled lipoprotein probe with high specificity for HDL. In some aspects, the invention provides compositions comprising an in vitro blood sample and a spin-labeled lipoprotein probe with high specificity for HDL. In some embodiments, the spin label is covalently attached to the lipoprotein. In some embodiments, the spin label is non-covalently attached to the lipoprotein. In some embodiments, the spin label associates with the lipoprotein. In some embodiments, the spin label is covalently attached to an amino acid residue on the lipoprotein. In some embodiments, the spin label is covalently attached to a cysteine residue on the lipoprotein. In some embodiments, the spin label is covalently attached to a cysteine residue on the lipoprotein through a thiosulfonate linkage. In some embodiments the lipoprotein is a human lipoprotein. In some embodiments the lipoprotein is a non-human mammalian protein.

In some embodiments, the invention provides compositions comprising a sample (e.g., a biological sample or synthetic sample as described herein) and a spin-labeled lipoprotein probe with high specificity for HDL. In some embodiments, the invention provides compositions comprising an in vitro blood sample and a spin-labeled lipoprotein probe with high specificity for HDL. In some embodiments, the spin label is covalently tethered to the lipoprotein by use of a spacer moiety between the spin label and the lipoprotein. Examples of spacer moieties include alkanes such as methane, ethane, propane, butane and the like. In some embodiments, the spin label is covalently attached to a lipoprotein through a methylthiosulfonate linkage, an ethylthiosulfonate linkage, a propylthiosulfonate linkage, or a butylthiosulfonate linkage. In some embodiments the lipoprotein is a human lipoprotein. In some embodiments the lipoprotein is a non-human mammalian protein.

In some embodiments, the invention provides compositions comprising a sample (e.g., a biological sample or synthetic sample as described herein) and a spin-labeled lipoprotein probe with high specificity for HDL. In some embodiments, the invention provides compositions comprising an in vitro blood sample and a spin-labeled lipoprotein probe with high specificity for HDL. In some embodiments the sample is a biological sample. In some embodiments the sample is a synthetic sample. In some embodiments, the in vitro blood sample is a whole blood sample. In some embodiments, the in vitro blood sample is a plasma sample. In some embodiments, the in vitro blood sample is a serum sample. In some embodiments the sample is a CSF sample. In some embodiments, the biological sample is from a mammal such as a human, a mouse, a rat, a rabbit, a hamster, a guinea pig or a pig. In some embodiments the mammal is a human. In some embodiments the mammal is a non-human animal (e.g., a mouse, a rat, a rabbit, a hamster, a guinea pig, a pig, etc.). In some embodiments, the in vitro blood sample is from a mammal such as a human, a mouse, a rat, a rabbit, a hamster, a guinea pig or a pig. In some embodiments the mammal is a human. In some embodiments the mammal is a non-human animal (e.g., a mouse, a rat, a rabbit, a hamster, a guinea pig, a pig, etc.). In some embodiments, the composition further comprises an anti-coagulant. In some embodiments, the anti-coagulant is heparin, coumadin, warfarin, EDTA, citrate or oxalate.

In some aspects, the invention provides a composition comprising a spin-labeled lipoprotein probe with high specificity for HDL wherein the lipoprotein is an apoA-II protein or fragment thereof. In some embodiments, the apoA-II protein is a human apoA-II protein. In some embodiments, the spin label is covalently attached to the apoA-II protein or fragment thereof. In some embodiments, the spin label is non-covalently attached or associated with the apoA-II protein or fragment thereof. In some embodiments of the invention, the apoA-II protein or fragment thereof comprises two spin-labels, each at a single amino acid residue in the apoA-II protein. In some embodiments the spin label is attached to a cysteine residue in the apoA-II protein or fragment thereof. The native apoA-II protein contains one cysteine residue located in the signal peptide. The mature apoA-II protein does not contain a cysteine residue. In some embodiments of the invention, the mature apoA-II protein is engineered to locate single cysteine residue at any site from residue 24 to residue 100. In some embodiments of the invention, the apoA-II precursor is engineered to replace the native cysteine residue in the signal peptide with another amino acid residue and engineered to contain another cysteine residue by replacing a native amino acid residue with a cysteine residue. In some embodiments, the spin label is attached to the engineered cysteine residue of the apoA-II protein. In some embodiments, the apoA-II protein is a non-human apoA-II protein.

In some embodiments, the invention provides a composition comprising a spin-labeled lipoprotein probe with high specificity for HDL wherein the lipoprotein is an apoA-II protein. In some embodiments, the spin label comprises an atom that bears a free electron. In some embodiments, the atom bearing a free electron is a nitrogen atom. In some embodiments, the spin label is a nitroxide. In some embodiments, the spin label is selected from (1-oxyl-2,2,5,5-tetramethyl-Δ3-pyrroline-3-methyl)methanethiosulfonate; (1-oxyl-2,2,5,5-tetramethyl-Δ3-pyrroline-3-methyl)methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; and 3-(2-iodo-acetamido-methyl)-PROXYL, free radical. In some embodiments, the spin-label is a perdeuterated spin label. In some embodiments, the spin label is not (1-oxyl-2,2,5,5-tetramethyl-Δ3-pyrroline-3-methyl)methanethiosulfonate. In some embodiments the apoA-II protein is a human apoA-II protein.

In some aspects, the invention provides compositions comprising a spin-labeled lipoprotein probe with high specificity for HDL wherein the lipoprotein is an apoA-II protein. In some embodiments, the spin label is covalently attached to the lipoprotein. In some embodiments, the spin label is non-covalently attached to the lipoprotein. In some embodiments, the spin label associates with the lipoprotein. In some embodiments, the spin label is covalently attached to an amino acid residue on the lipoprotein. In some embodiments, the spin label is covalently attached to a cysteine residue on the lipoprotein. In some embodiments, the spin label is covalently attached to a cysteine residue on the lipoprotein through a thiosulfonate linkage.

In some embodiments, the invention provides compositions comprising a spin-labeled lipoprotein probe with high specificity for HDL wherein the lipoprotein is an apoA-II protein. In some embodiments, the spin label is covalently tethered to the lipoprotein by use of a spacer moiety between the spin label and the lipoprotein. Examples of spacer moieties include alkanes such as methane, ethane, propane, butane and the like. In some embodiments, the spin label is covalently attached to a lipoprotein through a methylthiosulfonate linkage, an ethylthiosulfonate linkage, a propylthiosulfonate linkage, or a butylthiosulfonate linkage. In some embodiments the apoA-II protein is a non-human mammalian apoA-II protein. In some embodiments the apoA-II protein is a human apoA-II protein.

In some embodiments, the invention provides compositions comprising a spin-labeled lipoprotein probe with high specificity for HDL, wherein the lipoprotein is an apoA-I mimetic. In some embodiments the apoA-I mimetic is a mimetic of a non-human mammalian apoA-I protein. In some embodiments the apoA-I mimetic is a mimetic of human apoA-I protein. In some embodiments, the apoA-I mimetic is 18A, 18A-Pro-18A, 4F and 4f-Pro-4F. In some embodiments, a spin label is covalently attached to the mimetic at a single site in the mimetic. ApoA-I mimetic 4F has the following amino acid sequence: Ac-DWFKAFYDKVAEKFKEAF-NH2 (SEQ ID NO:6) and 4F-Pro-4F is a tandem dimer of 4F connected by a proline residue (Wool, G D et al. (2009) J. Lipid Res. 50:1889-1900).

In some aspects, the invention provides a composition comprising a spin-labeled lipoprotein probe with high specificity for HDL wherein the lipoprotein is an apoA-I mimetic. In some embodiments, the spin label comprises an atom that bears a free electron. In some embodiments, the atom bearing a free electron is a nitrogen atom. In some embodiments, the spin label is a nitroxide. In some embodiments, the spin label is selected from (1-oxyl-2,2,5,5-tetramethyl-Δ3-pyrroline-3-methyl)methanethiosulfonate; (1-oxyl-2,2,5,5-tetramethyl-Δ3-pyrroline-3-methyl)methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; and 3-(2-iodo-acetamido-methyl)-PROXYL, free radical. In some embodiments, the spin-label is a perdeuterated spin label. In some embodiments the apoA-I mimetic is a mimetic of a non-human mammalian apoA-I protein. In some embodiments the apoA-I mimetic is a mimetic of human apoA-I protein.

In some aspects, the invention provides compositions comprising a spin-labeled lipoprotein probe with high specificity for HDL wherein the lipoprotein is an apoA-I mimetic. In some embodiments the apoA-I mimetic is a mimetic of a non-human mammalian apoA-I protein. In some embodiments the apoA-I mimetic is a mimetic of human apoA-I protein. In some embodiments, the spin label is covalently attached to the lipoprotein. In some embodiments, the spin label is non-covalently attached to the lipoprotein. In some embodiments, the spin label associates with the lipoprotein. In some embodiments, the spin label is covalently attached to an amino acid residue on the lipoprotein. In some embodiments, the spin label is covalently attached to a cysteine residue on the lipoprotein. In some embodiments, the spin label is covalently attached to a cysteine residue on the lipoprotein through a thiosulfonate linkage.

In some aspects, the invention provides compositions comprising a sample (e.g., a biological or synthetic sample as described herein) and a spin-labeled lipoprotein probe with high specificity for HDL wherein the lipoprotein is an apoA-I or fragment thereof, and the spin label is (1-oxyl-2,2,5,5-tetramethylpyrrolidin-3-yl)methyl methanesulfonate. In some embodiments, the invention provides compositions comprising a spin-labeled lipoprotein probe with high specificity for HDL wherein the lipoprotein is an apoA-I mimetic. In some embodiments the apoA-I mimetic is a mimetic of a non-human mammalian apoA-I protein. In some embodiments the apoA-I mimetic is a mimetic of human apoA-I protein. In some embodiments, the spin label is covalently tethered to the lipoprotein by use of a spacer moiety between the spin label and the lipoprotein. Examples of spacer moieties include alkanes such as methane, ethane, propane, butane and the like. In some embodiments, the spin label is covalently attached to a lipoprotein through a methylthiosulfonate linkage, an ethylthiosulfonate linkage, a propylthiosulfonate linkage, or a butylthiosulfonate linkage.

In some aspects, the invention provides compositions comprising an in vitro blood sample and a spin-labeled lipoprotein probe with high specificity for HDL wherein the lipoprotein is an apoA-I or fragment thereof, and the spin label is (1-oxyl-2,2,5,5-tetramethylpyrrolidin-3-yl)methyl methanesulfonate. In some embodiments, the apoA-I protein is a human apoA-I protein. In some embodiments, the apoA-I has the sequence of SEQ ID NO:1 or a fragment thereof. In some embodiments, the spin label is located at a single residue on the apoA-I protein or fragment thereof. In some embodiments, the apoA-I probe comprises two spin-labels, each at a single amino acid residue in the apoA-I protein. In some embodiments, the spin label is covalently attached to the apoA-I protein or fragment thereof. In some embodiments, the spin label is non-covalently attached or associated with the apoA-I protein or fragment thereof. In some embodiments the spin label is attached to a cysteine residue in the apoA-I protein. The native apoA-I protein does not contain a cysteine residue. In some embodiments of the invention, the apoA-I is engineered to contain a cysteine residue by replacing a native amino acid residue with a cysteine residue. This provides a means for specifically directing the spin label to a single site on the apoA-I protein with a reduced risk of generating a spin-labeled apoA-I protein in which a portion of the spin-labels are attached to the apoA-I protein in a random fashion. In some embodiments of the invention, the apoA-I protein is engineered to locate single cysteine residue at any site from residue 188 to residue 243. In some embodiments, the spin label is attached to the single cysteine residue genetically engineered at any site from residue 188 to residue 243. In some embodiments, the spin label is attached to a residue of apoA-I at any site from residue 188 to residue 243. In some embodiments of the invention, the spin label is attached to a cysteine genetically engineered to sites 98, 111 or 217 of the apoA-I protein. In some embodiments of the invention, the spin label is attached to a residue of the apoA-I protein to sites 98, 111 or 217 of the apoA-I protein. In some embodiments, the spin label is covalently attached to a cysteine residue at position 217 of the apoA-I protein. In some embodiments, the spin label is covalently attached to an amino acid at position 26, 44, 64, 101, 167, or 226 of the apoA-I lipoprotein. In some embodiments, the native amino acid residue at position 26, 44, 64, 101, 167, or 226 has been replaced by a cysteine residue. In some embodiments, the spin label is attached to residue 217 of the apoA-I protein. In some embodiments, the spin label is attached to a cysteine residue genetically engineered to site 217 of the apoA-I protein (SEQ ID NO:2). In some embodiments, the spin label is attached to residue 26 of the apoA-I protein. In some embodiments, the spin label is attached to a cysteine residue genetically engineered to site 26 of the apoA-I protein (SEQ ID NO:2). In some embodiments, the spin label is attached to residue 44 of the apoA-I protein. In some embodiments, the spin label is attached to a cysteine residue genetically engineered to site 44 of the apoA-I protein (SEQ ID NO:2). In some embodiments, the spin label is attached to residue 64 of the apoA-I protein. In some embodiments, the spin label is attached to a cysteine residue genetically engineered to site 64 of the apoA-I protein (SEQ ID NO:2). In some embodiments, the spin label is attached to residue 98 of the apoA-I protein. In some embodiments, the spin label is attached to a cysteine residue genetically engineered to site 98 of the apoA-I protein (SEQ ID NO:2). In some embodiments, the spin label is attached to residue 101 of the apoA-I protein. In some embodiments, the spin label is attached to a cysteine residue genetically engineered to site 101 of the apoA-I protein (SEQ ID NO:2). In some embodiments, the spin label is attached to residue 111 of the apoA-I protein. In some embodiments, the spin label is attached to a cysteine residue genetically engineered to site 111 of the apoA-I protein (SEQ ID NO:2). In some embodiments, the spin label is attached to residue 167 of the apoA-I protein. In some embodiments, the spin label is attached to a cysteine residue genetically engineered to site 167 of the apoA-I protein (SEQ ID NO:2). In some embodiments, the spin label is attached to residue 226 of the apoA-I protein. In some embodiments, the spin label is attached to a cysteine residue genetically engineered to site 226 of the apoA-I protein (SEQ ID NO:2). In some embodiments, the apoA-I protein is a non-human mammalian apoA-I protein.

In some aspects, the invention provides compositions comprising a sample (e.g., a biological or synthetic sample as described herein) and a spin-labeled lipoprotein probe with high specificity for HDL wherein the lipoprotein is an apoA-I or fragment thereof, and the spin label is (1-oxyl-2,2,5,5-tetramethylpyrrolidin-3-yl)methyl methanesulfonate. In some aspects, the invention provides compositions comprising an in vitro blood sample and a spin-labeled lipoprotein probe with high specificity for HDL wherein the lipoprotein is an apoA-I or fragment thereof, and the spin label is (1-oxyl-2,2,5,5-tetramethylpyrrolidin-3-yl)methyl methanesulfonate. In some embodiments, the spin label is covalently attached to the lipoprotein. In some embodiments, the spin label is non-covalently attached to the lipoprotein. In some embodiments, the spin label associates with the lipoprotein. In some embodiments, the spin label is covalently attached to an amino acid residue on the lipoprotein. In some embodiments, the spin label is covalently attached to a cysteine residue on the lipoprotein. In some embodiments, the spin label is covalently attached to a cysteine residue on the lipoprotein through a thiosulfonate linkage. In some embodiments the apoA-I protein is a non-human mammalian apoA-I protein.

In some aspects, the invention provides compositions comprising a sample (e.g., a biological or synthetic sample as described herein) and a spin-labeled lipoprotein probe with high specificity for HDL wherein the lipoprotein is an apoA-I or fragment thereof, and the spin label is (1-oxyl-2,2,5,5-tetramethylpyrrolidin-3-yl)methyl methanesulfonate. In some aspects, the invention provides compositions comprising an in vitro blood sample and a spin-labeled lipoprotein probe with high specificity for HDL wherein the lipoprotein is an apoA-I or fragment thereof, and the spin label is (1-oxyl-2,2,5,5-tetramethylpyrrolidin-3-yl)methyl methanesulfonate. In some embodiments, the spin label is covalently tethered to the lipoprotein by use of a spacer moiety between the spin label and the lipoprotein. Examples of spacer moieties include alkanes such as methane, ethane, propane, butane and the like. In some embodiments, the spin label is covalently attached to a lipoprotein through a methylthiosulfonate linkage, an ethylthiosulfonate linkage, a propylthiosulfonate linkage, or a butylthiosulfonate linkage. In some embodiments the apoA-I protein is a non-human mammalian apoA-I protein. In some embodiments the apoA-I protein is human apoA-I protein.

### Test Strips

In some aspects, the invention provides test strips for determining the capacity of HDL to support reverse cholesterol transport. In some embodiments, the invention provides compositions comprising a test strip, wherein the test strip comprises a spin-labeled lipoprotein probe and a solid support, wherein the spin-labeled lipoprotein probe comprises a spin label and a lipoprotein as described herein and wherein the spin-labeled lipoprotein probe has high specificity for HDL. In some embodiments, the strip is composed of either a polymer or cellulose base. In some embodiments the strip bears an inherent low EPR signature in the magnetic field range being observed (3000 to 4000 Gauss). In some embodiments, the test strip comprises a solid support. In some examples, the polymer can be either absorbent or an absorbent reagent pad is adhered to the polymer. For example, the absorbent reagent pad can be as simple as a cellulose strip or as complex as a hydrophilic polymer, wherein EPR spin probe and EPR reference standard are absorbed or covalently attached. Examples of materials that may be used for the test strip include but are not limited to polyvinylidene fluoride (PVDF), nylon or nitrocellulose. Materials for use in the test strip include commercially available adsorbent materials such as those commercially available from Millipore, Whatman or Pall. The test strip of the invention is designed for use in an EPR spectrometer. The test strip of the invention is not bound by any particular shape or size as long as it is suitable for use with an EPR spectrometer.

In some embodiments the reagent strip absorbs and immobilizes the EPR spin probe and the EPR reference standard and efficiently presents the probe to the human plasma sample. In some embodiments, a defined amount of EPR reference standard is impregnated onto either the polymer or absorbent reagent pad. In addition, the EPR spin probe may be impregnated, absorbed or adsorbed or into the polymer or absorbent reagent pad of the strip. In one embodiment of the invention, both the EPR reference standard and EPR spin probe are impregnated into the polymer or absorbent reagent pad. Somewhat parallel examples of similar strip compositions are glucose test strips or ketone test strips.

In some aspects, the invention provides an EPR spin probe comprising an apoA-I protein or HDL-specific peptide or polymer that bears a nitroxide spin label. The nitroxide probe may bear a different EPR spectrum when lipid-free versus HDL associated. The degree of difference is a measure of HDL function. In some embodiments, the material may be dried onto the test strip. In other embodiments, the material may be chemically adhered to its surface through covalent linkage. In other embodiments, the material may be chemically adhered to its surface through electrostatic linkage. In other embodiments, the material may be chemically adhered to its surface through hydrophobic linkage. In other embodiments, the material may be chemically adhered to its surface through any combination of covalent, electrostatic, and hydrophobic linkages.

The invention provides EPR reference standards which may be a paramagnetic stable radical that has an EPR spectra distinct from nitroxide probes that have similar properties to the nitroxide label on the EPR spin probe such that changes in its detection will be reflected in changes in the detection of the nitroxide probe. In some aspects, the purpose of the EPR reference standard is to enable normalization of the assay. For example, a well defined amount of EPR reference standard is impregnated into the polymer or absorbent reagent pad of the strip. In this example, the EPR reference standard allows the operator to calibrate the EPR instrument's dynamic range to a known response. Examples of EPR spin controls are: the tetramethylpiperidines (TEMPO, TEMPOL, TAMINE), TCNQ (tetracyanoquinodimethane), BZONO, SLPEO and similar variants. If a non-pyrroline nitroxide spin label is used for the EPR spin probe, a pyrroline-based spin label may be used as the reference standard. In some embodiments, the material may be dried onto the polymer or absorbent reagent pad of the strip or chemically adhered.

The instrument used to read the test strip is an EPR spectrometer fitted with an attachment that positions the EPR strip in a specific location within the EPR spectrometer's cavity. In some cases, the position of the strip within the instrument may be critical to examining a specific segment of the test strip and in a precise geometric location. In general, the instrument will detect signals in the X-band of the electromagnetic spectrum (7.0 to 12 GHz) and a magnetic field strength of 3000 to 4000 Gauss.

At least two modes of usage are envisioned. In the first nonlimiting example, the strips are impregnated with an EPR spin control reagent alone. An EPR probe is combined with a sample such as plasma (or other samples described herein) and administered to the strip in a specific volume. In some examples, the mixture is allowed to react on the strip at room temperature for 5 minutes (minimally) and inserted into the EPR spectrometer and the spectra obtained. In the second nonlimiting aspect of the invention, a specific amount of plasma is added to a test strip impregnated with EPR spin probe and EPR spin control and allowed to react on the strip at room temperature for 5 minutes. In both cases the EPR spin control may establish a relative signal intensity which will be used to calibrate the instrument. The signal from the EPR spin probe may be used to determine the relative response of the EPR spin probe to the HDL in the plasma.

In some embodiments of the invention, the test strips may also be used in the presence of a defined quantity of HDL modifying therapeutic (e.g., therapeutic compositions being tested for efficacy, diagnostic compositions being tested for sensitivity, etc). The therapeutic may be incorporated into the strip as the EPR spin probe or EPR reference standard are or is added to human plasma in a defined quantity and this is subsequently analyzed via EPR spectrometry on the strip.

### Biphasic Containers

In some aspects, the invention provides containers for use in the methods of the invention, wherein the interior of the container is biphasic. In some embodiments, the biphasic container comprises a solid material. In some embodiments the container contains a material for the capture of solids or solid-like materials in a sample to be used in the methods of the invention. In some embodiments, the invention provides biphasic containers where a sample to be used in the methods of the invention is added to the container and solids or solid-like materials in the sample are separated from one or more liquids in the sample. For example, the material can separate cells from plasma or serum. In some embodiments, the material inside the container binds, traps or otherwise segregates one or more solids or solid-like materials from one or more liquids in the sample.

In some embodiments, the material in the container is a space-filing material such as a filter, a mesh, a sponge, or a spongelike material. In some embodiments, the container comprises a solid zone and a liquid zone. In some embodiments, the material is cotton, cellulose, or a polymer such as vinyl. In some embodiments of the invention, the material in the container separates one or more solids in a sample from one or more liquids in the sample. In some embodiments, the material occupies a portion of the interior of the container. In some embodiments, the material occupies a first end of the container. In some embodiments, the material occupies a second end of the container. In some embodiments, the material occupies the middle of the container. In some embodiments the material is found throughout the interior of the container.

In some embodiments, the material is impregnated with an anti-coagulant. In some embodiments, the anti-coagulant is heparin, coumadin, warfarin, EDTA, citrate or oxalate.

In some embodiments the container comprises a spin-labeled lipoprotein probe. In some embodiments, the container comprises a drug. In some embodiments, the container comprises a spin-labeled lipoprotein probe and a drug. In further embodiments of the above embodiments, the spin-labeled lipoprotein probe and/or the drug are in a dry powdered form (e.g. lyophilized). In other embodiments of the above embodiments, the spin-labeled lipoprotein probe and/or the drug are in a liquid formulation. In some embodiments, the spin-labeled lipoprotein probe is added to the container before the sample is added to the container. In some embodiments, the spin-labeled lipoprotein probe is added to the container after the sample is added to the container. In some embodiments, the spin-labeled lipoprotein probe is added to the container at the same time as the sample is added to the container. In some embodiments, the spin-labeled lipoprotein probe is added to the sample before the sample is added to the container. In some embodiments, the drug is added to the container before the sample is added to the container. In some embodiments, the drug is added to the container after the sample is added to the container. In some embodiments, the drug is added to the container at the same time as the sample is added to the container. In some embodiments, the drug is added to the sample before the sample is added to the container.

In some embodiments, the container is a tube, a flatcell tube or a capillary tube. In some embodiments, the container is made of a non-paramagnetic material. In some embodiments, the container comprises glass, plastic, polymer or quartz. The interior of the tube can be any dimension. In some embodiments, the interior of the container is round. In some embodiments, the interior of the container is rectangular. In some embodiments of the invention, the interior of the container is flat rectangular. In some embodiments, the container is a single-bore container. In some embodiments, the container is a multi-bore container.

### Exemplary Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### EXAMPLES

The examples, which are intended to be purely exemplary of the invention and should therefore not be considered to limit the invention in any way, also describe and detail aspects and embodiments of the invention discussed above. Unless indicated otherwise, temperature is in degrees Centigrade and pressure is at or near atmospheric. The foregoing examples and detailed description are offered by way of illustration and not by way of limitation. All publications, patent applications, and patents cited in this specification are herein incorporated by reference as if each individual publication, patent application, or patent were specifically and individually indicated to be incorporated by reference. In particular, all publications cited herein are expressly incorporated herein by reference for the purpose of describing and disclosing compositions and methodologies which might be used in connection with the invention. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### Example 1

### Gel-Based Analysis of ApoA-I Binding/Displacement

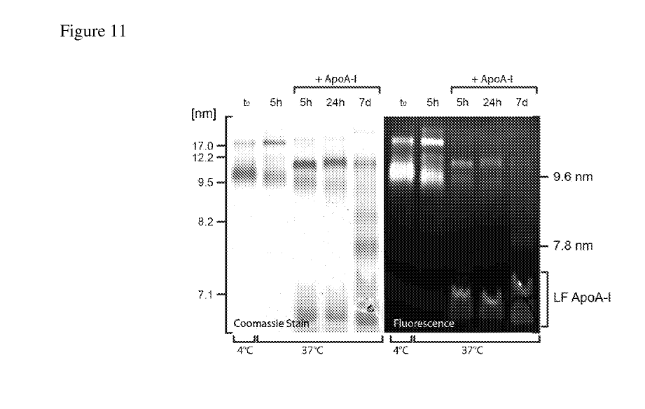

ApoA-I is a member of the exchangeable family of apolipoproteins, which is a class of proteins that can migrate from one lipoprotein pool to another and also exit the lipoprotein pool as a lipid-poor protein [50]. The exchange of apolipoproteins between lipoprotein particles is a central element of lipid metabolism. While there is significant evidence that the exchange of apoA-I is a displacement reaction, the direct binding and displacement of apoA-I from HDL particles has not been directly demonstrated. Whether resident apoA-I on HDL could be brought into equilibrium with exogenously added lipid-free/lipid-poor apoA-I was examined. Fluorescent rHDL were generated using an Alexa350 labeled apoA-I variant (labeled at position E136). Different sized rHDL particles (7.8, 8.4 and 9.6 nm) were reconstituted with Alexa350 labeled apoA-I and purified as previously described [51]. The purified rHDL were incubated with unlabeled lipid-free apoA-I in a protein:protein molar ratio of 1:5 at 37° C. for up to 7 days. The lipoproteins were examined by non-denaturing gradient gel electrophoresis (NDGGE). Within the first 5 hours, there was a rapid release of over 90% of the Alexa350 labeled apoA-I into the lipid-free protein pool (FIG. 11). The particles were stable up to 24 hours at 37° C. indicating that apoA-I binding/displacement from HDL was not due to disassembly or extensive remodeling of the HDL particles. Similar results were obtained when the exogenous lipid free apoA-I was Alexa 350 labeled [51]. The rate of apoA-I binding/displacement reflected the relative ability of that rHDL to efflux cholesterol via ABCA1. The 9.6 nm particle exhibited the slowest rate of apoA-I binding/displacement and the 7.8 nm particle exhibited the fastest rate of apoA-I binding/displacement. The 7.8 nm particle is the preferred substrate for ABCA1 mediated cholesterol efflux. This process of apoA-I displacement is significantly accelerated (˜5 min) by the presence of plasma factors [41, 52] such as cholesteryl ester transfer protein (CETP) [53], lecithin:cholesterol acyltransferase (LCAT) [54, 55], phospholipid transfer protein (PLTP) [56, 57] and hepatic lipase [58].

### Example 2

### FRET-Based Assay of ApoA-I Exchange

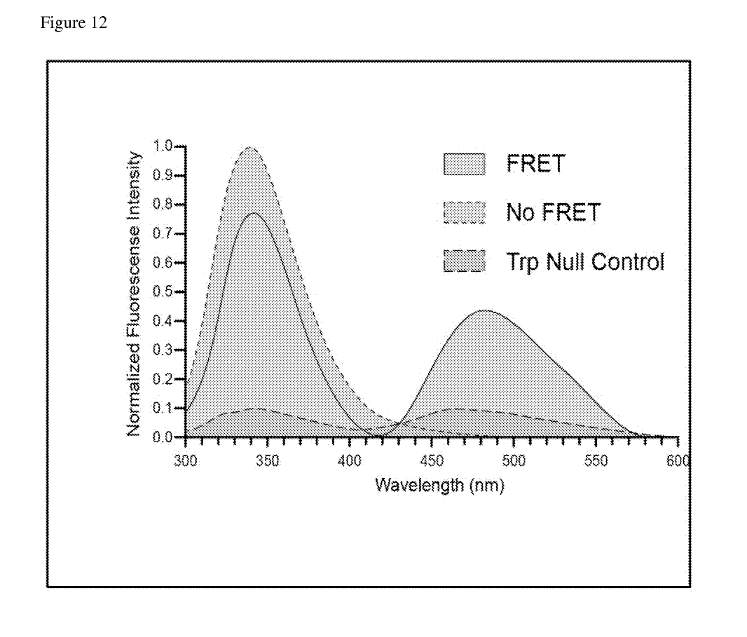

While gel-based evaluation of apoA-I binding/displacement is informative, the timescale and resolution of this approach is unable to resolve complex differences in binding/displacement kinetics resulting from alterations in oxidation state and HDL particle composition. To address this shortcoming of the gel-based approach, a fluorescence resonance energy transfer (FRET)-based assay was developed based on the apoA-I conformation in lipid-free and lipid-bound states. FRET is a powerful technique that can determine the inter-residue distance within a protein that is useful for deducing the conformational state of a protein if residues are proximal in one conformation and distal in another. The effective range of FRET is 10-75 Å, which is well suited for the dimensions of lipid-free versus lipid-bound apoA-I. Atomic distance is measured by the degree of energy exchange from a donor fluorophore to an acceptor fluorophore. The fluorescence characteristics of tryptophan was utilized, whose emission spectra (330 to 350 nm) overlaps the absorption spectra of N-iodoacetyl-N′-(5-sulfo-1-napthyl)ethylenediamine (AEDANS). An apoA-I variant was created wherein the four endogenous tryptophans were substituted for phenylalanine (Trp Null apoA-I). These Trp to Phe substitutions in apoA-I do not significantly affect protein structure [59] or function, as demonstrated by comparable ABCA1-mediated cholesterol efflux activity of WT [60]. The distance between the tryptophan fluorescent donor to the AEDANS fluorescent acceptor moiety was determined by the relative levels of AEDANS fluorescence at 440 nm (FIG. 12), which is representative of the degree of energy transfer.

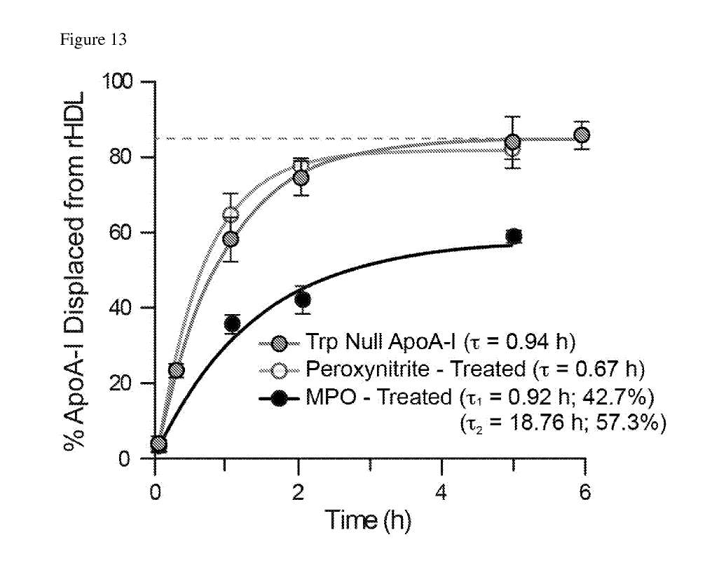

Previous investigations of apoA-I structure in lipid-free [61-63] and lipid-bound states [64, 65] indicated that amino acid positions 19 and 136 are proximal in the structure of lipid-free apoA-I and distal in the lipid bound apoA-I structure (FIGS. 12A and B). To measure the rate of opening of the lipid-free apoA-I bundle into an extended helix on discoidal HDL, Trp was substituted for a Val at position 19 in Trp Null apoA-I (apoA-IW19) and a Cys was substituted for a Glu at position 136. The absence of endogenous cysteines within apoA-I was utilized to label the introduced cysteine with AEDANS, utilizing maleimide thiol chemistry. The resultant apoA-IW19:A136 was used to generate rHDL. Similar to the gel-based assay described in Example 1, fluorescently labeled rHDL were incubated with unlabeled apoA-I (Trp Null apoA-I) in a 1:5 protein:protein molar ratio. The displacement of apoA-IW19:A136 from rHDL was observed as an increase in AEDANS fluorescence at λmax (440 nm). From this the rate of apoA-I binding/displacement, termed the “exchange rate”, was determined. The emission spectrum of the exchange reaction mixture at 0 h was used as a reference for 0% exchange and the spectrum at 72 h as the final equilibrium state (maximal exchange; average of 6 experiments) (FIG. 13). Mono-exponential data-fitting analysis yielded an exponential relaxation time (time to 50% maximal exchange, τ) of 0.94 h, a measure consistent with the qualitative evaluation of apoA-I binding/displacement observed by NDGGE (FIG. 11). The emission spectra of fluorescently labeled rHDL, when incubated alone, showed no changes during the same time period, further indicating that changes in fluorescence emission spectra are not a result of spontaneous remodeling of rHDL.

### Example 3

### Effect of Oxidation on Exchange Rates

The effect of oxidation by peroxynitrite and MPO on apoA-I's rate of exchange. Lipid-free Trp Null apoA-I was subjected to oxidation by the MPO—H₂O₂-nitrite system, a potent source of reactive nitrogen species [66]. Lipid-free Trp Null apoA-I was also subjected to oxidation by peroxynitrite. The distinction between these two modes of oxidation is that MPO-mediated oxidation is a potent source of 3-chlorotyrosine and 3-nitrotyrosine, which severely reduce the ability of apoA-I to efflux cholesterol by ABCA1 [28, 30], whereas peroxinitrite oxidation of apoA-I does not lead to a significant decline in apoA-I's ABCA1-mediated efflux capacity [67]. When the effect of peroxynitrite oxidation was tested, no significant differences in the rate of apoA-I exchange were observed (FIG. 5) [38]. In contrast MPO oxidation leads to two populations of apoA-I, one with a normal degree of exchange and a second population (57%) severely impaired in its ability to exchange with HDL-resident apoA-I [38]. Interestingly, MPO oxidation of apoA-I under similar conditions led to a notably comparable reduction (˜50%) in apoA-I's ability to facilitate cholesterol efflux by ABCA1 [31]. These data suggest that the apoA-I exchange rate of HDL (otherwise referred to apoA-I HDL binding/displacement) is reflective of its cholesterol efflux capacity.

The FRET-based assay has gives insight into the effects of oxidation on HDL function. Oxidative reactions that impair apoA-I exchange also inhibit apoA-I's ability to efflux cholesterol via ABCA1, whereas oxidative reactions that do not affect apoA-I exchange also do not impair ABCA1 mediated cholesterol efflux. The potential CAD predictive value of measuring apoA-I exchange rates is further validated by the fact that the chemical modifications that impair apoA-I's exchange rate are similar to those observed in diabetes [29, 68, 69], obesity, and tobacco smoking [70-72], which are associated with increased incidence of CAD.

### Example 4

### EPR Methodology

Using EPR the structure of apoA-I in lipid-free and lipid-bound states have been examined. The EPR solution to apoA-I's N-terminal structure on 9.6 nm reconstituted discoidal HDL [65]. This methodology is analogous to NMR, and provides information on the structural micro-environment of the spin-label (˜10-15 Å). Specifically, the conformation of this region can be derived from three principal parameters measurable by EPR: peptide backbone mobility (FIG. 5), solvent accessibility of the spin-label, and relative fluidity of the environment. The later is most applicable to examining membrane associated proteins and the fluidity of the proximal lipids. Hubbell and co-workers have characterized modulations in EPR spectral line-shapes and have identified specific protein structural characteristics associated with these changes [73, 74]. From this structural conclusions may be drawn from the shape of the EPR spectra of spin-labeled sites in proteins. Therefore, if a spin label is positioned in portion of apoA-I that bears a unique conformation in the lipid-free versus lipid bound state, the EPR spectra can be used to distinguish between these two forms of apoA-I.

Binding of ApoA-I to Human Plasma HDL.

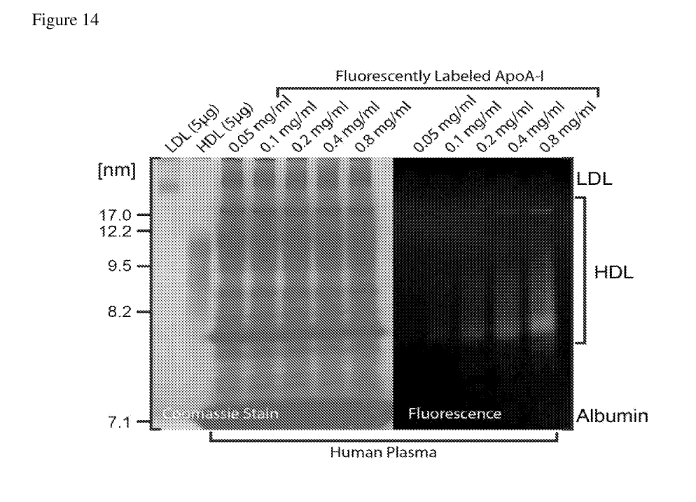

The Alexa350 labeled apoA-I that had previously been used to evaluate apoA-I binding/displacement from HDL (FIG. 11) was used to investigate the binding preference of lipid-free apoA-I in human plasma. To heparinized human plasma from a healthy fasted female volunteer donor, Alexa350 labeled apoA-I was added to a concentration of 0.05, 0.1, 0.2, 0.4, and 0.8 mg/ml. The plasma with exogenous apoA-I was incubated at 37° C. for 2 hours and resolved by NDGGE (FIG. 14). The primary binding of apoA-I is limited to the HDL lipoprotein fraction. A significant portion of apoA-I is associated with a particle approximately 8.0 nm in diameter, although the apoA-I is also bound to larger HDL particles. There is some apoA-I present in a region of the gel that corresponds to preβ HDL. An important observation made in this experiment is that apoA-I added to human plasma cannot be found in either the VLDL (extreme top), LDL, or albumin portions of the gel. Because apoA-I co-migrates with the HDL lipoprotein fraction on NDGGE and apoA-I has a reportedly high specificity for HDL [39], it is likely that apoA-I is binding to HDL and the data gathered from the spin label are reporting aspects of HDL.

### Example 5

### Examination of Human Plasma HDL by EPR

To evaluate the feasibility of employing EPR as a means of directly assessing HDL in plasma, the following are examined: 1) the sensitivity of newly improved EPR instrumentation for this analysis; 2) the specificity of apoA-I for plasma HDL; and 3) whether EPR spectra would reveal differences in human plasma samples collected from normal and patients “at risk” for CAD. Initially it was tested whether the enhanced sensitivity of the JEOL TE-100 EPR spectrometer was sufficient to measure apoA-I's structural characteristics at physiologically relevant concentrations. ApoA-I reference samples (apoA-I spin labeled at a variety of well characterized locations) were evaluated at decreasing concentration until a reproducible signal could not be detected. This threshold was at 0.1 mg/ml, well below the physiological concentration of apoA-I in plasma. For these studies, 0.3 mg/ml spin labeled apoA-I was used as it provides the optimal degree of HDL specificity and bears a robust and reproducible signal on the EPR spectrometer.

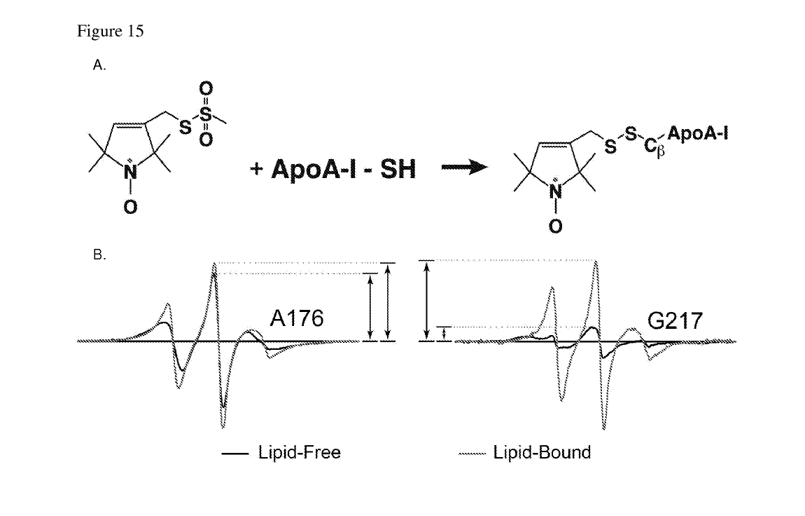

ApoA-I is labeled using cysteine substituted variants, taking advantage of the absence of endogenous cysteines within apoA-I to position the label at specific locales (FIG. 15A). Because this assay relies on the ability to discriminate between lipid-free and lipid-bound apoA-I, position G217 in apoA-I is chosen as the spin label site. The structure of apoA-I at 240 of apoA-I's 243 amino acids have been examined and the effect of lipid association on apoA-I structure down the entire length of the protein has been determined. The EPR spectra of residue G217 is affected by lipid binding (FIG. 15B). This shift in EPR spectra upon lipid binding serves as a very sensitive reporter for HDL association. It also serves as an indicator of apoA-I conformation at that position within apoA-I. In contrast, position A176 is not significantly affected by lipid and thus, like a majority of apoA-I residues, is a poor location for reporting lipid binding. While the dynamic range of response is the largest at G217, other residues are comparably altered upon lipidation and could also serve as reporter locations. ApoA-I that has been spin labeled at position G217 (apoA-ISL217) bear nearly identical structural properties as WT apoA-I and efflux cholesterol and form HDL in a fashion indistinguishable from WT apoA-I. To quantify binding, the maximum amplitude of the center EPR peak relative to lipid bound apoA-I is measured (FIG. 15, arrows). This is a reliable measure of the degree of lipid association.

### Example 6

### Comparison of EPR Spectra of Human Plasma from Healthy and at Risk Patients

To investigate whether EPR spectroscopy is a feasible approach to evaluate the quality of a patient's HDL directly in plasma, the EPR spectra of apoA-ISL217 in the plasma of 4 patients was compared (Table 2). Two individuals were characterized as having normal lipid and CAD risk profiles and two other individuals were characterized as at high risk for CAD. Patients were matched patients based on their concentrations of HDL-C and apoA-I to aid in interpreting results.

TABLE 2
Patient Data

| | Statin | Diagnosed | | | | |
|---|---|---|---|---|---|---|
| Patient | Treatment | as Diabet- | LDL | HDL | TG | |
| ID | (y/n) | ic (y/n) | (mg/dL) | (mg/dL) | (mg/dL) | BMI |
| N1 | N | N | 90 | 43 | 90 | 21.5 |
| N2 | N | N | 100 | 41 | 110 | 24.3 |
| D1 | Y | Y | 83 | 42 | 182 | 46.6 |
| M1 | N | N | 138 | 40 | 130 | 47.5 |

Samples were obtained from existing plasma banks at CHOR1. All confidentiality and human safety issues were observed during their collection. Patients were matched based on sex, relative HDL levels, and draw/storage method. Plasma were collected into heparinized tubes and frozen only once prior to analysis. Sample quality control was closely scrutinized to ensure that differences in sample collection and handling minimally contributed to the results.

In the assay, apoA-ISL217 at a concentration of 6.3 mg/ml was added to plasma in a 1:20 ratio (v/v), yielding 0.3 mg/ml apoA-ISL217 in plasma. The sample was immediately examined in the JEOL TE-100 EPR spectrometer and monitored at 1.5, 4, 6, 8, and 10 minutes. Earlier EPR-based investigation determined that the rate of apoA-I binding to plasma HDL was rapid due to the presence of remodeling enzymes in plasma [41, 52-58]. Maximal association for all samples happened within 10 minutes (confirmed by lack of spectral change after 4 hours). Significant differences were observed between patient N1, who has a very healthy lifestyle and lipid profile, and patients D1 and M1, who are both morbidly obese with diabetes and metabolic syndrome, respectively. ApoA-ISL217 rapidly bound the HDL of patient N1's plasma within the first 5 minutes, in patients D1 and M1, binding was much slower and was maximal after 10 and 8 minutes, respectively (FIG. 8). The degree of maximal HDL binding was also similarly affected, patient N1 exhibited the maximal capacity for apoA-I binding and D1 the lowest. This was not a function of available HDL, because increases in apoA-ISL217 concentration in the assay yielded a similar extent of response, until saturating concentrations of apoA-ISL217 were achieved. That the saturating concentration of apoA-ISL217 was comparable for all individuals (˜1.9 mg/ml) indicates that the different responses observed were not due to saturation of HDL in patient plasma.

It was noted that patient N2's data appeared to have a low risk for CAD but the binding rate and degree of response were intermediate between patient D1 and M1. The family history of this individual was available and at least one parent and two grandparents suffered from incidences of CAD. This patient had not anticipated observing an “at risk” response to this assay in this individual based on available clinical indices. The family history, however suggests that a possible genetic component may exist and be detectable before any clinical signs of CAD may manifest. In contrast patient N1 does not have a family history of heart disease. While not a large enough study to be statistically significant, this outcome indicates the potential to become a biomarker for CAD risk even in the absence of overt clinical signs.

The effects of sample handling and collection procedure on the assay results. The effect of three modes of sample collection: heparin, citrate and EDTA was but no difference was observed between the methods. Because freeze-thaw handling can significantly affect HDL activities like enzyme and receptor interactions, the effect of repeated freeze-thaw cycles on the assay was examined. Freeze-thaw cycle reduced both the rate and degree of apoA-I binding by approximately 10%. Interestingly, after the third freeze-thaw this effect increased to approximately 20%. It was noted that plasma samples with higher TG concentration were more susceptible to freeze-thaw changes with decreases in EPR observable binding of 15 and 25%, respectively.

### Example 7

### Comparison of EPR Spectra from C57Bl/6 Mice and CH3 Mice

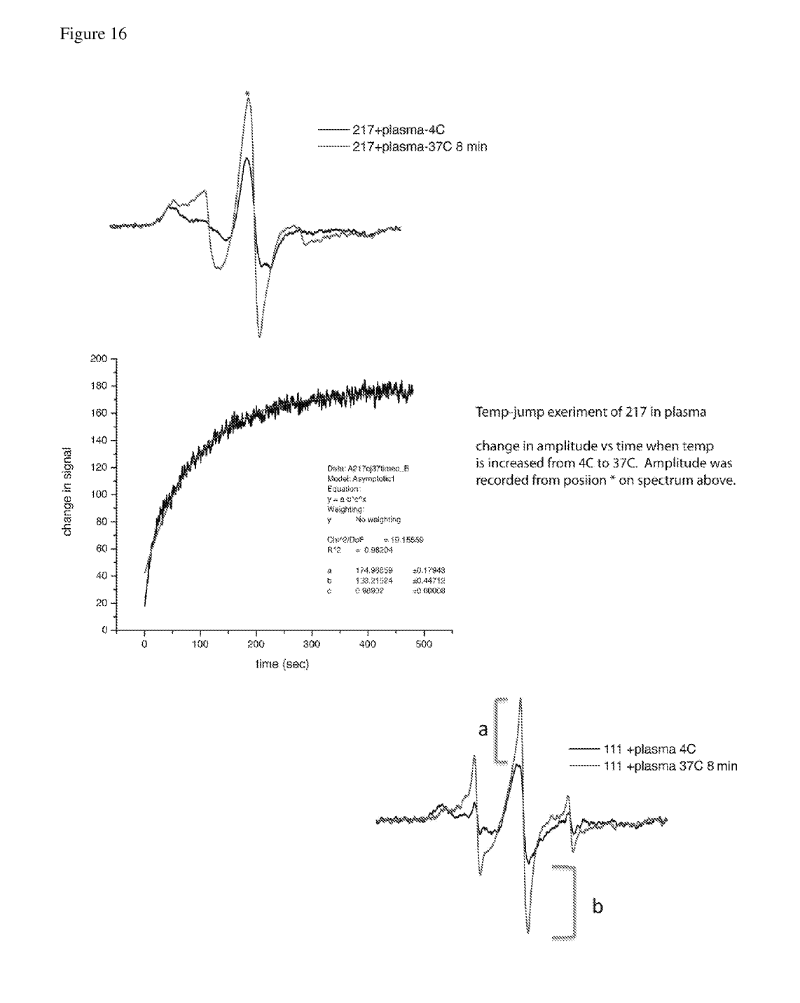

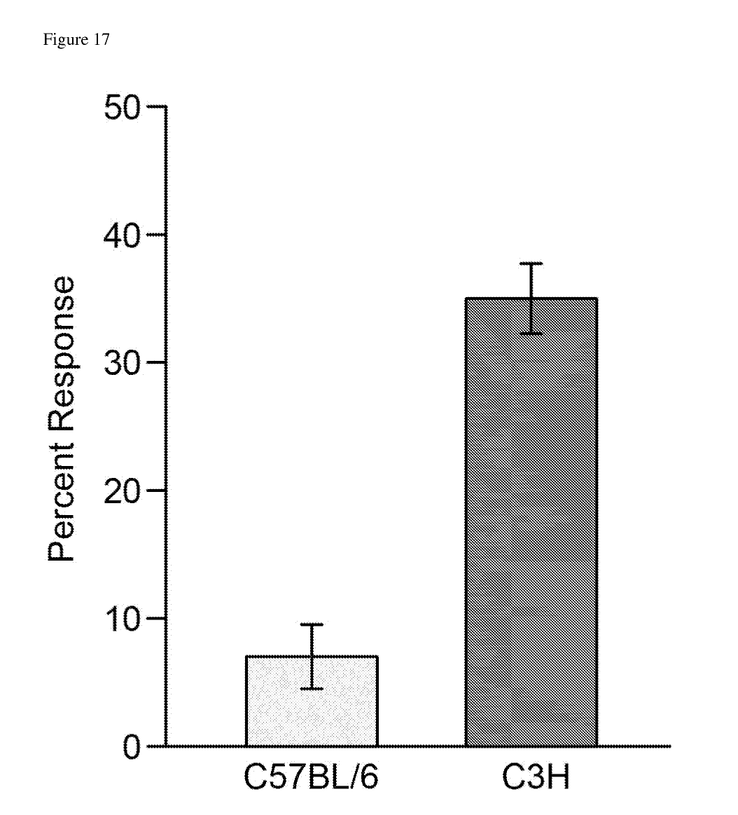

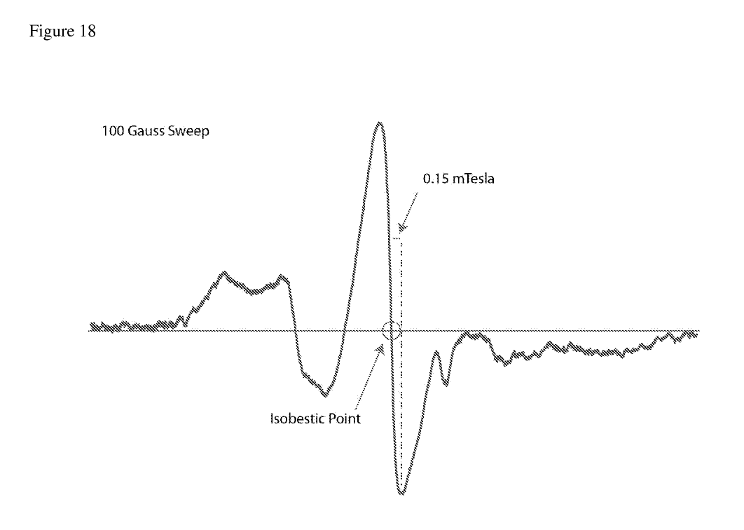

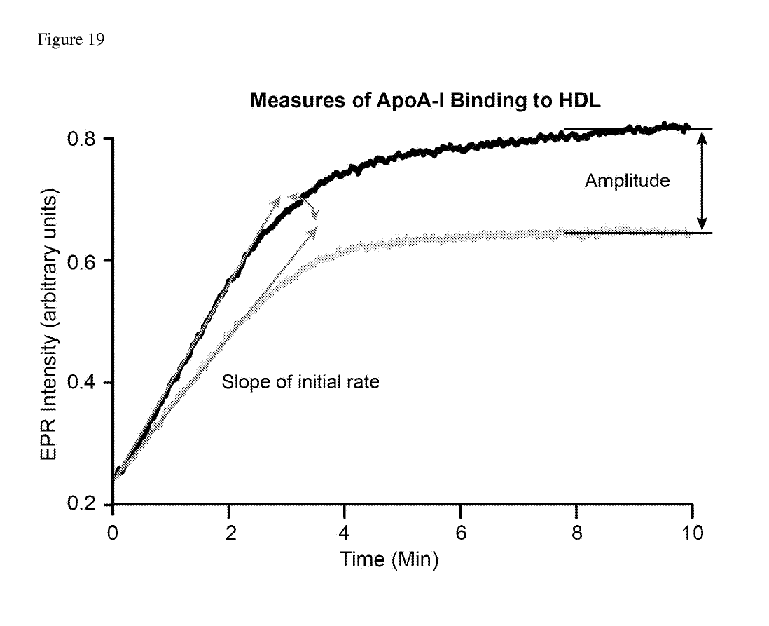

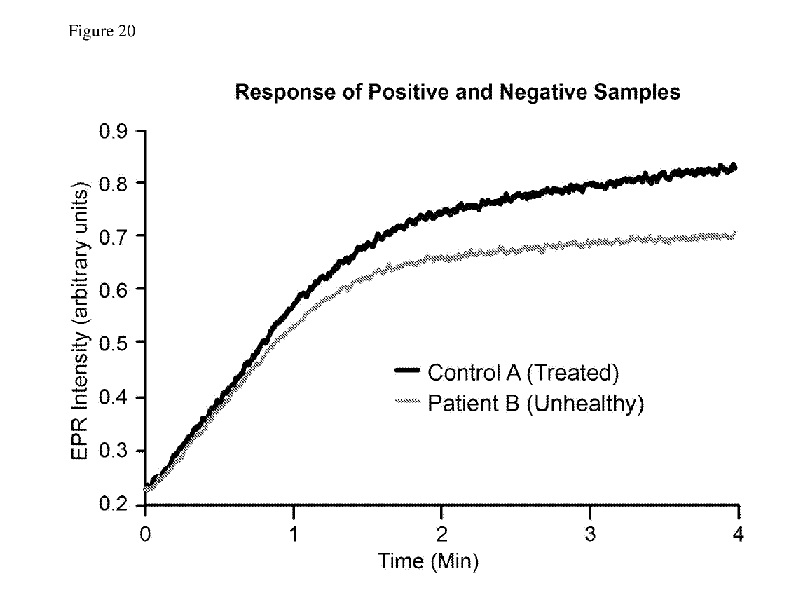

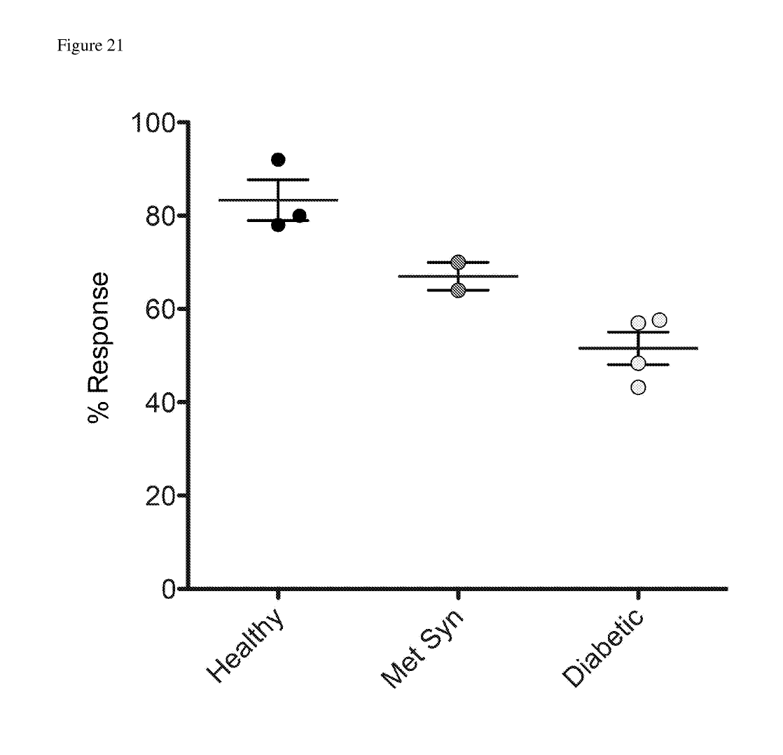

C57Bl/6 mice are genetically normal but prone to heart disease whereas C3H mice are genetically normal but not prone to heart disease. To assess the ability to apoA-ISL217 to identify reduced capacity of HDL for reverse cholesterol transport, plasma from these two strains were analyzed. C57Bl/6 mice were fed a normal diet whereas C3H mice were fed a high-fat diet. Plasma samples were removed from mice, the apoA-ISL217 was added to the plasma at a final concentration of 1.4 mg/ml and immediately the EPR spectra were collected. Collection was started, first at 4° C. to provide a baseline and then the temperature was shifted to 37° C. and spectra were collected continuously for up to 300 seconds. Sample spectra are shown in FIG. 16. Results are presented graphically in FIG. 17. Plasma samples from C57Bl/6 mice showed reduced binding to the spin-labeled probe compared to plasma samples from C3H mice. It is noted that one C3H mice was an extreme outlier and was not included in the analysis. The reason for this outlier in not known. It is also noted that response times for both mouse strains was low, most likely reflecting the use of a human apoA-I probe in mouse plasma.

A second probe, with a spin label at position 111, was used with some samples (FIG. 16, bottom panel) showing the utility of a spin label at position 111 to detect changes in apoA-I structure upon binding to lipid.

### Example 8

### EPR Spectral Position for Monitoring apoA-I Binding to HDL

The EPR spectral position for monitoring apoA-I binding to HDL was determined by identifying the magnetic strength of the isobestic point. 150 amount of spin labeled lipoprotein probe (apoA-ISL217 at 3 mg/ml) in PBS was added to 450 sample of human plasma in a flatcell sample holder. The sample was kept at 4° C. and a 100 gauss sweep of EPR signal was obtained (over 2 minutes; in the X-band). To determine the position for monitoring the binding of the spin-labeled lipoprotein probe a position 0.15 mTesla upfield of the isobestic point was identified. The peak approximately 0.15 mTesla (1.5 Gauss) up field of the isobestic point was observed continuously and the response of this position monitored with time.

### Example 9

### ApoA-I Binding to HDL

ApoA-I binding to HDL in human plasma was determined by continuously monitoring a spectral position approximately 0.15 mTesla (1.5 Gauss) up field of the isobestic point. 150 amount of spin labeled lipoprotein probe (apoA-ISL217 at 3 mg/ml) in PBS was added to 450 sample of plasma. The sample was kept at 4° C. and shifted to 37° C. As the temperature increased the position approximately 0.15 mTesla upfield of the isobestic point was continuously monitored for 10 minutes. From this analysis multiple parameters of apoA-I binding to HDL were discernible, namely the amplitude of the response and the slope of the initial rate of binding. A higher amplitude and greater slope of initial rate of binding were associated with greater efflux capacity. The high responder was plasma from a healthy individual. The low responder was plasma from an unhealthy individual.

### Example 10

### Response of Positive and Negative Samples

Traces of apoA-I binding to HDL in control human plasma samples. 15 μl amount of spin labeled lipoprotein probe (apoA-ISL217 at 3 mg/ml) in PBS was added to 45 μl sample of plasma. The sample was kept at 4° C. and shifted to 37° C. As the temperature increased the position approximately 0.15 mTesla upfield of the isobestic point was continuously monitored for 4 minutes. The cholesterol efflux capacity of human plasma controls A and B were determined prior to the experiment by cell based macrophage efflux measurements. Control A had a cholesterol efflux capacity 50% that of Control B. Similarly, Control B contained approximately twice the level of preBeta HDL as Control A.

### Example 11

### Reverse Cholesterol Transport Capacity of Plasma from Normal, Metabolic Syndrome and Diabetic Individuals

The plasma from 9 individuals whose diabetic/metabolic syndrome status had been identified were examined by the HDL-function assay. Briefly, blood plasma was collected from a set of fasted individuals (5 females and 4 males), whose diabetic status was well characterized. 45 μl of plasma was added to 15 μl of apoA-I probe (3 mg/ml). The apoA-I probe was composed of an apoA-I that bears a G217C mutation. This mutation introduced a cysteine into apoA-I, whose native sequence has no cysteine residues. The sulfhydril of the introduced cysteine (at position 217) was derivatized with a thiosulfonate linked nitroxide spin label. The resultant spin-labeled protein was concentrated 3 mg/ml. After addition of probe to plasma, the EPR signature spectra was monitored at both 8° C. and 37° C. The amplitude of the center field peak was reported as % response, relative to a reference sample. In this case the reference sample was the response of the probe to 0.1% SDS, which yielded a maximal lipid-like response. The patients were age matched. An internal standard (Bruker Proprietary Internal Standard) was included in the read (not shown) and used to control for instrument performance. But internal standards that such as manganese chloride will suffice.

### REFERENCES CITED

1. Gordon, T., W. P. Castelli, M. C. Hjortland, W. B. Kannel, and T. R. Dawber, High density lipoprotein as a protective factor against coronary heart disease. The Framingham Study. Am J Med, 1977. 62(5): p. 707-14.
2. Assmann, G., H. Schulte, A. von Eckardstein, and Y. Huang, High-density lipoprotein cholesterol as a predictor of coronary heart disease risk. The PROCAM experience and pathophysiological implications for reverse cholesterol transport. Atherosclerosis, 1996. 124 Suppl: p. S11-20.
3. Sharrett, A. R., C. M. Ballantyne, S. A. Coady, G. Heiss, P. D. Sorlie, D. Catellier, and W. Patsch, Coronary heart disease prediction from lipoprotein cholesterol levels, triglycerides, lipoprotein(a), apolipoproteins A-I and B, and HDL density subfractions: The Atherosclerosis Risk in Communities (ARIC) Study. Circulation, 2001. 104(10): p. 1108-13.
4. Gordon, D. J., J. L. Probstfield, R. J. Garrison, J. D. Neaton, W. P. Castelli, J. D. Knoke, D. R. Jacobs, Jr., S. Bangdiwala, and H. A. Tyroler, High-density lipoprotein cholesterol and cardiovascular disease. Four prospective American studies. Circulation, 1989. 79(1): p. 8-15.
5. Downs, J. R., M. Clearfield, S. Weis, E. Whitney, D. R. Shapiro, P. A. Beere, A. Langendorfer, E. A. Stein, W. Kruyer, and A. M. Gotto, Jr., Primary prevention of acute coronary events with lovastatin in men and women with average cholesterol levels: results of AFCAPS/TexCAPS. Air Force/Texas Coronary Atherosclerosis Prevention Study. JAMA, 1998. 279(20): p. 1615-22.
6. Rubin, E. M., R. M. Krauss, E. A. Spangler, J. G. Verstuyft, and S. M. Clift, Inhibition of early atherogenesis in transgenic mice by human apolipoprotein AI. Nature, 1991. 353(6341): p. 265-7.
7. Liu, A. C., R. M. Lawn, J. G. Verstuyft, and E. M. Rubin, Human apolipoprotein A-I prevents atherosclerosis associated with apolipoprotein[a] in transgenic mice. J Lipid Res, 1994. 35(12): p. 2263-7.
8. Hughes, S. D., J. Verstuyft, and E. M. Rubin, HDL deficiency in genetically engineered mice requires elevated LDL to accelerate atherogenesis. Arterioscler Thromb Vasc Biol, 1997. 17(9): p. 1725-9.
9. Voyiaziakis, E., I. J. Goldberg, A. S. Plump, E. M. Rubin, J. L. Breslow, and L. S. Huang, ApoA-I deficiency causes both hypertriglyceridemia and increased atherosclerosis in human apoB transgenic mice. J Lipid Res, 1998. 39(2): p. 313-21.
10. Asztalos, B. F., L. A. Cupples, S. Demissie, K. V. Horvath, C. E. Cox, M. C. Batista, and E. J. Schaefer, High-density lipoprotein subpopulation profile and coronary heart disease prevalence in male participants of the Framingham Offspring Study. Arterioscler Thromb Vasc Biol, 2004. 24(11): p. 2181-7.
11. Tsai, M. Y., C. Johnson, W. H. Kao, A. R. Sharrett, V. L. Arends, R. Kronmal, N. S. Jenny, D. R. Jacobs, Jr., D. Arnett, D. O'Leary, and W. Post, Cholesteryl ester transfer protein genetic polymorphisms, HDL cholesterol, and subclinical cardiovascular disease in the Multi-Ethnic Study of Atherosclerosis. Atherosclerosis, 2008. 200(2): p. 359-67.
12. Pedersen, T. R., O. Faergeman, J. J. Kastelein, A. G. Olsson, M. J. Tikkanen, I. Holme, M. L. Larsen, F. S. Bendiksen, C. Lindahl, M. Szarek, and J. Tsai, High-dose atorvastatin vs usual-dose simvastatin for secondary prevention after myocardial infarction: the IDEAL study: a randomized controlled trial. JAMA, 2005. 294(19): p. 2437-45.
13. van der Steeg, W. A., I. Holme, S. M. Boekholdt, M. L. Larsen, C. Lindahl, E. S. Stroes, M. J. Tikkanen, N. J. Wareham, O. Faergeman, A. G. Olsson, T. R. Pedersen, K. T. Khaw, and J. J. Kastelein, High-density lipoprotein cholesterol, high-density lipoprotein particle size, and apolipoprotein A-I: significance for cardiovascular risk: the IDEAL and EPIC-Norfolk studies. J Am Coll Cardiol, 2008. 51(6): p. 634-42.
14. Ross, R. and J. A. Glomset, Atherosclerosis and the arterial smooth muscle cell: Proliferation of smooth muscle is a key event in the genesis of the lesions of atherosclerosis. Science, 1973. 180(93): p. 1332-9.
15. Miller, N. E. and G. J. Miller, Letter: High-density lipoprotein and atherosclerosis. Lancet, 1975. 1(7914): p. 1033.
16. Terasaka, N., N. Wang, L. Yvan-Charvet, and A. R. Tall, High-density lipoprotein protects macrophages from oxidized low-density lipoprotein-induced apoptosis by promoting efflux of 7-ketocholesterol via ABCG1. Proc Natl Acad Sci USA, 2007. 104(38): p. 15093-8.
17. Cui, D., E. Thorp, Y. Li, N. Wang, L. Yvan-Charvet, A. R. Tall, and I. Tabas, Pivotal advance: macrophages become resistant to cholesterol-induced death after phagocytosis of apoptotic cells. J Leukoc Biol, 2007. 82(5): p. 1040-50.
18. Terasaka, N., S. Yu, L. Yvan-Charvet, N. Wang, N. Mzhavia, R. Langlois, T. Pagler, R. Li, C. L. Welch, I. J. Goldberg, and A. R. Tall, ABCG1 and HDL protect against endothelial dysfunction in mice fed a high-cholesterol diet. J Clin Invest, 2008. 118(11): p. 3701-13.
19. Spieker, L. E., I. Sudano, D. Hurlimann, P. G. Lerch, M. G. Lang, C. Binggeli, R. Corti, F. Ruschitzka, T. F. Luscher, and G. Noll, High-density lipoprotein restores endothelial function in hypercholesterolemic men. Circulation, 2002. 105(12): p. 1399-402.
20. Packard, C. J., D. S. O'Reilly, M. J. Caslake, A. D. McMahon, I. Ford, J. Cooney, C. H. Macphee, K. E. Suckling, M. Krishna, F. E. Wilkinson, A. Rumley, and G. D. Lowe, Lipoprotein-associated phospholipase A2 as an independent predictor of coronary heart disease. West of Scotland Coronary Prevention Study Group. N Engl J Med, 2000. 343(16): p. 1148-55.
21. Ridker, P. M., M. Cushman, M. J. Stampfer, R. P. Tracy, and C. H. Hennekens, Inflammation, aspirin, and the risk of cardiovascular disease in apparently healthy men. N Engl J Med, 1997. 336(14): p. 973-9.
22. Kuller, L. H., R. P. Tracy, J. Shaten, and E. N. Meilahn, Relation of C-reactive protein and coronary heart disease in the MRFIT nested case-control study. Multiple Risk Factor Intervention Trial. Am J Epidemiol, 1996. 144(6): p. 537-47.
23. Koenig, W., M. Sund, M. Frohlich, H. G. Fischer, H. Lowel, A. Doring, W. L. Hutchinson, and M. B. Pepys, C-Reactive protein, a sensitive marker of inflammation, predicts future risk of coronary heart disease in initially healthy middle-aged men: results from the MONICA (Monitoring Trends and Determinants in Cardiovascular Disease) Augsburg Cohort Study, 1984 to 1992. Circulation, 1999. 99(2): p. 237-42.
24. Daugherty, A., J. L. Dunn, D. L. Rateri, and J. W. Heinecke, Myeloperoxidase, a catalyst for lipoprotein oxidation, is expressed in human atherosclerotic lesions. J Clin Invest, 1994. 94(1): p. 437-44.
25. Hazen, S. L., J. P. Gaut, J. R. Crowley, F. F. Hsu, and J. W. Heinecke, Elevated levels of protein-bound p-hydroxyphenylacetaldehyde, an amino-acid-derived aldehyde generated by myeloperoxidase, are present in human fatty streaks, intermediate lesions and advanced atherosclerotic lesions. Biochem J, 2000. 352 Pt 3: p. 693-9.
26. Zheng, L., M. Settle, G. Brubaker, D. Schmitt, S. L. Hazen, J. D. Smith, and M. Kinter, Localization of nitration and chlorination sites on apolipoprotein A-I catalyzed by myeloperoxidase in human atheroma and associated oxidative impairment in ABCA1-dependent cholesterol efflux from macrophages. J Biol Chem, 2005. 280(1): p. 38-47.
27. Bergt, C., S. Pennathur, X. Fu, J. Byun, K. O'Brien, T. O. McDonald, P. Singh, G. M. Anantharamaiah, A. Chait, J. Brunzell, R. L. Geary, J. F. Oram, and J. W. Heinecke, The myeloperoxidase product hypochlorous acid oxidizes HDL in the human artery wall and impairs ABCA1-dependent cholesterol transport. Proc Natl Acad Sci USA, 2004. 101(35): p. 13032-7.
28. Shao, B., C. Bergt, X. Fu, P. Green, J. C. Voss, M. N. Oda, J. F. Oram, and J. W. Heinecke, Tyrosine 192 in apolipoprotein A-I is the major site of nitration and chlorination by myeloperoxidase, but only chlorination markedly impairs ABCA1-dependent cholesterol transport. J Biol Chem, 2005. 280(7): p. 5983-93.
29. Zheng, L., B. Nukuna, M. L. Brennan, M. Sun, M. Goormastic, M. Settle, D. Schmitt, X. Fu, L. Thomson, P. L. Fox, H. Ischiropoulos, J. D. Smith, M. Kinter, and S. L. Hazen, Apolipoprotein A-I is a selective target for myeloperoxidase-catalyzed oxidation and functional impairment in subjects with cardiovascular disease. J Clin Invest, 2004. 114(4): p. 529-41.
30. Shao, B., S. Pennathur, I. Pagani, M. N. Oda, J. L. Witztum, J. F. Oram, and J. W. Heinecke, Modifying apolipoprotein A-I by malondialdehyde, but not by an array of other reactive carbonyls, blocks cholesterol efflux by the ABCA1 pathway. J Biol Chem, 2010. 285(24): p. 18473-84.
31. Shao, B., M. N. Oda, C. Bergt, X. Fu, P. S. Green, N. Brot, J. F. Oram, and J. W. Heinecke, Myeloperoxidase impairs ABCA1-dependent cholesterol efflux through methionine oxidation and site-specific tyrosine chlorination of apolipoprotein A-I. J Biol Chem, 2006. 281(14): p. 9001-4.
32. Shao, B., G. Cavigiolio, N. Brot, M. N. Oda, and J. W. Heinecke, Methionine oxidation impairs reverse cholesterol transport by apolipoprotein A-I. Proc Natl Acad Sci USA, 2008. 105(34): p. 12224-9.
33. de la Llera-Moya, M., D. Drazul-Schrader, B. F. Asztalos, M. Cuchel, D. J. Rader, and G. H. Rothblat, The ability to promote efflux via ABCA1 determines the capacity of serum specimens with similar high-density lipoprotein cholesterol to remove cholesterol from macrophages. Arterioscler Thromb Vasc Biol, 2010. 30(4): p. 796-801.
34. Khera, A. V., M. Cuchel, M. de la Llera-Moya, A. Rodrigues, M. F. Burke, K. Jafri, B. C. French, J. A. Phillips, M. L. Mucksavage, R. L. Wilensky, E. R. Mohler, G. H. Rothblat, and D. J. Rader, Cholesterol efflux capacity, high-density lipoprotein function, and atherosclerosis. N Engl J Med, 2011. 364(2): p. 127-35.
35. Mora, S., J. D. Otvos, N. Rifai, R. S. Rosenson, J. E. Buring, and P. M. Ridker, Lipoprotein particle profiles by nuclear magnetic resonance compared with standard lipids and apolipoproteins in predicting incident cardiovascular disease in women. Circulation, 2009. 119(7): p. 931-9.
36. Lund-Katz, S. and M. C. Phillips, High density lipoprotein structure-function and role in reverse cholesterol transport. Subcell Biochem, 2010. 51: p. 183-227.
37. Fidge, N. H., High density lipoprotein receptors, binding proteins, and ligands. J Lipid Res, 1999. 40(2): p. 187-201.
38. Cavigiolio, G., E. G. Geier, B. Shao, J. W. Heinecke, and M. N. Oda, Exchange of apolipoprotein A-I between lipid-associated and lipid-free states: a potential target for oxidative generation of dysfunctional high density lipoproteins. J Biol Chem, 2010. 285(24): p. 18847-57.
39. Lund-Katz, S., D. Nguyen, P. Dhanasekaran, M. Kono, M. Nickel, H. Saito, and M. C. Phillips, Surface plasmon resonance analysis of the mechanism of binding of apoA-I to high density lipoprotein particles. J Lipid Res, 2010. 51(3): p. 606-17.
40. Lewis, G. F. and D. J. Rader, New insights into the regulation of HDL metabolism and reverse cholesterol transport. Circ Res, 2005. 96(12): p. 1221-32.
41. Curtiss, L. K., D. T. Valenta, N. J. Hime, and K. A. Rye, What is so special about apolipoprotein AI in reverse cholesterol transport? Arterioscler Thromb Vasc Biol, 2006. 26(1): p. 12-9.
42. Rye, K. A. and P. J. Barter, Formation and metabolism of prebeta-migrating, lipid-poor apolipoprotein A-I. Arterioscler Thromb Vasc Biol, 2004. 24(3): p. 421-8.
43. Duong, P. T., G. L. Weibel, S. Lund-Katz, G. H. Rothblat, and M. C. Phillips, Characterization and properties of pre beta-HDL particles formed by ABCA1-mediated cellular lipid efflux to apoA-I. J Lipid Res, 2008. 49(5): p. 1006-14.
44. Wang, X. and D. J. Rader, Molecular regulation of macrophage reverse cholesterol transport. Curr Opin Cardiol, 2007. 22(4): p. 368-72.
45. Van Eck, M., R. R. Singaraja, D. Ye, R. B. Hildebrand, E. R. James, M. R. Hayden, and T. J. Van Berkel, Macrophage ATP-binding cassette transporter A1 overexpression inhibits atherosclerotic lesion progression in low-density lipoprotein receptor knockout mice. Arterioscler Thromb Vasc Biol, 2006. 26(4): p. 929-34.
46. Adorni, M. P., F. Zimetti, J. T. Billheimer, N. Wang, D. J. Rader, M. C. Phillips, and G. H. Rothblat, The roles of different pathways in the release of cholesterol from macrophages. J Lipid Res, 2007. 48(11): p. 2453-62.
47. Wang, N., D. L. Silver, P. Costet, and A. R. Tall, Specific binding of ApoA-I, enhanced cholesterol efflux, and altered plasma membrane morphology in cells expressing ABC1. J Biol Chem, 2000. 275(42): p. 33053-8.
48. Mulya, A., J. Y. Lee, A. K. Gebre, M. J. Thomas, P. L. Colvin, and J. S. Parks, Minimal lipidation of pre-beta HDL by ABCA1 results in reduced ability to interact with ABCA1. Arterioscler Thromb Vasc Biol, 2007. 27(8): p. 1828-36.
49. Sacks, F. M., L. L. Rudel, A. Conner, H. Akeefe, G. Kostner, T. Baki, G. Rothblat, M. de la Llera-Moya, B. Asztalos, T. Perlman, C. Zheng, P. Alaupovic, J. A. Maltais, and H. B. Brewer, Selective delipidation of plasma HDL enhances reverse cholesterol transport in vivo. J Lipid Res, 2009. 50(5): p. 894-907.
50. Gursky, O., Apolipoprotein structure and dynamics. Curr Opin Lipidol, 2005. 16(3): p. 287-94.
51. Cavigiolio, G., B. Shao, E. G. Geier, G. Ren, J. W. Heinecke, and M. N. Oda, The interplay between size, morphology, stability, and functionality of high-density lipoprotein subclasses. Biochemistry, 2008. 47(16): p. 4770-9.
52. Rye, K. A., M. A. Clay, and P. J. Barter, Remodelling of high density lipoproteins by plasma factors. Atherosclerosis, 1999. 145(2): p. 227-38.
53. Rye, K. A., N. J. Hime, and P. J. Barter, Evidence that cholesteryl ester transfer protein-mediated reductions in reconstituted high density lipoprotein size involve particle fusion. Biol Chem, 1997. 272(7): p. 3953-60.
54. Liang, H. Q., K. A. Rye, and P. J. Barter, Cycling of apolipoprotein A-I between lipid-associated and lipid-free pools. Biochim Biophys Acta, 1995. 1257(1): p. 31-7.
55. Liang, H. Q., K. A. Rye, and P. J. Barter, Remodelling of reconstituted high density lipoproteins by lecithin: cholesterol acyltransferase. J Lipid Res, 1996. 37(9): p. 1962-70.
56. Lusa, S., M. Jauhiainen, J. Metso, P. Somerharju, and C. Ehnholm, The mechanism of human plasma phospholipid transfer protein-induced enlargement of high-density lipoprotein particles: evidence for particle fusion. Biochem J, 1996. 313 (Pt 1): p. 275-82.
57. Ryan, R. O., S. Yokoyama, H. Liu, H. Czarnecka, K. Oikawa, and C. M. Kay, Human apolipoprotein A-I liberated from high-density lipoprotein without denaturation. Biochemistry, 1992. 31(18): p. 4509-14.
58. Clay, M. A., H. H. Newnham, and P. J. Barter, Hepatic lipase promotes a loss of apolipoprotein A-I from triglyceride-enriched human high density lipoproteins during incubation in vitro. Arterioscler Thromb, 1991. 11(2): p. 415-22.
59. Brouillette, C. G., W. J. Dong, Z. W. Yang, M. J. Ray, Protasevich, II, H. C. Cheung, and J. A. Engler, Forster resonance energy transfer measurements are consistent with a helical bundle model for lipid-free apolipoprotein A-I. Biochemistry, 2005. 44(50): p. 16413-25.
60. Peng, D. Q., G. Brubaker, Z. Wu, L. Zheng, B. Willard, M. Kinter, S. L. Hazen, and J. D. Smith, Apolipoprotein A-I tryptophan substitution leads to resistance to myeloperoxidase-mediated loss of function. Arterioscler Thromb Vasc Biol, 2008. 28(11): p. 2063-70.
61. Oda, M. N., T. M. Forte, R. O. Ryan, and J. C. Voss, The C-terminal domain of apolipoprotein A-I contains a lipid-sensitive conformational trigger. Nat Struct Biol, 2003. 10(6): p. 455-60.
62. Lagerstedt, J. O., G. Cavigiolio, M. S. Budamagunta, M. N. Oda, and J. C. Voss, Mapping the structural transition in an amyloidogenic apolipoprotein A-I. Biochemistry, 2007. In Press.
63. Lagerstedt, J. O., M. S. Budamagunta, M. N. Oda, and J. C. Voss, Electron paramagnetic resonance spectroscopy of site-directed spin labels reveals the structural heterogeneity in the N-terminal domain of apoA-I in solution. J Biol Chem, 2007. 282(12): p. 9143-9.
64. Martin, D. D., M. S. Budamagunta, R. O. Ryan, J. C. Voss, and M. N. Oda, Apolipoprotein A-I assumes a “looped belt” conformation on reconstituted high density lipoprotein. J Biol Chem, 2006. 281(29): p. 20418-26.
65. Lagerstedt, J. O., G. Cavigiolio, M. S. Budamagunta, I. Pagani, J. C. Voss, and M. N. Oda, Structure of apolipoprotein a-I N terminus on nascent high density lipoproteins. J Biol Chem, 2011. 286(4): p. 2966-75.
66. Gaut, J. P., J. Byun, H. D. Tran, W. M. Lauber, J. A. Carroll, R. S. Hotchkiss, A. Belaaouaj, and J. W. Heinecke, Myeloperoxidase produces nitrating oxidants in vivo. J Clin Invest, 2002. 109(10): p. 1311-9.
67. Shao, B., C. Tang, J. W. Heinecke, and J. F. Oram, Oxidation of apolipoprotein A-I by myeloperoxidase impairs the initial interactions with ABCA1 required for signaling and cholesterol export. J Lipid Res, 2010. 51(7): p. 1849-58.
68. Artola, R. L., C. B. Conde, L. Bagatolli, R. P. Pecora, G. D. Fidelio, and S. C. Kivatinitz, High-density lipoprotein from hypercholesterolemic animals has peroxidized lipids and oligomeric apolipoprotein A-I: its putative role in atherogenesis. Biochem Biophys Res Commun, 1997. 239(2): p. 570-4.
69. Malle, E., G. Marsche, U. Panzenboeck, and W. Sattler, Myeloperoxidase-mediated oxidation of high-density lipoproteins: fingerprints of newly recognized potential proatherogenic lipoproteins. Arch Biochem Biophys, 2006. 445(2): p. 245-55.
70. Bernhard, D., A. Csordas, B. Henderson, A. Rossmann, M. Kind, and G. Wick, Cigarette smoke metal-catalyzed protein oxidation leads to vascular endothelial cell contraction by depolymerization of microtubules. FASEB J, 2005. 19(9): p. 1096-107.
71. Ambrose, J. A. and R. S. Barua, The pathophysiology of cigarette smoking and cardiovascular disease: an update. J Am Coll Cardiol, 2004. 43(10): p. 1731-7.
72. Bielicki, J. K., M. R. McCall, J. J. van den Berg, F. A. Kuypers, and T. M. Forte, Copper and gas-phase cigarette smoke inhibit plasma lecithin: cholesterol acyltransferase activity by different mechanisms. J Lipid Res, 1995. 36(2): p. 322-31.
73. Columbus, L., T. Kalai, J. Jeko, K. Hideg, and W. L. Hubbell, Molecular motion of spin labeled side chains in alpha-helices: analysis by variation of side chain structure. Biochemistry, 2001. 40(13): p. 3828-46.
74. McHaourab, H. S., M. A. Lietzow, K. Hideg, and W. L. Hubbell, Motion of spin-labeled side chains in T4 lysozyme. Correlation with protein structure and dynamics. Biochemistry, 1996. 35(24): p. 7692-704.
75. Leroy, A., K. L. Toohill, J. C. Fruchart, and A. Jonas, Structural properties of high density lipoprotein subclasses homogeneous in protein composition and size. J Biol Chem, 1993. 268(7): p. 4798-805.
76. Maiorano, J. N., R. J. Jandacek, E. M. Horace, and W. S. Davidson, Identification and structural ramifications of a hinge domain in apolipoprotein A-I discoidal high-density lipoproteins of different size. Biochemistry, 2004. 43(37): p. 11717-26.
77. Noble, R. P., Electrophoretic separation of plasma lipoproteins in agarose gel. J Lipid Res, 1968. 9(6): p. 693-700.
78. Tetali, S. D., M. S. Budamagunta, J. C. Voss, and J. C. Rutledge, C-terminal interactions of apolipoprotein E4 respond to the postprandial state. J Lipid Res, 2006. 47(7): p. 1358-65.
79. Rye, K. A., K. Wee, L. K. Curtiss, D. J. Bonnet, and P. J. Barter, Apolipoprotein A-II inhibits high density lipoprotein remodeling and lipid-poor apolipoprotein A-I formation. J Biol Chem, 2003. 278(25): p. 22530-6.
80. Hennessy, L. K., S. T. Kunitake, and J. P. Kane, Apolipoprotein A-I-containing lipoproteins, with or without apolipoprotein A-II, as progenitors of pre-beta high-density lipoprotein particles. Biochemistry, 1993. 32(22): p. 5759-65.
81. Van Lenten, B. J., S. Y. Hama, F. C. de Beer, D. M. Stafforini, T. M. McIntyre, S. M. Prescott, B. N. La Du, A. M. Fogelman, and M. Navab, Anti-inflammatory HDL becomes pro-inflammatory during the acute phase response. Loss of protective effect of HDL against LDL oxidation in aortic wall cell cocultures. J Clin Invest, 1995. 96(6): p. 2758-67.
82. Vaisar, T., B. Shao, P. S. Green, M. N. Oda, J. F. Oram, and J. W. Heinecke, Myeloperoxidase and inflammatory proteins: pathways for generating dysfunctional high-density lipoprotein in humans. Curr Atheroscler Rep, 2007. 9(5): p. 417-24.
83. Heinecke, J. W., The HDL proteome: a marker—and perhaps mediator—of coronary artery disease. J Lipid Res, 2009. 50 Suppl: p. S167-71.
84. Gidez, L. I., G. J. Miller, M. Burstein, S. Slagle, and H. A. Eder, Separation and quantitation of subclasses of human plasma high density lipoproteins by a simple precipitation procedure. J Lipid Res, 1982. 23(8): p. 1206-23.
85. El Harchaoui, K., B. J. Arsenault, R. Franssen, J. P. Despres, G. K. Hovingh, E. S. Stroes, J. D. Otvos, N. J. Wareham, J. J. Kastelein, K. T. Khaw, and S. M. Boekholdt, High-density lipoprotein particle size and concentration and coronary risk. Ann Intern Med, 2009. 150(2): p. 84-93.
86. Sparks, D. L., W. S. Davidson, S. Lund-Katz, and M. C. Phillips, Effects of the neutral lipid content of high density lipoprotein on apolipoprotein A-I structure and particle stability. J Biol Chem, 1995. 270(45): p. 26910-7.
87. Sparks, D. L., P. G. Frank, and T. A. Neville, Effect of the surface lipid composition of reconstituted LPA-I on apolipoprotein A-I structure and lecithin: cholesterol acyltransferase activity. Biochim Biophys Acta, 1998. 1390(2): p. 160-72.
88. Tsuda, K., Adiponectin and membrane fluidity of erythrocytes in normotensive and hypertensive men. Obesity (Silver Spring), 2006. 14(9): p. 1505-10.
89. Tsuda, K., Y. Kinoshita, I. Nishio, and Y. Masuyama, Role of insulin in the regulation of membrane fluidity of erythrocytes in essential hypertension: an electron paramagnetic resonance investigation. Am J Hypertens, 2000. 13(4 Pt 1): p. 376-82.
90. Lindgren, F. T., H. A. Elliott, and J. W. Gofman, The ultracentrifugal characterization and isolation of human blood lipids and lipoproteins, with applications to the study of atherosclerosis. J Phys Colloid Chem, 1951. 55(1): p. 80-93.
91. Cheung, M. C. and J. J. Albers, Characterization of lipoprotein particles isolated by immunoaffinity chromatography. Particles containing A-I and A-II and particles containing A-I but no A-II. J Biol Chem, 1984. 259(19): p. 12201-9.
92. Vaisar, T., S. Pennathur, P. S. Green, S. A. Gharib, A. N. Hoofnagle, M. C. Cheung, J. Byun, S. Vuletic, S. Kassim, P. Singh, H. Chea, R. H. Knopp, J. Brunzell, R. Geary, A. Chait, X. Q. Zhao, K. Elkon, S. Marcovina, P. Ridker, J. F. Oram, and J. W. Heinecke, Shotgun proteomics implicates protease inhibition and complement activation in the antiinflammatory properties of HDL. J Clin Invest, 2007. 117(3): p. 746-56.
93. Green, P. S., T. Vaisar, S. Pennathur, J. J. Kulstad, A. B. Moore, S. Marcovina, J. Brunzell, R. H. Knopp, X. Q. Zhao, and J. W. Heinecke, Combined statin and niacin therapy remodels the high-density lipoprotein proteome. Circulation, 2008. 118(12): p. 1259-67.

1. A method for measuring capacity of high density lipoprotein (HDL) to support reverse cholesterol transport in blood, the method comprising
a) adding a spin-labeled lipoprotein probe to an in vitro blood sample, wherein the spin-labeled lipoprotein probe has high specificity for HDL,
b) collecting the electron paramagnetic resonance (EPR) spectrum of the sample.

2. The method of claim 1, further comprising the step of
c) comparing the binding of the spin-labeled lipoprotein probe to HDL by comparing the spectrum of step b) with a positive control and/or a negative control.

3-4. (canceled)

5. The method of claim 1, wherein binding efficiency of the spin-labeled probe to HDL is representative of HDL's cholesterol efflux potential.

6. The method of claim 1, wherein an amplitude of a center peak of the EPR spectrum is measured.

7-19. (canceled)

20. The method of claim 1, wherein the in vitro blood sample is a human blood sample.

21. (canceled)

22. The method of claim 1, wherein the spin-labeled lipoprotein probe comprises a first spin-label and a second spin label.

23. (canceled)

24. The method of claim 1, wherein the spin-label is covalently attached to the lipoprotein.

25. (canceled)

26. The method of claim 1, wherein the spin-labeled lipoprotein probe comprises an apoA-I or a fragment thereof, wherein the apoA-I or a fragment thereof has high specificity for HDL.

27. (canceled)

28. The method of claim 26, wherein the spin label is covalently attached to an amino acid at a single site on the apoA-I lipoprotein or fragment thereof.

29-39. (canceled)

40. The method of claim 1, wherein the spin-labeled lipoprotein probe comprises an apoE lipoprotein or fragment thereof, wherein the apoE or a fragment thereof has high specificity for HDL.

41-43. (canceled)

44. The method of claim 1, wherein the spin-labeled lipoprotein probe comprises an apoA-I mimetic, wherein the apoA-I mimetic has high specificity for HDL.

45. (canceled)

46. The method of claim 44, wherein the spin label is covalently attached to a single site on the apoA-I mimetic.

47-48. (canceled)

49. The method of claim 28, wherein the spin label is (1-oxyl-2,2,5,5-tetramethyl-Δ3-pyrroline-3-methyl)methanethiosulfonate; (1-oxyl-2,2,5,5-tetramethyl-Δ3-pyrroline-3-methyl)methanethiosulfonate-15N; 1-oxyl-2,2,5,5-tetramethyl-Δ3-pyrroline-3-methyl)Methanethiosulfonate-15N,d15; (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; (+)-(1-oxyl-2,2,5,5-tetramethylpyrrolidin-3-yl)methyl methanesulfonate; 3-(2-iodoacetamido)-PROXYL; 3-Iodomethyl-(1-oxy-2,2,5,5-tetramethylpyrroline); 1-oxyl-3-(maleimidomethyl)-2,2,5,5-tetramethyl-1-pyrrolidine; (1-oxyl-2,2,3,5,5-pentamethyl-Δ3-pyrroline-3-methyl) methanethiosulfonate; N-(1-oxyl-2,2,6,6-tetramethyl-4-piperidinyl)maleimide; (1-oxyl-2,2,5,5-tetramethylpyrrolidin-3-yl)methyl methanethiosulfonate; (1-oxyl-2,2,5,5-tetramethylpyrroline-3-yl)carbamidoethyl methanethiosulfonate; (1-oxyl-2,2,5,5-tetramethylpyrroline-3-yl)carbamidohexyl methanethiosulfonate; (1-oxyl-2,2,5,5-tetramethylpyrroline-3-yl)carbamidopropylmethane methanethiosulfonate; 3-(2-bromoacetamido)-2,2,5,5-tetramethyl-1-pyrrolidinyloxy, Free Radical; 4-bromo-3-hydroxymethyl-1-oxyl-2,2,5,5-tetramethyl-Δ3-pyrroline; 3-Bromomethyl-2,5-dihydro-2,2,5,5-tetramethyl-1H-pyrrol-1-yloxy; 4-Bromo-(1-oxyl-2,2,5,5-tetramethyl-Δ3-pyrroline-3-methyl)Methanethiosulfonate;3-[2-(2-maleimidoethoxy)ethylcarbamoyl]-PROXYL; 3-maleimido-PROXL, 3-(2-maleimidoethyl-carbamoyl)-PROXYL, free radical; 3-(3-(2-iodo-acetamido)-propyl-carbamoyl)-PROXYL, free radical; 3-(2-bromo-acetamido-methyl)-PROXYL, free radical; or 3-(2-iodo-acetamido-methyl)-PROXYL, free radical.

50. The method of claim 49, wherein the spin-label is a perdeuterated spin-label.

51. The method of claim 49, wherein the spin label is attached to an amino acid on the lipoprotein through a thiosulfonate moiety.

52. The method of claim 49, wherein the spin label further comprises a spacer moiety between the spin label and the lipoprotein.

53-57. (canceled)

58. The method of claim 1, wherein the EPR spectrum is collected at one or more timepoints after addition of the spin-labeled lipoprotein probe to the in vitro blood sample.

59-63. (canceled)

64. The method of claim 1, wherein the evaluation of step c) is a determination of the transition temperature of the HDL, wherein a transition temperature of the HDL of 25° C. or higher is indicative of a reduction in reverse cholesterol transport capacity.

65-68. (canceled)

69. The method of claim 1, wherein the in vitro blood sample further comprises an anti-coagulant.

70. (canceled)

71. A method for determining a risk for developing cardiovascular disease in a first individual; the method comprising
a) determining the reverse cholesterol transport capacity of an in vitro blood sample from the first individual according to claim 1.

72. The method of claim 71, further comprising
b) comparing the reverse cholesterol transport capacity of step a) with the reverse transport capacities of blood samples from one or more second individuals not at apparent risk of cardiovascular disease, wherein a reduction of the reverse cholesterol transport capacity of the in vitro blood sample from the first individual relative to the one or more second individuals is indicative of increased risk of cardiovascular disease.

73-75. (canceled)

76. A method for determining a risk for developing cardiovascular disease in a first individual; the method comprising
a) determining the reverse cholesterol transport capacity of an in vitro blood sample from the first individual according to claim 1.

77. The method of claim 76, further comprising
b) determining the reverse cholesterol transport capacity of an in vitro blood sample from the individual one of more times during and/or after administering the therapy to the individual, wherein an increase in the reverse transport capacity of blood samples from the individual is indicative of therapeutic efficacy.

78-79. (canceled)

80. A method for determining efficacy of a known or potential therapy for cardiovascular disease, the method comprising,
a) determining the reverse cholesterol transport capacity of an in vitro blood sample from a test individual according to claim 1, wherein the test animal has been subjected to the therapy.

81-84. (canceled)

85. A kit for measuring in in vitro blood samples capacity of high density lipoprotein (HDL) to support reverse cholesterol transport by EPR, the kit comprising a spin-labeled lipoprotein probe wherein the spin-labeled lipoprotein has high specificity for HDL.

86-133. (canceled)

134. A composition comprising an apoA-II lipoprotein or fragment thereof, wherein the apoA-II lipoprotein comprises a spin label, wherein the apoA-II lipoprotein or fragment thereof has high specificity for HDL.

135-139. (canceled)

140. A composition comprising an apoA-I lipoprotein or fragment thereof; wherein the apoA-I lipoprotein or fragment thereof comprises a spin-label and wherein the spin-label comprises (1-oxyl-2,2,5,5-tetramethylpyrrolidin-3-yl)methyl methanesulfonate; (−)-(1-oxyl-2,2,5,5-tetramethylpyrrolidin-3-yl)methyl methanesulfonate; or (+)-(1-oxyl-2,2,5,5-tetramethylpyrrolidin-3-yl)methyl methanesulfonate.

141-144. (canceled)

145. A composition comprising an in vitro blood sample and a spin-labeled lipoprotein probe wherein the spin-labeled lipoprotein has high specificity for HDL.

146-184. (canceled)

185. A composition for measuring the capacity of HDL to support reverse cholesterol transport comprising a test strip, wherein the test strip comprises a spin-labeled lipoprotein probe and a solid support, wherein the spin-labeled lipoprotein probe comprises a spin label and a protein and wherein the spin-labeled lipoprotein probe has high specificity for HDL.

186-243. (canceled)

244. A kit for measuring by EPR an in vitro sample's capacity of HDL to support reverse cholesterol transport, the kit comprising a test strip comprising a solid support, and a spin-labeled lipoprotein probe, wherein the spin-labeled lipoprotein probe comprises a spin label and a protein and wherein the spin-labeled lipoprotein probe has high specificity for HDL.

245-318. (canceled)

319. A method for measuring capacity of high density lipoprotein (HDL) to support reverse cholesterol transport in a sample, the method comprising
a) contacting an in vitro sample with a test strip comprising a solid support a spin-labeled lipoprotein probe, wherein the spin-labeled lipoprotein probe comprises a spin-label and a lipoprotein, and wherein the spin-labeled lipoprotein probe has high specificity for HDL,
b) collecting the electron paramagnetic resonance (EPR) spectrum of the spin-labeled lipoprotein probe on the test strip.

320-330. (canceled)

331. A method for screening a candidate therapeutic for modulation of cholesterol efflux capacity blood of an individual, the method comprising
a) contacting an in vitro sample with low cholesterol efflux capacity with a test strip comprising a solid support and a spin-labeled lipoprotein probe, wherein the spin-labeled lipoprotein probe comprises a spin-label and a lipoprotein and wherein the spin-labeled lipoprotein probe has high specificity for HDL,
b) contacting the sample with the candidate composition,
b) collecting the electron paramagnetic resonance (EPR) spectrum of the spin-labeled lipoprotein probe on the test strip,
wherein an increase in the cholesterol efflux potential of the sample indicates that the composition may be useful to modulate cholesterol efflux capacity.

332-383. (canceled)
